(19) United States
(12) Patent Application Publication
(10) Pub. No.: US 2026/0248815 A1
(43) Pub. Date: Aug. 27, 2026

(54) NEUROACTIVE STEROID FORMULATIONS AND METHODS OF USE THEREOF

(71) Applicant: Sage Therapeutics, LLC, Wilmington, DE (US)

(72) Inventors: Jason Legassie, Wilmington, DE (US); Padma Narayan, Wilmington, DE (US); Pam Hirtzer, Wilmington, DE (US); Christopher Galli, Wilmington, DE (US); Jeffrey Alan Wald, Wilmington, DE (US)

(21) Appl. No.: 19/489,203

(22) PCT Filed: May 2, 2024
(86) PCT No.: PCT/US2024/027544

§ 371 Date: Dec. 1, 2025

Related U.S. Application Data
(60) Provisional application No. 63/463,540, filed on May 2, 2023.

(51) Int. Cl. A61K 31/575; A61K 9/48; A61K 47/10; A61K 47/14; A61K 47/22; A61K 47/26; A61P 25/28

(52) U.S. Cl. CPC A61K 31/575; A61K 9/4858; A61K 47/10; A61K 47/14; A61K 47/22; A61K 47/26; A61P 25/28

(57) ABSTRACT

Described herein are pharmaceutical compositions comprising Compound I, and pharmaceutically acceptable salts thereof, and methods of use thereof for treating conditions associated with NMDA receptor modulation.

### CROSS REFERENCE TO RELATED APPLICATIONS

This application claims priority to and the benefit of U.S. Provisional Application No. 63/463,540, filed May 2, 2023, the disclosure of which is incorporated by reference herein in its entirety.

### BACKGROUND

NMDA receptors are highly expressed in the CNS and are involved in excitatory synaptic transmission. Activating these receptors contributes to synaptic plasticity in some circumstances and excitotoxicity in others. These receptors are ligand-gated ion channels that admit Ca²⁺ after binding of the neurotransmitters glutamate and glycine, and are fundamental to excitatory neurotransmission and normal CNS function. NMDA receptors are heteromeric complexes comprised of NR1, NR2, and/or NR3 subunits and possess distinct recognition sites for exogenous and endogenous ligands. These recognition sites include binding sites for glycine, and glutamate agonists and modulators. Positive modulators may be useful as therapeutic agents with potential clinical uses as cognitive enhancers and in the treatment of psychiatric disorders in which glutamatergic transmission is reduced or defective (see, e.g., Horak et al., J. Neuroscience, 2004, 24(46), 10318-10325). In contrast, negative modulators may be useful as therapeutic agents with potential clinical uses in the treatment of psychiatric disorders in which glutamatergic transmission is pathologically increased (e.g., treatment resistant depression).

There remains a need for pharmaceutical compositions for treating CNS diseases related to NMDA receptor modulation.

### SUMMARY OF INVENTION

Described herein are pharmaceutical compositions comprising Compound I and methods of use thereof. Also described herein is the use of pharmaceutical compositions for the manufacture of a medicament for the treatment of diseases or conditions associated with NMDA receptor modulation. Compound I is an NDMA modulator. Further described herein are pharmaceutical compositions comprising Compound I for use in treating diseases or conditions associated with NMDA receptor modulation.

In one aspect the disclosure provides a pharmaceutical composition comprising a) Compound I.

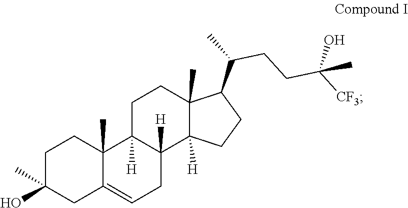

or a pharmaceutically acceptable salt thereof, and b) one or more glycerides, wherein said glyceride comprises between about 30% and about 100% w/w monoglycerides.

In some embodiments, the pharmaceutical composition comprises Compound I. In some embodiments, the pharmaceutical composition comprises a pharmaceutically acceptable salt of Compound I. In some embodiments, the pharmaceutical composition comprises about 0.2% to about 0.4% w/w Compound I. In some embodiments, the pharmaceutical composition comprises about 0.25% to about 0.35% w/w Compound I. In some embodiments, the pharmaceutical composition comprises about 0.3% w/w Compound I. In some embodiments, the pharmaceutical composition comprises 0.3% w/w Compound I. In some embodiments, the pharmaceutical composition comprises about 3 mg/g of Compound I. In some embodiments, the pharmaceutical composition comprises 3 mg/g of Compound I.

In some embodiments, the one or more glycerides is selected from the group consisting of glyceryl mono-/di-caprylocaprate, glyceryl mono-/di-caprylocaprate, glyceryl monocaprylate Type 1, glyceryl monocaprylate Type 1, glyceryl monocaprylocaprate Type 1, glyceryl monolinoleate, and combinations thereof. In some embodiments, the one or more glycerides is selected from the group consisting of glyceryl monocaprylate, glyceryl monolinoleate and a combination thereof. In some embodiments, the pharmaceutical composition comprises glyceryl monocaprylate. In some embodiments, the pharmaceutical composition comprises glyceryl monolinoleate. In some embodiments, the pharmaceutical composition comprises glyceryl monocaprylate and glyceryl monolinoleate.

In some embodiments, the amount of the one or more glycerides is from about 20% w/w to about 80% w/w. In some embodiments, wherein the amount of the one or more glycerides is from about 20% w/w to about 60% w/w.

In some embodiments, the amount of glyceryl monocaprylate in the composition is from about 50% w/w to about 80% w/w. In some embodiments, the amount of glyceryl monocaprylate is from about 55% w/w to about 60% w/w. In some embodiments, the amount of glyceryl monocaprylate is about 50% w/w. In some embodiments, the amount of glyceryl monocaprylate is 50% w/w. In some embodiments, the amount of glyceryl monocaprylate is about 60% w/w. In some embodiments, the amount of glyceryl monocaprylate is 60% w/w. In some embodiments, the amount of glyceryl monolinoleate is from about 20% w/w to about 30% w/w. In some embodiments, the amount of glyceryl monolinoleate is about 20% w/w. In some embodiments, the amount of glyceryl monolinoleate is 20% w/w. In some embodiments, the amount of glyceryl monolinoleate is about 22.5% w/w. In some embodiments, the amount of glyceryl monolinoleate is 22.5% w/w.

In some embodiments, the pharmaceutical composition further comprises stearoyl polyoxyl-32 glyceride. In some embodiments, the amount of stearoyl polyoxyl-32 glyceride is from about 5% w/w to about 20% w/w. In some embodiments, the amount of stearoyl polyoxyl-32 glyceride is from about 10% w/w to about 20% w/w. In some embodiments, the amount of stearoyl polyoxyl-32 glyceride is about 7.5% w/w. In some embodiments, the amount of stearoyl polyoxyl-32 glyceride is 7.5% w/w. In some embodiments, the amount of stearoyl polyoxyl-32 glyceride is about 10% w/w. In some embodiments, the amount of stearoyl polyoxyl-32 glyceride is 10% w/w. In some embodiments, the amount of stearoyl polyoxyl-32 glyceride is about 20% w/w. In some embodiments, the amount of stearoyl polyoxyl-32 glyceride is 20% w/w.

In some embodiments, the pharmaceutical composition further comprises a surfactant. In some embodiments, the amount of surfactant is from about 10% w/w to about 25% w/w. In some embodiments, the amount of surfactant is about 10% w/w. In some embodiments, the amount of surfactant is 10% w/w. In some embodiments, the amount of surfactant is about 20% w/w. In some embodiments, the amount of surfactant is 20% w/w. In some embodiments, the surfactant is vitamin E TPGS.

In some embodiments, the pharmaceutical composition further comprises an antioxidant. In some embodiments, the amount of antioxidant is from about 0.005% w/w to about 0.02% w/w. In some embodiments, the amount of antioxidant is about 0.005% w/w. In some embodiments, the amount of antioxidant is 0.005% w/w. In some embodiments, the amount of antioxidant is about 0.01% w/w. In some embodiments, the amount of antioxidant is 0.01% w/w. In some embodiments, the amount of antioxidant is about 0.02% w/w. In some embodiments, the amount of antioxidant is 0.02% w/w. In some embodiments, the antioxidant is butylated hydroxyanisole (BHA).

In some embodiments, the disclosure provides a pharmaceutical composition comprising: a) about 0.3% w/w Compound I, b) about 60% w/w glyceryl monocaprylate, c) about 20% w/w glyceryl monolinoleate, d) about 20% w/w vitamin E TPGS, and e) about 0.01% w/w butylated hydroxyanisole. In some embodiments, the disclosure provides a pharmaceutical composition comprising: a) about 0.3% w/w Compound I, b) about 50% w/w glyceryl monocaprylate, c) about 22.5% w/w glyceryl monolinoleate, d) about 20% w/w vitamin E TPGS, e) about 7.5% w/w stearoyl polyoxyl-32 glyceride, and f) about 0.01% w/w butylated hydroxyanisole.

In some embodiments, the pharmaceutical composition is an oral dosage form. In some embodiments, the pharmaceutical composition is a lipid formulation. In some embodiments, the oral dosage form is a soft gelatin capsule, a hardshell capsule or an oral liquid.

In one aspect, the disclosure provides a soft gelatin capsule comprising a) Compound I:

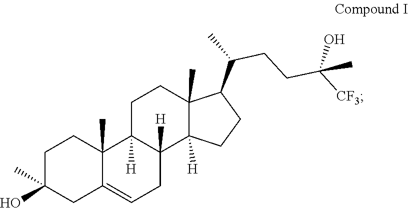

or a pharmaceutically acceptable salt thereof, and b) one or more glycerides, wherein said glycerides comprise between about 30% and about 100% w/w monoglycerides. In some embodiments, the soft gelatin capsule comprises Compound I. In some embodiments, the soft gelatin capsule comprises a pharmaceutically acceptable salt of Compound I. In some embodiments, the soft gelatin capsule comprises about 0.2% to about 0.4% w/w Compound I. In some embodiments, the soft gelatin capsule comprises about 0.25% to about 0.35% w/w Compound I. In some embodiments, the soft gelatin capsule comprises about 0.3% w/w Compound I. In some embodiments, the soft gelatin capsule comprises 0.3% w/w Compound I. In some embodiments, the soft gelatin capsule comprises about 3 mg/g of Compound I. In some embodiments, the soft gelatin capsule comprises about 3 mg/g of Compound I. In some embodiments, the one or more glycerides in the soft gelatin capsule is selected from the group consisting of glyceryl mono-/di-caprylocaprate, glyceryl mono-/di-caprylocaprate, glyceryl monocaprylate Type 1, glyceryl monocaprylate Type 1, glyceryl monocaprylocaprate Type 1, glyceryl monolinoleate, and combinations thereof. In some embodiments, the one or more glycerides in the soft gelatin capsule is selected from the group consisting of glyceryl monocaprylate, glyceryl monolinoleate and a combination thereof. In some embodiments, the soft gelatin capsule comprises glyceryl monocaprylate. In some embodiments, the soft gelatin capsule comprises glyceryl monolinoleate. In some embodiments, the soft gelatin capsule comprises glyceryl monocaprylate and glyceryl monolinoleate. In some embodiments, the amount of the one or more glycerides in the soft gelatin capsule is from about 20% w/w to about 80% w/w. In some embodiments, the amount of the one or more glycerides in the soft gelatin capsule is from about 20% w/w to about 60% w/w. In some embodiments, the amount of glyceryl monocaprylate in the soft gelatin capsule is from about 50% w/w to about 80% w/w. In some embodiments, the amount of glyceryl monocaprylate in the soft gelatin capsule is from about 55% w/w to about 60% w/w. In some embodiments, the amount of glyceryl monocaprylate in the soft gelatin capsule is about 50% w/w. In some embodiments, the amount of glyceryl monocaprylate in the soft gelatin capsule is 50% w/w. In some embodiments, the amount of glyceryl monocaprylate in the soft gelatin capsule is about 60% w/w. In some embodiments, the amount of glyceryl monocaprylate in the soft gelatin capsule is 60% w/w. In some embodiments, the amount of glyceryl monolinoleate in the soft gelatin capsule is from about 20% w/w to about 30% w/w. In some embodiments, the amount of glyceryl monolinoleate in the soft gelatin capsule is about 20% w/w. In some embodiments, the amount of glyceryl monolinoleate in the soft gelatin capsule is 20% w/w. In some embodiments, the amount of glyceryl monolinoleate in the soft gelatin capsule is about 22.5% w/w. In some embodiments, the amount of glyceryl monolinoleate in the soft gelatin capsule is 22.5% w/w.

In some embodiments, the soft gelatin capsule further comprises stearoyl polyoxyl-32 glyceride. In some embodiments, the amount of stearoyl polyoxyl-32 glyceride in the soft gelatin capsule is from about 5% w/w to about 20% w/w. In some embodiments, the amount of stearoyl polyoxyl-32 glyceride in the soft gelatin capsule is from about 10% w/w/to about 20% w/w. In some embodiments, the amount of stearoyl polyoxyl-32 glyceride in the soft gelatin capsule is about 7.5% w/w. In some embodiments, the amount of stearoyl polyoxyl-32 glyceride in the soft gelatin capsule is 7.5% w/w. In some embodiments, the amount of stearoyl polyoxyl-32 glyceride in the soft gelatin capsule is about 10% w/w. In some embodiments, the amount of stearoyl polyoxyl-32 glyceride in the soft gelatin capsule is 10% w/w. In some embodiments, the amount of stearoyl polyoxyl-32 glyceride in the soft gelatin capsule is about 20% w/w. In some embodiments, the amount of stearoyl polyoxyl-32 glyceride in the soft gelatin capsule is 20% w/w.

In some embodiments, the soft gelatin capsule further comprises a surfactant. In some embodiments, the amount of surfactant in the soft gelatin capsule is from about 10% w/w to about 25% w/w. In some embodiments, the amount of surfactant in the soft gelatin capsule is about 10% w/w. In some embodiments, the amount of surfactant in the soft gelatin capsule is 10% w/w. In some embodiments, the amount of surfactant in the soft gelatin capsule is about 20% w/w. In some embodiments, the amount of surfactant in the soft gelatin capsule is 20% w/w. In some embodiments, the surfactant is vitamin E TPGS. In some embodiments, the soft gelatin capsule further comprises an antioxidant. In some embodiments, the amount of antioxidant the soft gelatin capsule is from about 0.005% w/w to about 0.02% w/w. In some embodiments, the amount of antioxidant the soft gelatin capsule is about 0.005% w/w. In some embodiments, the amount of antioxidant the soft gelatin capsule is 0.005% w/w. In some embodiments, the amount of antioxidant the soft gelatin capsule is about 0.01% w/w. In some embodiments, the amount of antioxidant the soft gelatin capsule is 0.01% w/w. In some embodiments, the amount of antioxidant the soft gelatin capsule is about 0.02% w/w. In some embodiments, the amount of antioxidant the soft gelatin capsule is 0.02% w/w. In some embodiments, the antioxidant is butylated hydroxyanisole (BHA).

In some embodiments, the disclosure provides a soft gelatin capsule comprising: a) about 0.3% w/w Compound I, b) about 60% w/w glyceryl monocaprylate, c) about 20% w/w glyceryl monolinoleate, d) about 20% w/w vitamin E TPGS, and e) about 0.01% w/w butylated hydroxyanisole. In some embodiments, the disclosure provides a soft gelatin capsule comprising: a) about 0.3% w/w Compound I, b) about 50% w/w glyceryl monocaprylate, c) about 22.5% w/w glyceryl monolinoleate, d) about 20% w/w vitamin E TPGS, e) about 7.5% w/w stearoyl polyoxyl-32 glyceride, and f) about 0.01% w/w butylated hydroxyanisole.

In one aspect, the disclosure provides a pharmaceutical composition comprising a therapeutically effective amount of Compound I

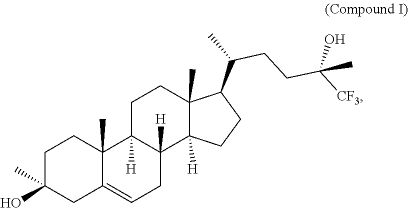

or a pharmaceutically acceptable salt thereof, and a pharmaceutically acceptable carrier;
wherein following administration of the pharmaceutical composition to a subject, the pharmaceutical composition provides one or more of the following pharmacokinetic parameters:
a) a Cₘₐₓ of between about 8 and about 25 ng/mL;
b) an AUCₗₐₛₜ of between about 100 and about 450 ng*h/mL;
c) an AUC_{inf} of between about 100 and about 500 ng*h/mL;
d) a tₘₐₓ of between about 2 and about 8 hours; or
e) a bioavailability of between about 70 and about 100%.

In one aspect, the disclosure provides a method of treating Huntington's disease in a subject, comprising administering to the subject a therapeutically effective amount of a pharmaceutical composition as disclosed herein.

In one aspect, the disclosure provides a method of treating cognitive impairment in a subject having Huntington's disease, comprising administering to the subject a therapeutically effective amount of a pharmaceutical composition as disclosed herein.

In one aspect, the disclosure provides a method of treating Mild Cognitive Impairment (MCI) associated with Huntington's disease in a subject, comprising administering to the subject a therapeutically effective amount of a pharmaceutical composition as disclosed herein.

In one aspect, the disclosure provides a method of improving executive function in a subject having Mild Cognitive Impairment (MCI) associated with Huntington's disease, comprising administering to the subject a therapeutically effective amount of a pharmaceutical composition as disclosed herein.

In one aspect, the disclosure provides a method of improving working memory in a subject having Mild Cognitive Impairment (MCI) associated with Huntington's disease, comprising administering to the subject a therapeutically effective amount of a pharmaceutical composition as disclosed herein.

In one aspect, the disclosure provides a method of improving learning in a subject having Mild Cognitive Impairment (MCI) associated with Huntington's disease, comprising administering to the subject a therapeutically effective amount of a pharmaceutical composition as disclosed herein.

In one aspect, the disclosure provides a method of improving learning and working memory in a subject having Mild Cognitive Impairment (MCI) associated with Huntington's disease, comprising administering to the subject a therapeutically effective amount of a pharmaceutical composition as disclosed herein.

In one aspect, the disclosure provides a method of improving cognition in a subject having Huntington's disease, comprising administering to the subject a therapeutically effective amount of a pharmaceutical composition as disclosed herein.

In one aspect, the disclosure provides a method for slowing cognitive decline in a subject having Huntington's disease, comprising administering to the subject a therapeutically effective amount of a pharmaceutical composition as disclosed herein.

In one aspect, the disclosure provides a method of treating Parkinson's disease in a subject, comprising administering to the subject a therapeutically effective amount of a pharmaceutical composition as disclosed herein.

In one aspect, the disclosure provides a method of treating cognitive impairment in a subject having Parkinson's disease, comprising administering to the subject a therapeutically effective amount of a pharmaceutical composition as disclosed herein.

In one aspect, the disclosure provides a method of treating Mild Cognitive Impairment (MCI) associated with Parkinson's disease in a subject, comprising administering to the subject a therapeutically effective amount of a pharmaceutical composition as disclosed herein.

In one aspect, the disclosure provides a method of improving executive function in a subject having Mild Cognitive Impairment (MCI) associated with Parkinson's disease, comprising administering to the subject a therapeutically effective amount of a pharmaceutical composition as disclosed herein.

In one aspect, the disclosure provides a method of improving working memory in a subject having Mild Cognitive Impairment (MCI) associated with Parkinson's disease, comprising administering to the subject a therapeutically effective amount of a pharmaceutical composition as disclosed herein.

In one aspect, the disclosure provides a method of improving learning in a subject having Mild Cognitive Impairment (MCI) associated with Parkinson's disease, comprising administering to the subject a therapeutically effective amount of a pharmaceutical composition as disclosed herein.

In one aspect, the disclosure provides a method of improving learning and working memory in a subject having Mild Cognitive Impairment (MCI) associated with Parkinson's disease, comprising administering to the subject a therapeutically effective amount of a pharmaceutical composition as disclosed herein.

In one aspect, the disclosure provides a method of improving cognition in a subject having Parkinson's disease, comprising administering to the subject a therapeutically effective amount of a pharmaceutical composition as disclosed herein.

In one aspect, the disclosure provides a method for slowing cognitive decline in a subject having Parkinson's disease, comprising administering to the subject a therapeutically effective amount of a pharmaceutical composition as disclosed herein.

In one aspect, the disclosure provides a method of treating Alzheimer's disease in a subject, comprising administering to the subject a therapeutically effective amount of a pharmaceutical composition as disclosed herein.

In one aspect, the disclosure provides a method of treating cognitive impairment in a subject having Alzheimer's disease, comprising administering to the subject a therapeutically effective amount of a pharmaceutical composition as disclosed herein.

In one aspect, the disclosure provides a method of treating Mild Cognitive Impairment (MCI) associated with Alzheimer's disease in a subject, comprising administering to the subject a therapeutically effective amount of a pharmaceutical composition as disclosed herein.

In one aspect, the disclosure provides a method of improving executive function in a subject having Mild Cognitive Impairment (MCI) associated with Alzheimer's disease, comprising administering to the subject a therapeutically effective amount of a pharmaceutical composition as disclosed herein.

In one aspect, the disclosure provides a method of improving working memory in a subject having Mild Cognitive Impairment (MCI) associated with Alzheimer's disease, comprising administering to the subject a therapeutically effective amount of a pharmaceutical composition as disclosed herein.

In one aspect, the disclosure provides a method of improving learning in a subject having Mild Cognitive Impairment (MCI) associated with Alzheimer's disease, comprising administering to the subject a therapeutically effective amount of a pharmaceutical composition as disclosed herein.

In one aspect, the disclosure provides a method of improving learning and working memory in a subject having Mild Cognitive Impairment (MCI) associated with Alzheimer's disease, comprising administering to the subject a therapeutically effective amount of a pharmaceutical composition as disclosed herein.

In one aspect, the disclosure provides a method of improving cognition in a subject having Alzheimer's disease, comprising administering to the subject a therapeutically effective amount of a pharmaceutical composition as disclosed herein.

In one aspect, the disclosure provides a method for slowing cognitive decline in a subject having Alzheimer's disease, comprising administering to the subject a therapeutically effective amount of a pharmaceutical composition as disclosed herein.

In one aspect, the disclosure provides a method of treating mild dementia associated with Alzheimer's disease, comprising administering to the subject a therapeutically effective amount of a pharmaceutical composition as disclosed herein.

In one aspect, the disclosure provides a method of improving executive function in a subject having mild dementia associated with Alzheimer's disease, comprising administering to the subject a therapeutically effective amount of a pharmaceutical composition as disclosed herein.

In one aspect, the disclosure provides a method of improving working memory in a subject having mild dementia associated with Alzheimer's disease, comprising administering to the subject a therapeutically effective amount of a pharmaceutical composition capsule as disclosed herein.

In one aspect, the disclosure provides a method of improving learning in a subject having mild dementia associated with Alzheimer's disease, comprising administering to the subject a therapeutically effective amount of a pharmaceutical composition as disclosed herein.

In one aspect, the disclosure provides a method of improving learning and working memory in a subject having mild dementia associated with Alzheimer's disease, comprising administering to the subject a therapeutically effective amount of a pharmaceutical composition as disclosed herein.

In one aspect, the disclosure provides a method of treating Huntington's disease in a subject, comprising administering to the subject a therapeutically effective amount of a soft gelatin capsule as disclosed herein.

In one aspect, the disclosure provides a method of treating cognitive impairment in a subject having Huntington's disease, comprising administering to the subject a therapeutically effective amount of a soft gelatin capsule as disclosed herein.

In one aspect, the disclosure provides a method of treating Mild Cognitive Impairment (MCI) associated with Huntington's disease in a subject, comprising administering to the subject a therapeutically effective amount of a soft gelatin capsule as disclosed herein.

In one aspect, the disclosure provides a method of improving executive function in a subject having Mild Cognitive Impairment (MCI) associated with Huntington's disease, comprising administering to the subject a therapeutically effective amount of a soft gelatin capsule as disclosed herein.

In one aspect, the disclosure provides a method of improving working memory in a subject having Mild Cognitive Impairment (MCI) associated with Huntington's disease, comprising administering to the subject a therapeutically effective amount of a soft gelatin capsule as disclosed herein.

In one aspect, the disclosure provides a method of improving learning in a subject having Mild Cognitive Impairment (MCI) associated with Huntington's disease, comprising administering to the subject a therapeutically effective amount of a soft gelatin capsule as disclosed herein.

In one aspect, the disclosure provides a method of improving learning and working memory in a subject having Mild Cognitive Impairment (MCI) associated with Huntington's disease, comprising administering to the subject a therapeutically effective amount of a soft gelatin capsule as disclosed herein.

In one aspect, the disclosure provides a method of improving cognition in a subject having Huntington's disease, comprising administering to the subject a therapeutically effective amount of a soft gelatin capsule as disclosed herein.

In one aspect, the disclosure provides a method for slowing cognitive decline in a subject having Huntington's disease, comprising administering to the subject a therapeutically effective amount of a soft gelatin capsule as disclosed herein.

In one aspect, the disclosure provides a method of treating Parkinson's disease in a subject, comprising administering to the subject a therapeutically effective amount of a soft gelatin capsule as disclosed herein.

In one aspect, the disclosure provides a method of treating cognitive impairment in a subject having Parkinson's disease, comprising administering to the subject a therapeutically effective amount of a soft gelatin capsule as disclosed herein.

In one aspect, the disclosure provides a method of treating Mild Cognitive Impairment (MCI) associated with Parkinson's disease in a subject, comprising administering to the subject a therapeutically effective amount of a soft gelatin capsule as disclosed herein.

In one aspect, the disclosure provides a method of improving executive function in a subject having Mild Cognitive Impairment (MCI) associated with Parkinson's disease, comprising administering to the subject a therapeutically effective amount of a soft gelatin capsule as disclosed herein.

In one aspect, the disclosure provides a method of improving working memory in a subject having Mild Cognitive Impairment (MCI) associated with Parkinson's disease, comprising administering to the subject a therapeutically effective amount of a soft gelatin capsule as disclosed herein.

In one aspect, the disclosure provides a method of improving learning in a subject having Mild Cognitive Impairment (MCI) associated with Parkinson's disease, comprising administering to the subject a therapeutically effective amount of a soft gelatin capsule as disclosed herein.

In one aspect, the disclosure provides a method of improving learning and working memory in a subject having Mild Cognitive Impairment (MCI) associated with Parkinson's disease, comprising administering to the subject a therapeutically effective amount of a soft gelatin capsule as disclosed herein.

In one aspect, the disclosure provides a method of improving cognition in a subject having Parkinson's disease, comprising administering to the subject a therapeutically effective amount of a soft gelatin capsule as disclosed herein.

In one aspect, the disclosure provides a method for slowing cognitive decline in a subject having Parkinson's disease, comprising administering to the subject a therapeutically effective amount of a soft gelatin capsule as disclosed herein.

In one aspect, the disclosure provides a method of treating Alzheimer's disease in a subject, comprising administering to the subject a therapeutically effective amount of a soft gelatin capsule as disclosed herein.

In one aspect, the disclosure provides a method of treating cognitive impairment in a subject having Alzheimer's disease, comprising administering to the subject a therapeutically effective amount of a soft gelatin capsule as disclosed herein.

In one aspect, the disclosure provides a method of treating Mild Cognitive Impairment (MCI) associated with Alzheimer's disease in a subject, comprising administering to the subject a therapeutically effective amount of a soft gelatin capsule as disclosed herein.

In one aspect, the disclosure provides a method of improving executive function in a subject having Mild Cognitive Impairment (MCI) associated with Alzheimer's disease, comprising administering to the subject a therapeutically effective amount of a soft gelatin capsule as disclosed herein.

In one aspect, the disclosure provides a method of improving working memory in a subject having Mild Cognitive Impairment (MCI) associated with Alzheimer's disease, comprising administering to the subject a therapeutically effective amount of a soft gelatin capsule as disclosed herein.

In one aspect, the disclosure provides a method of improving learning in a subject having Mild Cognitive Impairment (MCI) associated with Alzheimer's disease, comprising administering to the subject a therapeutically effective amount of a soft gelatin capsule as disclosed herein.

In one aspect, the disclosure provides a method of improving learning and working memory in a subject having Mild Cognitive Impairment (MCI) associated with Alzheimer's disease, comprising administering to the subject a therapeutically effective amount of a soft gelatin capsule as disclosed herein.

In one aspect, the disclosure provides a method of improving cognition in a subject having Alzheimer's disease, comprising administering to the subject a therapeutically effective amount of a soft gelatin capsule as disclosed herein.

In one aspect, the disclosure provides a method for slowing cognitive decline in a subject having Alzheimer's disease, comprising administering to the subject a therapeutically effective amount of a soft gelatin capsule as disclosed herein.

In one aspect, the disclosure provides a method of treating mild dementia associated with Alzheimer's disease, comprising administering to the subject a therapeutically effective amount of a soft gelatin capsule as disclosed herein.

In one aspect, the disclosure provides a method of improving executive function in a subject having mild dementia associated with Alzheimer's disease, comprising administering to the subject a therapeutically effective amount of a soft gelatin capsule as disclosed herein.

In one aspect, the disclosure provides a method of improving working memory in a subject having mild dementia associated with Alzheimer's disease, comprising administering to the subject a therapeutically effective amount of a soft gelatin capsule as disclosed herein.

In one aspect, the disclosure provides a method of improving learning in a subject having mild dementia associated with Alzheimer's disease, comprising administering to the subject a therapeutically effective amount of a soft gelatin capsule as disclosed herein.

In one aspect, the disclosure provides a method of improving learning and working memory in a subject having mild dementia associated with Alzheimer's disease, comprising administering to the subject a therapeutically effective amount of a soft gelatin capsule as disclosed herein.

In some embodiments, the method comprises administering to the subject a total daily dose of from about 0.3 mg to about 1.5 mg of Compound I. In some embodiments, the method comprises administering to the subject a total daily dose of from about 0.6 mg to about 1.5 mg of Compound I. In some embodiments, the method comprises administering to the subject a total daily dose of from about 0.9 mg to about 1.2 mg of Compound I. In some embodiments, the method comprises administering to the subject a total daily dose of about 1.2 mg of Compound I. In some embodiments, the method comprises administering to the subject a total daily dose of 1.2 mg of Compound I. In some embodiments, the method comprises administering to the subject a total daily dose of about 0.9 mg of Compound I. In some embodiments, the method comprises administering to the subject a total daily dose of 0.9 mg of Compound I. In some embodiments, the method comprises administering to the subject a total daily dose of about 0.6 mg of Compound I. In some embodiments, the method comprises administering to the subject a total daily dose of 0.6 mg of Compound I. In some embodiments, the method comprises administering to the subject a total daily dose of about 0.3 mg of Compound I. In some embodiments, the method comprises administering to the subject a total daily dose of 0.3 mg of Compound I.

In some embodiments, the dose of Compound I is administered to the subject at a frequency of once daily, twice daily, three times daily or four times daily.

In some embodiments, the method comprises administering to the subject at least one loading dose; and at least one maintenance dose. In some embodiments, the method comprises administering to the subject the loading dose daily. In some embodiments, the method comprises administering to the subject the loading dose daily for at least 2, 4, 6, 8 or 10 weeks. In some embodiments, the method comprises administering to the subject the loading dose daily for at least 2 weeks. In some embodiments, the method comprises administering to the subject the loading dose daily for at least 4 weeks. In some embodiments, the method comprises administering to the subject the loading dose daily for at least 6 weeks. In some embodiments, the method comprises administering to the subject the loading dose daily for at least 8 weeks. In some embodiments, the method comprises administering to the subject the loading dose daily for at least 10 weeks. In some embodiments, the loading dose is about 1.2 mg daily of Compound I. In some embodiments, the loading dose is 1.2 mg daily of Compound I. In some embodiments, the maintenance dose is administered daily. In some embodiments, the maintenance dose is about 0.9 mg daily of Compound I. In some embodiments, the maintenance dose is 0.9 mg daily of Compound I. In some embodiments, the maintenance dose is about 0.6 mg daily of Compound I. In some embodiments, the maintenance dose is 0.6 mg daily of Compound I.

In one aspect, the disclosure provides a pharmaceutical composition as disclosed herein, for use in treating Huntington's disease in a subject.

In one aspect, the disclosure provides a pharmaceutical composition as disclosed herein, for use in treating cognitive impairment in a subject having Huntington's disease.

In one aspect, the disclosure provides a pharmaceutical composition as disclosed herein, for use in treating Mild Cognitive Impairment (MCI) associated with Huntington's disease in a subject.

In one aspect, the disclosure provides a pharmaceutical composition as disclosed herein, for use in improving executive function in a subject having Mild Cognitive Impairment (MCI) associated with Huntington's disease.

In one aspect, the disclosure provides a pharmaceutical composition as disclosed herein, for use in improving working memory in a subject having Mild Cognitive Impairment (MCI) associated with Huntington's disease.

In one aspect, the disclosure provides a pharmaceutical composition as disclosed herein, for use in improving learning in a subject having Mild Cognitive Impairment (MCI) associated with Huntington's disease.

In one aspect, the disclosure provides a pharmaceutical composition as disclosed herein, for use in improving learning and working memory in a subject having Mild Cognitive Impairment (MCI) associated with Huntington's disease.

In one aspect, the disclosure provides a pharmaceutical composition as disclosed herein, for use in improving cognition in a subject having Huntington's disease.

In one aspect, the disclosure provides a pharmaceutical composition as disclosed herein, for use in slowing cognitive decline in a subject having Huntington's disease.

In one aspect, the disclosure provides a pharmaceutical composition as disclosed herein, for use in treating Parkinson's disease in a subject.

In one aspect, the disclosure provides a pharmaceutical composition as disclosed herein, for use in treating cognitive impairment in a subject having Parkinson's disease.

In one aspect, the disclosure provides a pharmaceutical composition as disclosed herein, for use in treating Mild Cognitive Impairment (MCI) associated with Parkinson's disease in a subject.

In one aspect, the disclosure provides a pharmaceutical composition as disclosed herein, for use in improving executive function in a subject having Mild Cognitive Impairment (MCI) associated with Parkinson's disease.

In one aspect, the disclosure provides a pharmaceutical composition as disclosed herein, for use in improving working memory in a subject having Mild Cognitive Impairment (MCI) associated with Parkinson's disease.

In one aspect, the disclosure provides a pharmaceutical composition as disclosed herein, for use in improving learning in a subject having Mild Cognitive Impairment (MCI) associated with Parkinson's disease.

In one aspect, the disclosure provides a pharmaceutical composition as disclosed herein, for use in improving learning and working memory in a subject having Mild Cognitive Impairment (MCI) associated with Parkinson's disease.

In one aspect, the disclosure provides a pharmaceutical composition as disclosed herein, for use in improving cognition in a subject having Parkinson's disease.

In one aspect, the disclosure provides a pharmaceutical composition as disclosed herein, for use in slowing cognitive decline in a subject having Parkinson's disease.

In one aspect, the disclosure provides a pharmaceutical composition as disclosed herein, for use in treating Alzheimer's disease in a subject.

In one aspect, the disclosure provides a pharmaceutical composition as disclosed herein, for use in treating cognitive impairment in a subject having Alzheimer's disease.

In one aspect, the disclosure provides a pharmaceutical composition as disclosed herein, for use in treating Mild Cognitive Impairment (MCI) associated with Alzheimer's disease in a subject.

In one aspect, the disclosure provides a pharmaceutical composition as disclosed herein, for use in improving executive function in a subject having Mild Cognitive Impairment (MCI) associated with Alzheimer's disease.

In one aspect, the disclosure provides a pharmaceutical composition as disclosed herein, for use in improving working memory in a subject having Mild Cognitive Impairment (MCI) associated with Alzheimer's disease.

In one aspect, the disclosure provides a pharmaceutical composition as disclosed herein, for use in improving learning in a subject having Mild Cognitive Impairment (MCI) associated with Alzheimer's disease.

In one aspect, the disclosure provides a pharmaceutical composition as disclosed herein, for use in improving learning and working memory in a subject having Mild Cognitive Impairment (MCI) associated with Alzheimer's disease.

In one aspect, the disclosure provides a pharmaceutical composition as disclosed herein, for use in improving cognition in a subject having Alzheimer's disease.

In one aspect, the disclosure provides a pharmaceutical composition as disclosed herein, for use in slowing cognitive decline in a subject having Alzheimer's disease.

In one aspect, the disclosure provides a pharmaceutical composition as disclosed herein, for use in treating mild dementia associated with Alzheimer's Disease.

In one aspect, the disclosure provides a pharmaceutical composition as disclosed herein, for use in improving executive function in a subject having mild dementia associated with Alzheimer's disease.

In one aspect, the disclosure provides a pharmaceutical composition as disclosed herein, for use in improving working memory in a subject having mild dementia associated with Alzheimer's disease.

In one aspect, the disclosure provides a pharmaceutical composition as disclosed herein, for use in improving learning in a subject having mild dementia associated with Alzheimer's disease.

In one aspect, the disclosure provides a pharmaceutical composition as disclosed herein, for use in improving learning and working memory in a subject having mild dementia associated with Alzheimer's disease.

In some embodiments, the composition is formulated for administering a total daily dose of from about 0.3 mg to about 1.5 mg of Compound I. In some embodiments, the composition is formulated for administering a total daily dose of about 1.2 mg of Compound I. In some embodiments, the composition is formulated for administering a total daily dose of 1.2 mg of Compound I. In some embodiments, the composition is formulated for administering a total daily dose of about 0.9 mg of Compound I. In some embodiments, the composition is formulated for administering a total daily dose of 0.9 mg of Compound I. In some embodiments, the composition is formulated for administering a total daily dose of about 0.6 mg of Compound I. In some embodiments, the composition is formulated for administering a total daily dose of 0.6 mg of Compound I. In some embodiments, the composition is formulated for administering a total daily dose of about 0.3 mg of Compound I. In some embodiments, the composition is formulated for administering a total daily dose of 0.3 mg of Compound I. In some embodiments, the treatment comprises administering at least one loading dose and at least one maintenance dose.

In some embodiments, the treatment comprises administering the loading dose daily for at least 2, 4, 6, 8 or 10 weeks.

In some embodiments, the treatment comprises administering the loading dose daily for at least 2 weeks. In some embodiments, the treatment comprises administering the loading dose daily for at least 4 weeks. In some embodiments, the treatment comprises administering the loading dose daily for at least 6 weeks. In some embodiments, the treatment comprises administering the loading dose daily for at least 8 weeks. In some embodiments, the treatment comprises administering the loading dose daily for at least 10 weeks. In some embodiments, the loading dose is about 1.2 mg daily of Compound I. In some embodiments, the loading dose is 1.2 mg daily of Compound I. In some embodiments, the maintenance dose is about 0.9 mg daily of Compound I. In some embodiments, the maintenance dose is administered daily. In some embodiments, the maintenance dose is 0.9 mg daily of Compound I. In some embodiments, the maintenance dose is about 0.6 mg daily of Compound I. In some embodiments, the maintenance dose is 0.6 mg daily of Compound I.

In one aspect, the disclosure provides a use of a pharmaceutical composition as disclosed herein for the manufacture of a medicament for treating Huntington's disease in a subject.

In one aspect, the disclosure provides a use of a pharmaceutical composition as disclosed herein for the manufacture of a medicament for treating cognitive impairment in a subject having Huntington's disease.

In one aspect, the disclosure provides a use of a pharmaceutical composition as disclosed herein for the manufacture of a medicament for treating Mild Cognitive Impairment (MCI) associated with Huntington's disease in a subject.

In one aspect, the disclosure provides a use of a pharmaceutical composition as disclosed herein for the manufacture of a medicament for improving executive function in a subject having Mild Cognitive Impairment (MCI) associated with Huntington's disease.

In one aspect, the disclosure provides a use of a pharmaceutical composition as disclosed herein for the manufacture of a medicament for improving working memory in a subject having Mild Cognitive Impairment (MCI) associated with Huntington's disease.

In one aspect, the disclosure provides a use of a pharmaceutical composition as disclosed herein for the manufacture of a medicament for improving learning in a subject having Mild Cognitive Impairment (MCI) associated with Huntington's disease.

In one aspect, the disclosure provides a use of a pharmaceutical composition as disclosed herein for the manufacture of a medicament for improving learning and working memory in a subject having Mild Cognitive Impairment (MCI) associated with Huntington's disease.

In one aspect, the disclosure provides a use of a pharmaceutical composition as disclosed herein for the manufacture of a medicament for improving cognition in a subject having Huntington's disease.

In one aspect, the disclosure provides a use of a pharmaceutical composition as disclosed herein for the manufacture of a medicament for slowing cognitive decline in a subject having Huntington's disease.

In one aspect, the disclosure provides a use of a pharmaceutical composition as disclosed herein for the manufacture of a medicament for treating Parkinson's disease in a subject.

In one aspect, the disclosure provides a use of a pharmaceutical composition as disclosed herein for the manufacture of a medicament for treating cognitive impairment in a subject having Parkinson's disease.

In one aspect, the disclosure provides a use of a pharmaceutical composition as disclosed herein for the manufacture of a medicament for treating Mild Cognitive Impairment (MCI) associated with Parkinson's disease in a subject.

In one aspect, the disclosure provides a use of a pharmaceutical composition as disclosed herein for the manufacture of a medicament for improving executive function in a subject having Mild Cognitive Impairment (MCI) associated with Parkinson's disease.

In one aspect, the disclosure provides a use of a pharmaceutical composition as disclosed herein for the manufacture of a medicament for improving working memory in a subject having Mild Cognitive Impairment (MCI) associated with Parkinson's disease.

In one aspect, the disclosure provides a use of a pharmaceutical composition as disclosed herein for the manufacture of a medicament for improving learning in a subject having Mild Cognitive Impairment (MCI) associated with Parkinson's disease.

In one aspect, the disclosure provides a use of a pharmaceutical composition as disclosed herein for the manufacture of a medicament for improving learning and working memory in a subject having Mild Cognitive Impairment (MCI) associated with Parkinson's disease.

In one aspect, the disclosure provides a use of a pharmaceutical composition as disclosed herein for the manufacture of a medicament for improving cognition in a subject having Parkinson's disease.

In one aspect, the disclosure provides a use of a pharmaceutical composition as disclosed herein for the manufacture of a medicament for slowing cognitive decline in a subject having Parkinson's disease.

In one aspect, the disclosure provides a use of a pharmaceutical composition as disclosed herein for the manufacture of a medicament for treating Alzheimer's disease in a subject.

In one aspect, the disclosure provides a use of a pharmaceutical composition as disclosed herein for the manufacture of a medicament for treating cognitive impairment in a subject having Alzheimer's disease.

In one aspect, the disclosure provides a use of a pharmaceutical composition as disclosed herein for the manufacture of a medicament for treating Mild Cognitive Impairment (MCI) associated with Alzheimer's disease in a subject.

In one aspect, the disclosure provides a use of a pharmaceutical composition as disclosed herein for the manufacture of a medicament for improving executive function in a subject having Mild Cognitive Impairment (MCI) associated with Alzheimer's disease.

In one aspect, the disclosure provides a use of a pharmaceutical composition as disclosed herein for the manufacture of a medicament for improving working memory in a subject having Mild Cognitive Impairment (MCI) associated with Alzheimer's disease.

In one aspect, the disclosure provides a use of a pharmaceutical composition as disclosed herein for the manufacture of a medicament for improving learning in a subject having Mild Cognitive Impairment (MCI) associated with Alzheimer's disease.

In one aspect, the disclosure provides a use of a pharmaceutical composition as disclosed herein for the manufacture of a medicament for improving learning and working memory in a subject having Mild Cognitive Impairment (MCI) associated with Alzheimer's disease.

In one aspect, the disclosure provides a use of a pharmaceutical composition as disclosed herein for the manufacture of a medicament for improving cognition in a subject having Alzheimer's disease.

In one aspect, the disclosure provides a use of a pharmaceutical composition as disclosed herein for the manufacture of a medicament for slowing cognitive decline in a subject having Alzheimer's disease.

In one aspect, the disclosure provides a use of a pharmaceutical composition as disclosed herein for the manufacture of a medicament for treating mild dementia associated with Alzheimer's Disease.

In one aspect, the disclosure provides a use of a pharmaceutical composition as disclosed herein for the manufacture of a medicament for improving executive function in a subject having mild dementia associated with Alzheimer's disease.

In one aspect, the disclosure provides a use of a pharmaceutical composition as disclosed herein for the manufacture of a medicament for improving working memory in a subject having mild dementia associated with Alzheimer's disease.

In one aspect, the disclosure provides a use of a pharmaceutical composition as disclosed herein for the manufacture of a medicament for improving learning in a subject having mild dementia associated with Alzheimer's disease.

In one aspect, the disclosure provides a use of a pharmaceutical composition as disclosed herein for the manufacture of a medicament for improving learning and working memory in a subject having mild dementia associated with Alzheimer's disease.

In some embodiments, the total daily dose of Compound I is from about 0.3 mg to about 1.5 mg. In some embodiments, the total daily dose of Compound I is about 1.2 mg. In some embodiments, the total daily dose of Compound I is 1.2 mg. In some embodiments, the total daily dose of Compound I is about 0.9 mg. In some embodiments, the total daily dose of Compound I is 0.9 mg. In some embodiments, the total daily dose of Compound I is about 0.6 mg. In some embodiments, the total daily dose of Compound I is 0.6 mg. In some embodiments, the total daily dose of Compound I is about 0.3 mg. In some embodiments, the total daily dose of Compound I is 0.3 mg. In some embodiments, the treatment comprises administering at least one loading dose and at least one maintenance dose. In some embodiments, the treatment comprises administering the loading dose daily for at least 2, 4, 6, 8 or 10 weeks. In some embodiments, the treatment comprises administering the loading dose daily for at least 2 weeks. In some embodiments, the treatment comprises administering the loading dose daily for at least 4 weeks. In some embodiments, the treatment comprises administering the loading dose daily for at least 6 weeks. In some embodiments, the treatment comprises administering the loading dose daily for at least 8 weeks. In some embodiments, the treatment comprises administering the loading dose daily for at least 10 weeks. In some embodiments, the loading dose is about 1.2 mg daily of Compound I. In some embodiments, the loading dose is 1.2 mg daily of Compound I. In some embodiments, the maintenance dose is administered daily. In some embodiments, the maintenance dose is about 0.9 mg daily of Compound I. In some embodiments, the maintenance dose is 0.9 mg daily of Compound I. In some embodiments, the maintenance dose is about 0.6 mg daily of Compound I. In some embodiments, the maintenance dose is 0.6 mg daily of Compound I.

In one aspect, the disclosure provides a dosing regimen for administering a therapeutically effective amount of a pharmaceutical composition as disclosed herein to a subject in need thereof comprising administering to the subject at least one loading dose; and at least one maintenance dose. In some embodiments, the loading dose is administered daily for at least 2, 4, 6, 8 or 10 weeks. In some embodiments, the loading dose is administered daily for at least 2 weeks. In some embodiments, the loading dose is administered daily for at least 4 weeks. In some embodiments, the loading dose is administered daily for at least 6 weeks. In some embodiments, the loading dose is administered daily for at least 8 weeks. In some embodiments, the loading dose is administered daily for at least 10 weeks. In some embodiments, the loading dose is about 1.2 mg daily of Compound I. In some embodiments, the loading dose is 1.2 mg daily of Compound I. In some embodiments, the maintenance dose is about 0.9 mg daily of Compound I. In some embodiments, the maintenance dose is 0.9 mg daily of Compound I. In some embodiments, the maintenance dose is about 0.6 mg daily of Compound I. In some embodiments, the maintenance dose is 0.6 mg daily of Compound I.

### BRIEF DESCRIPTION OF THE FIGURES

FIG. 1A depicts an exemplary XRPD pattern for Form A.

FIG. 1B depicts exemplary TGA (upper) and DSC (lower) thermogram profiles for Form A.

FIG. 2A depicts an exemplary XRPD pattern for Form B.

FIG. 2B depicts exemplary TGA (upper) and DSC (lower) thermogram profiles for Form B.

FIG. 3A depicts an exemplary XRPD pattern for Form C.

FIG. 3B depicts exemplary TGA (upper) and DSC (lower) thermogram profiles for Form C.

FIG. 4A depicts an exemplary XRPD pattern for Form D.

FIG. 4B depicts exemplary TGA (upper) and DSC (lower) thermogram profiles for Form D.

FIG. 5A depicts an exemplary XRPD pattern for Form E.

FIG. 5B depicts exemplary TGA (upper) and DSC (lower) thermogram profiles for Form E.

### DETAILED DESCRIPTION OF THE INVENTION

### General Definitions

The term “herein” means the entire application.

Unless otherwise defined herein, scientific and technical terms used in this application shall have the meanings that are commonly understood by those of ordinary skill in the art to which this disclosure belongs. Generally, nomenclature used in connection with the compounds, composition and methods described herein, are those well-known and commonly used in the art.

It should be understood that any of the embodiments described herein, including those described under different embodiments of the disclosure and different parts of the specification (including embodiments described only in the Examples) can be combined with one or more other embodiments of the disclosure, unless explicitly disclaimed or improper. Combination of embodiments are not limited to those specific combinations claimed via the multiple dependent claims. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range and including the endpoints, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

All of the publications, patents and published patent applications referred to in this application are specifically incorporated by reference herein. In case of conflict, the present specification, including its specific definitions, will control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The terms “active ingredient,” “active agent” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient,” “active agent” and “active substance” may be an optically active isomer of a compound described herein.

The terms “drug,” and “therapeutic agent,” refer to a compound, or a pharmaceutical composition thereof, which is administered to a subject for treating, preventing, or ameliorating one or more symptoms of a condition, disorder, or disease.

The term “polymorph” as used herein refers to a crystalline form of a compound or a salt, hydrate, or solvate thereof, in a particular crystal packing arrangement. The term “polymorph” includes pseudopolymorphs, which are typically different solvates of a material, and thus their properties differ from one another. Thus, each distinct polymorph and pseudopolymorph disclosed herein is considered to be a distinct single crystalline form herein.

The term “crystalline,” as used herein, refers to a solid state form which consists of orderly arrangement of structural units. Different crystalline forms of the same compound, or a salt, hydrate, or solvate thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability, and solubility. See, e.g., Remington's Pharmaceutical Sciences, 18th ed., Mack Publishing, Easton Pa., 173 (1990); The United States Pharmacopeia, 23.sup.rd ed., 1843-1844 (1995) (incorporated herein by reference). As used herein, the terms “crystalline form,” “single crystalline form,” “crystalline solid form,” and “polymorph” are used interchangeably; the terms distinguish between crystals that have different properties (e.g., different XRPD patterns and/or different DSC scan results).

The term “substantially pure” relates to the composition of a specific crystalline solid form of Compound I that may be at least a particular weight percent free of impurities and/or other solid forms of Compound I. Particular weight percentages are 70%, 75%, 80%, 85%, 90%, 95%, 99%, or any percentage between 70% and 100%. In some embodiments, Compound I can be a substantially pure sample of any of the crystalline solid forms described herein. In some embodiments, Compound I can be substantially pure Form A. In some embodiments, Compound I can be substantially pure Form B. In some embodiments. In some embodiments, Compound I can be substantially pure Form D.

Crystalline forms are commonly characterized by X-ray powder diffraction (XRPD). An XRPD pattern of reflections (peaks, typically expressed in degrees 2-theta) is generally considered a fingerprint of a particular crystalline form. The relative intensities of the XRPD peaks may vary depending on, inter alia, the sample preparation technique, crystal size distribution, filters, the sample mounting procedure, and the particular instrument employed. In some instances, more (i.e., new peaks) or fewer (i.e., peaks may disappear) peaks may be present in the XRPD pattern, depending on the type of instrument or the settings. In some instances, any particular peak in an XRPD pattern may appear as a singlet, doublet, triplet, quartet, or multiplet, depending on the type of instrument or the settings, the sensitivity of the instrument, measuring conditions, and/or purity of the crystalline form. In some instances, any particular peak in an XRPD may appear in a symmetric shape or in an asymmetric shape, e.g., having a shoulder. Moreover, instrument variation and other factors can affect the 2-theta values. A skilled artisan understanding these variations is capable of identifying or ascertaining the defining features or characteristics of a particular crystal form using XRPD, as well as using other known physicochemical techniques.

The term “characteristic peaks” when referring to the peaks in an XRPD pattern of a crystalline form of Compound I refers to a collection of certain peaks whose values of 20 across a range of 0°-40° are, as a whole, uniquely assigned to one of the crystalline forms of Compound I.

The term “amorphous” as applied to a compound refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”).

The term “anhydrate” or “anhydrous” are used interchangeably and as applied to a compound refers to a solid state wherein the compound contains no structural water or other solvent molecule within the crystal lattice.

The term “solvate” refers to a compound provided herein or a salt thereof, which further includes a stoichiometric or non-stoichiometric amount of solvent bound by non-covalent intermolecular forces (e.g., hydrogen bonding). Where the solvent is water, the solvate is a hydrate. Where the solvent includes ethanol, the compound can be an ethanol solvate.

The term “stable” in the context of a polymorphic form disclosed herein refers to the stability of the polymorphic form relative to heat and/or humidity.

The term “pharmaceutically acceptable” refers to materials, compositions, and/or dosage forms which are approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans. The term also refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Pharmaceutically acceptable salt” refers to a salt of a compound disclosed herein that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

“Bioavailability” (% F) refers to the percentage of Compound I dosed that is delivered into the general circulation of the animal or human being studied. The AUC (from zero to infinity) represents the total drug exposure across time. The total exposure (AUC_{Inf} or AUC_{∞}) of a drug when administered intravenously is usually defined as 100% bioavailable (F %).

“Oral bioavailability” refers to the extent to which Compound I is absorbed into the general circulation when the pharmaceutical composition is taken orally as compared to intravenous injection.

The term “geometric mean” refers to the average value or mean which signifies the central tendency of the set of numbers by finding the product of their values. The formula to calculate the geometric mean is GM=(X₁·X₂· . . . Xₙ)^{1/n}.

“Plasma concentration” refers to the concentration of Compound 1 in the plasma component of blood of a subject. It is understood that the plasma concentration of Compound I may vary significantly between subjects, due to variability with respect to metabolism and/or possible interactions with other therapeutic agents. In accordance with one embodiment disclosed herein, the plasma concentration of Compound 1 may vary from subject to subject. Likewise, values such as maximum plasma concentration (Cₘₐₓ) or time to reach maximum plasma concentration (tₘₐₓ), or total area under the plasma concentration time curve (AUC_{∞}) may vary from subject to subject.

The term “AUCₗₐₛₜ” refers to the area under the plasma concentration-time curve from time 0 to tₗₐₛₜ.

The term “tₗₐₛₜ” refers to the time of last measurable concentration.

The term “tₘₐₓ” refers to the time of maximum concentration.

The term “Cₘₐₓ” refers to the maximum drug concentration in the plasma.

The term “CL_{z}/F” refers to the apparent clearance unadjusted for bioavailability measured after extravascular dosing.

The term “V_{z}/F” refers to the apparent volume of distribution unadjusted for bioavailability.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

A “subject” to which administration is contemplated includes, but is not limited to, human subject (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, the term “treat,” “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation, amelioration, reduction of the severity, or slowing the progression, of one or more symptoms or conditions associated with a condition, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Exemplary beneficial clinical results are described herein.

As used herein, and unless otherwise specified, the term “prophylactic,” “prevention” and variations thereof, contemplates an action that occurs before a subject begins to suffer from the specified disease, disorder, or condition.

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

The terms “pharmaceutically effective amount,” “therapeutically effective amount,” or “therapeutically effective dose” refer to an amount sufficient to treat a disease in a patient, e.g., effecting a beneficial and/or desirable alteration in the health of a patient suffering from a disease, treatment, healing, inhibition or amelioration of a physiological response or condition, delaying or minimizing one or more symptoms associated with the disease, disorder or condition etc. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of disease, the therapeutics or combination of therapeutics selected for administration, and the mode of administration. The skilled worker can readily determine the effective amount for a given situation by routine experimentation. The terms “pharmaceutically effective amount,” “therapeutically effective amount,” or “therapeutically effective dose” also refer to the amount required to improve the clinical symptoms of a patient. A therapeutically effective amount of a compound also refers to an amount of the therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

A “loading dose” is an initial higher dose of a drug that may be given at the beginning of a course of treatment to rapidly achieve a therapeutic concentration in the body before dropping down to a lower maintenance dose.

A “maintenance dose” as used herein refers to a lower dose of a drug administered following the loading dose.

As used herein, and unless otherwise specified, “pharmacokinetics” can be defined as the study of bodily absorption, distribution, metabolism, and excretion of drugs. “Pharmacokinetics” can also be defined as the characteristic interactions of a drug and a body in terms of its absorption, distribution, metabolism, and excretion; or a branch of pharmacology concerned with the way drugs are taken into, move around, and are eliminated from, a body.

“Administering” or “administration of” a substance, a compound or an agent to a subject can be carried out using one of a variety of methods known to those skilled in the art. For example, a compound or an agent can be administered, e.g., as a soft gel, hardshell, or oral liquid. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. When a method is part of a therapeutic regimen involving more than one agent or treatment modality, the disclosure contemplates that the agents may be administered at the same or differing times and via the same or differing routes of administration. Appropriate methods of administering a substance, a compound or an agent to a subject will also depend, for example, on the age of the subject, whether the subject is active or inactive at the time of administering, whether the subject is cognitively impaired at the time of administering, the extent of the impairment, and the chemical and biological properties of the compound or agent (e.g., solubility, digestibility, bioavailability, stability and toxicity).

### Pharmaceutical Compositions

In another aspect, described herein is a lipid formulation comprising Compound I or a pharmaceutically acceptable salt thereof and methods of use thereof for treating a disease or disorder associated with NMDA receptor modulation. These lipid formulations provide several advantages including a) having equivalent exposure in high fat, low fat/calorie, and fasting prandial states, b) low exposure variability c) increased exposure, AUC, of drug product versus tablet in all prandial states, and d) increased Cₘₐₓ of drug product versus tablet in all prandial states. The lipid formulations disclosed herein allow for self-emulsification of the formulation when exposed to gastrointestinal fluids. This ensures that Compound I and vehicle are uniformly dispersed allowing for consistent uptake of Compound I under a number of different fed/fasted conditions.

Therefore, in one aspect, the disclosure provides a pharmaceutical composition comprising Compound I:

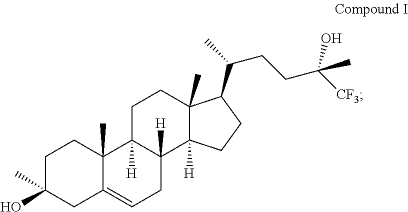

or a pharmaceutically acceptable salt thereof, and one or more glycerides, wherein said glycerides comprise about 30% to about 100% w/w monoglycerides.

The pharmaceutical composition disclosed herein may be prepared using any form of Compound I, including, e.g., the free base, salt thereof or crystalline forms (e.g., hydrate, anhydrate and solvate forms). For the sake of clarity, for example when a crystalline form of Compound I is used to prepare a pharmaceutical composition as disclosed herein, in the formulation, Compound I is solubilized such that there are no crystals of Compound I in the pharmaceutical composition. Similarly, when a pharmaceutically acceptable salt of Compound I is used to prepare a pharmaceutical composition as disclosed herein, in the pharmaceutical composition, the salt form of Compound I is fully dissolved.

In some embodiments, the disclosure provides a pharmaceutical composition comprising Compound I:

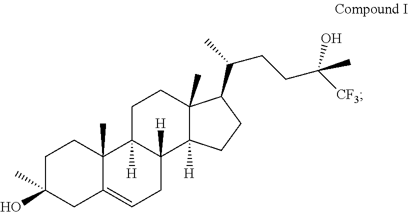

and one or more glycerides, said glycerides comprise about 30% to about 100% w/w monoglycerides. In some embodiments, the pharmaceutical composition is prepared using Compound I. In other embodiments, the pharmaceutical composition is prepared using a pharmaceutically acceptable salt of Compound I. Compound I and its chemical synthesis are disclosed in U.S. patent Ser. No. 10/227,375 and/PCT Publication No. WO2014160480, the disclosures of which are incorporated by reference herein.

In some embodiments, the pharmaceutical composition is prepared using a crystalline form of Compound I. In some embodiments, the crystalline form of Compound I is an anhydrate. In some embodiments, the crystalline form of Compound I is a solvate. In some embodiments, the crystalline form of Compound I is a hydrate. In some embodiments, the crystalline form of Compound I is a monohydrate. In some embodiments, Compound I used to prepare the pharmaceutical composition disclosed herein is a crystalline form selected from Form A, Form B, Form C, Form D and Form E. In some embodiments, the crystalline form is Form A. In some embodiments, the crystalline form is Form B. In some embodiments, the crystalline form is Form C. In some embodiments, the crystalline form is Form D. In some embodiments, the crystalline form is Form E. The preparation of crystalline Forms A, B, C, D and E is disclosed in WO2023250185 and WO2024015201, the disclosures of which are incorporated by reference herein.

### A. Crystalline Form A

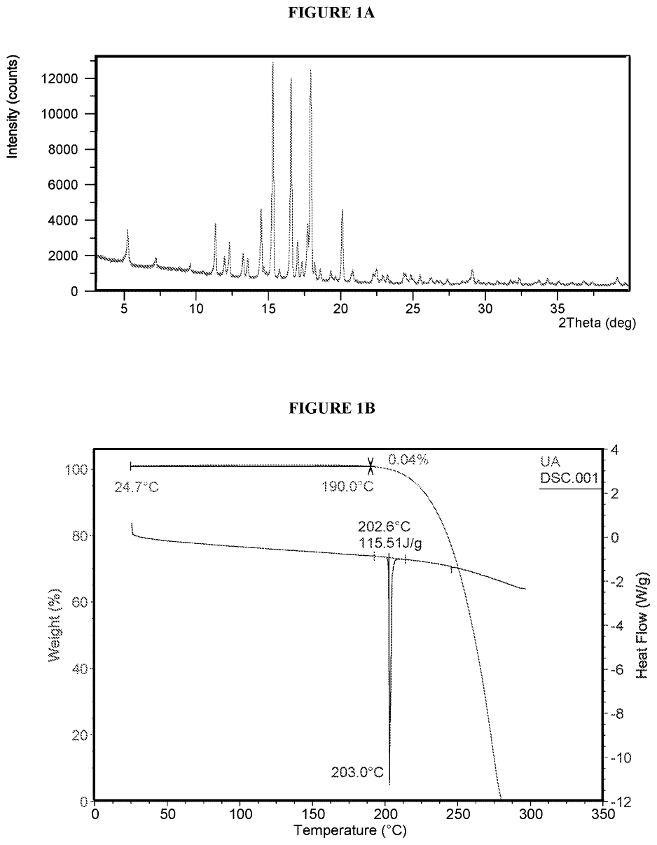

In some embodiments, the crystalline form of Compound I used in the preparation of the pharmaceutical composition as disclosed herein is crystalline Form A of Compound I. In some embodiments, the anhydrous crystalline form of Compound I is Form A. In some embodiments, crystalline Form A has one or more characteristics selected from the group consisting of:
a) an XRPD pattern comprising peaks at 15.3±0.2, 16.6±0.2, and 17.9±0.2 degrees 2θ;
b) an XRPD pattern substantially as shown in FIG. 1A;
c) a DSC thermogram profile comprising an endothermic peak at about 203° C.;
d) a DSC thermogram profile substantially as shown in FIG. 1B;
e) a TGA profile exhibiting a loss of at least about 0.04 wt % between about 25° C. and about 190° C.; and
f) a TGA profile substantially as shown in FIG. 1B.

In some embodiments, Form A is characterized by an XRPD pattern comprising peaks at 15.3±0.2, 16.6±0.2, and 17.9±0.2 degrees 2θ. In some embodiments, Form A is characterized by an XRPD pattern comprising peaks at 15.3±0.2, 16.6±0.2, and 17.9±0.2 degrees 2θ, and further comprising at least one additional peak selected from the group consisting of 11.3±0.2, 14.5±0.2, 17.7±0.2, and 20.1±0.2 degrees 2θ. In some embodiments, Form A is characterized by an XRPD pattern comprising peaks at 14.5±0.2, 15.3±0.2, 16.6±0.2, 17.9±0.2, and 20.1±0.2 degrees 2θ. In some embodiments, Form A is characterized by an XRPD pattern comprising peaks at 11.3±0.2, 14.5±0.2, 15.3±0.2, 16.6±0.2, 17.7±0.2, 17.9±0.2, and 20.1±0.2 degrees 2θ. In some embodiments, crystalline Form A is characterized by an XRPD pattern substantially as depicted in FIG. 1A.

In some embodiments, Form A is characterized by a thermo-gravimetric analysis (TGA) thermogram exhibiting that Form A loses at least about 0.04 wt % between about 25° C. and about 190° C. In some embodiments, Form A is characterized by a thermo-gravimetric analysis (TGA) thermogram exhibiting that Form A loses about 0.04 wt % between about 25° C. and about 190° C. In some embodiments, Form A is characterized by a thermo-gravimetric analysis (TGA) thermogram substantially as depicted in FIG. 1B.

In some embodiments, Form A is characterized by a differential scanning calorimetry (DSC) thermogram substantially as depicted in FIG. 1B. In some embodiments, Form A is characterized by a DSC thermogram comprising an endothermic peak at about 203° C. In some embodiments, Form A is characterized by a DSC thermogram comprising a melting endothermic peak at about 203° C.

In some embodiments, Form A is substantially free of other polymorphic forms. In some embodiments, Form A has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form A has a polymorphic purity of at least 90%. In some embodiments, Form A has a polymorphic purity of at least 92%. In some embodiments, Form A has a polymorphic purity of at least 94%. In some embodiments, Form A has a polymorphic purity of at least 96%. In some embodiments, Form A has a polymorphic purity of at least 98%. In some embodiments, Form A has a polymorphic purity of at least 99%.

### B. Crystalline Form B

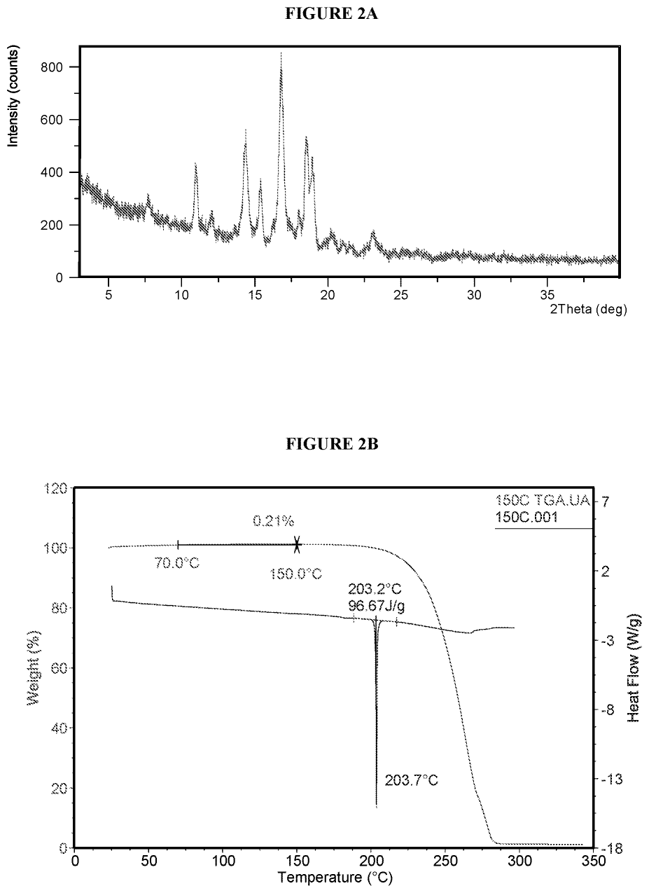

In some embodiments, the crystalline form of Compound I used in the preparation of the pharmaceutical composition as disclosed herein is crystalline Form B of Compound I. In some embodiments, the anhydrous crystalline form of Compound I is Form B. In some embodiments Form B has one or more characteristics selected from the group consisting of:
a) an XRPD pattern comprising peaks at 14.4±0.2, 16.8±0.2, and 18.5±0.2 degrees 2θ;
b) an XRPD pattern substantially as shown in FIG. 2A;
c) a DSC thermogram profile comprising an endothermic peak at about 204° C.;
d) a DSC thermogram profile substantially as shown in FIG. 2B;
e) a TGA profile exhibiting a loss of at least about 0.2 wt % between about 70° C. and about 150° C.; and
f) a TGA profile substantially as shown in FIG. 2B.

In some embodiments, Form B is characterized by an XRPD pattern comprising peaks at 14.4±0.2, 16.8±0.2, and 18.5±0.2 degrees 2θ. In some embodiments, Form B is characterized by an XRPD pattern comprising peaks at 14.4±0.2, 16.8±0.2, and 18.5±0.2 degrees 2θ, and further comprising at least one additional peak selected from the group consisting of 10.9±0.2, 15.4±0.2, and 19.0±0.2 degrees 2θ. In some embodiments, Form B is characterized by an XRPD pattern comprising peaks at 10.9±0.2, 14.4±0.2, 16.8±0.2, 18.5±0.2 and 19.0±0.2 degrees 2θ. In some embodiments, Form B is characterized by an XRPD pattern comprising peaks at 10.9±0.2, 14.4±0.2, 15.4±0.2, 16.8±0.2, 18.5±0.2 and 19.0±0.2 degrees 2θ. In some embodiments, crystalline Form B is characterized by an XRPD pattern substantially as depicted in FIG. 2A.

In some embodiment, Form B is characterized by a thermo-gravimetric analysis (TGA) thermogram exhibiting that Form B loses at least about 0.2 wt % between about 70° C. and about 150° C. In some embodiments, Form B is characterized by a thermo-gravimetric analysis (TGA) thermogram substantially as depicted in FIG. 2B.

In some embodiments, Form B is characterized by a differential scanning calorimetry (DSC) thermogram substantially as depicted in FIG. 2B. In some embodiments, Form B is characterized by a DSC thermogram comprising an endothermic peak at about 204° C. In some embodiments, Form B is characterized by a DSC thermogram comprising a melting endothermic peak at about 204° C.

In some embodiments, Form B is substantially free of other polymorphic forms. In some embodiment, Form B has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form B has a polymorphic purity of at least 90%. In some embodiments, Form B has a polymorphic purity of at least 92%. In some embodiments, Form B has a polymorphic purity of at least 94%. In some embodiments, Form B has a polymorphic purity of at least 96%. In some embodiments, Form B has a polymorphic purity of at least 98%. In some embodiments, Form B has a polymorphic purity of at least 99%.

### C. Crystalline Form C

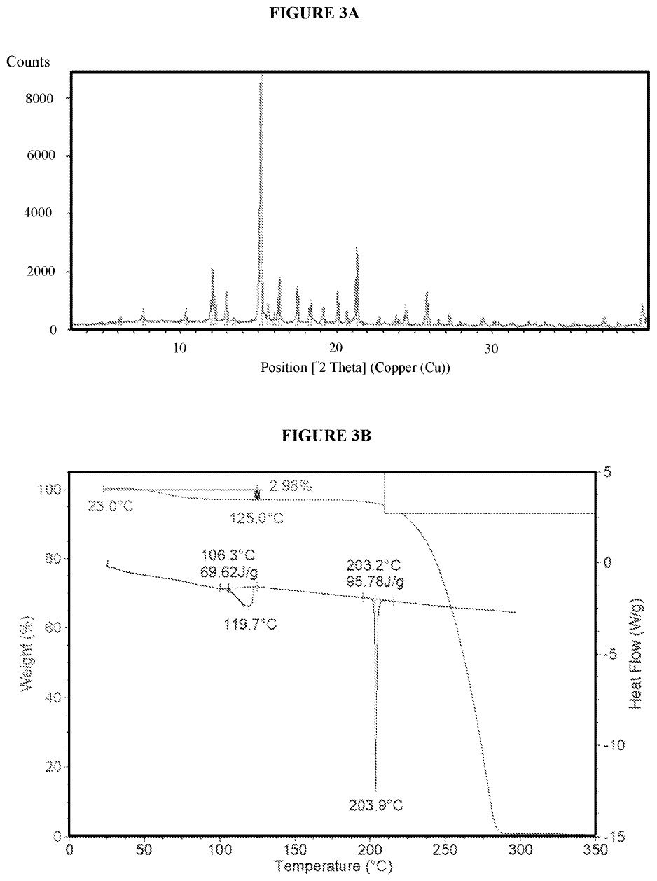

In some embodiments, the crystalline form of Compound I used in the preparation of the pharmaceutical composition as disclosed herein is crystalline Form C of Compound I. In some embodiments, the hydrate crystalline form of Compound I is Form C. In some embodiments, the monohydrate crystalline form of Compound I is Form C. In some embodiments Form C has one or more characteristics selected from the group consisting of:
a) an XRPD pattern comprising peaks at 12.0±0.2, 15.1±0.2, and 21.3±0.2 degrees 2θ;
b) an XRPD pattern substantially as shown in FIG. 3A;
c) a DSC thermogram profile comprising an endothermic peak at about 204° C.;
d) a DSC thermogram profile comprising an endothermic peak at about 120° C.;
e) a DSC thermogram profile substantially as shown in FIG. 3B;
f) a TGA profile exhibiting a loss of at least about 3 wt % between about 23° C. and about 125° C.; and
g) a TGA profile substantially as shown in FIG. 3B.

In some embodiments, Form C is characterized by an XRPD pattern comprising peaks at 12.0±0.2, 15.1±0.2, and 21.3±0.2 degrees 2θ. In some embodiments, Form C is characterized by an XRPD pattern comprising peaks at 12.0±0.2, 15.1±0.2, and 21.3±0.2 degrees 2θ, and further comprising at least one additional peak selected from the group consisting of 12.9±0.2, 16.3±0.2, 17.5±0.2, 20.1±0.2, and 25.8±0.2, degrees 2θ. In some embodiments, Form C is characterized by an XRPD pattern comprising peaks at 12.0 0.2, 15.1 0.2, 16.3 0.2, 17.5±0.2, 20.1±0.2, 21.3±0.2, and 25.8±0.2 degrees 2θ. In some embodiments, Form C is characterized by an XRPD pattern comprising peaks at 12.0±0.2, 12.9±0.2, 15.1±0.2, 16.3±0.2, 17.5±0.2, 20.1±0.2, 21.3±0.2, and 25.8±0.2, degrees 2θ. In some embodiments, crystalline Form C is characterized by an XRPD pattern substantially as depicted in FIG. 3A.

In some embodiments, Form C is characterized by a thermo-gravimetric analysis (TGA) thermogram exhibiting that Form C loses at least about 3 wt % between about 23° C. and about 125° C. In some embodiments, Form C is characterized by a thermo-gravimetric analysis (TGA) thermogram exhibiting that Form C loses about 3-4 wt % between about 23° C. and about 125° C. In some embodiments, Form C is characterized by a thermo-gravimetric analysis (TGA) thermogram substantially as depicted in FIG. 3B.

In some embodiments, Form C is characterized by a differential scanning calorimetry (DSC) thermogram substantially as depicted in FIG. 3B. In some embodiments, Form C is characterized by a DSC thermogram comprising an endothermic peak at about 204° C. In some embodiments, Form C is characterized by a DSC thermogram comprising an endothermic peak at about 120° C. In some embodiments, Form C is characterized by a DSC thermogram comprising an endothermic peak at about 120° C. and about 204° C. In some embodiments, Form C is characterized by a DSC thermogram comprising a dehydration endothermic peak at about 120° C. In some embodiments, Form C is characterized by a DSC thermogram comprising a melting endothermic peak at about 204° C.

In some embodiments, Form C is substantially free of other polymorphic forms. In some embodiments, Form C has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form C has a polymorphic purity of at least 90%. In some embodiments, Form C has a polymorphic purity of at least 92%. In some embodiments, Form C has a polymorphic purity of at least 94%. In some embodiments, Form C has a polymorphic purity of at least 96%. In some embodiments, Form C has a polymorphic purity of at least 98%. In some embodiments, Form C has a polymorphic purity of at least 99%.

### D. Crystalline Form D

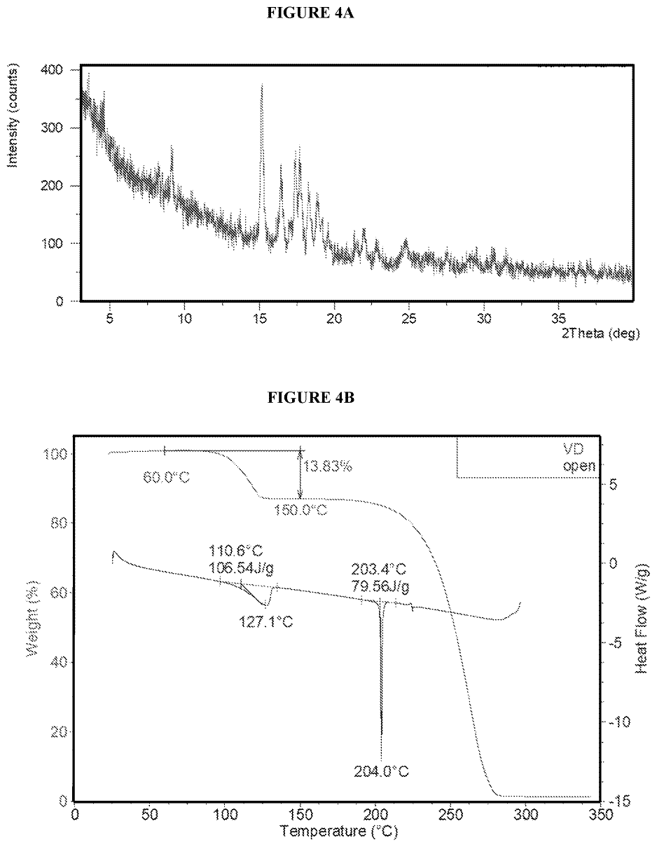

In some embodiments, the crystalline form of Compound I used in the preparation of the pharmaceutical composition as disclosed herein is crystalline Form D of Compound I. In some embodiments, the monohydrate crystalline form of Compound I is Form D. In some embodiments, Form D has one or more characteristics selected from the group consisting of:
a) an XRPD pattern comprising peaks at 15.2±0.2, 17.4±0.2 and 17.7±0.2 degrees 2θ;
b) an XRPD pattern substantially as shown in FIG. 4A;
c) a DSC thermogram profile comprising an endothermic peak at about 204° C.;
d) a DSC thermogram profile comprising an endothermic peak at about 127° C.;
e) a DSC thermogram profile substantially as shown in FIG. 4B;
f) a TGA profile exhibiting a loss of at least about 14 wt % between about 60° C. and about 150° C.; and
g) a TGA profile substantially as shown in FIG. 4B.

In some embodiments, Form D is characterized by an XRPD pattern comprising peaks at 15.2±0.2, 17.4±0.2 and 17.7±0.2 degrees 2θ. In some embodiments, Form D is characterized by an XRPD pattern comprising peaks at 15.2±0.2, 17.4±0.2 and 17.7±0.2 degrees 2θ, and further comprising at least one additional peak selected from the group consisting of 9.1±0.2, 16.4±0.2, 18.3±0.2, and 18.9±0.2 degrees 2θ. In some embodiments, Form D is characterized by an XRPD pattern comprising peaks at 15.2±0.2, 16.4±0.2, 17.4±0.2, 17.7±0.2, 18.3±0.2, and 18.9±0.2 degrees 2θ. In some embodiments, Form D is characterized by an XRPD pattern comprising peaks at 9.1±0.2, 15.2±0.2, 16.4±0.2, 17.4±0.2, 17.7±0.2, 18.3±0.2, and 18.9±0.2 degrees 2θ. In some embodiments, crystalline Form D is characterized by an XRPD pattern substantially as depicted in FIG. 4A.

In some embodiments, Form D is characterized by a thermo-gravimetric analysis (TGA) thermogram exhibiting that Form D loses at least about 14 wt % between about 60° C. and about 150° C. In some embodiments, Form D is characterized by a thermo-gravimetric analysis (TGA) thermogram exhibiting that Form D loses about 14-15 wt % between about 60° C. and about 150° C. In some embodiments, Form D is characterized by a thermo-gravimetric analysis (TGA) thermogram exhibiting that Form D loses about 14 wt % between about 60° C. and about 150° C. In some embodiments, Form D is characterized by a thermo-gravimetric analysis (TGA) thermogram substantially as depicted in FIG. 4B.

In some embodiments, Form D is characterized by a differential scanning calorimetry (DSC) thermogram substantially as depicted in FIG. 4B. In some embodiments, Form D is characterized by a DSC thermogram comprising an endothermic peak at about 204° C. In some embodiments, Form D is characterized by a DSC thermogram comprising an endothermic peak at about 127° C. In some embodiments, Form D is characterized by a DSC thermogram comprising an endothermic peak at about 127° C. and about 204° C. In some embodiments, Form D is characterized by a DSC thermogram comprising a desolvation endothermic peak at about 127° C. In some embodiments, Form D is characterized by a DSC thermogram comprising a melting endothermic peak at about 204° C.

In some embodiments, Form D is substantially free of other polymorphic forms. In some embodiments, Form D has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form D has a polymorphic purity of at least 90%. In some embodiments, Form D has a polymorphic purity of at least 92%. In some embodiments, Form D has a polymorphic purity of at least 94%. In some embodiments, Form D has a polymorphic purity of at least 96%. In some embodiments, Form D has a polymorphic purity of at least 98%. In some embodiments, Form D has a polymorphic purity of at least 99%.

### D. Crystalline Form E

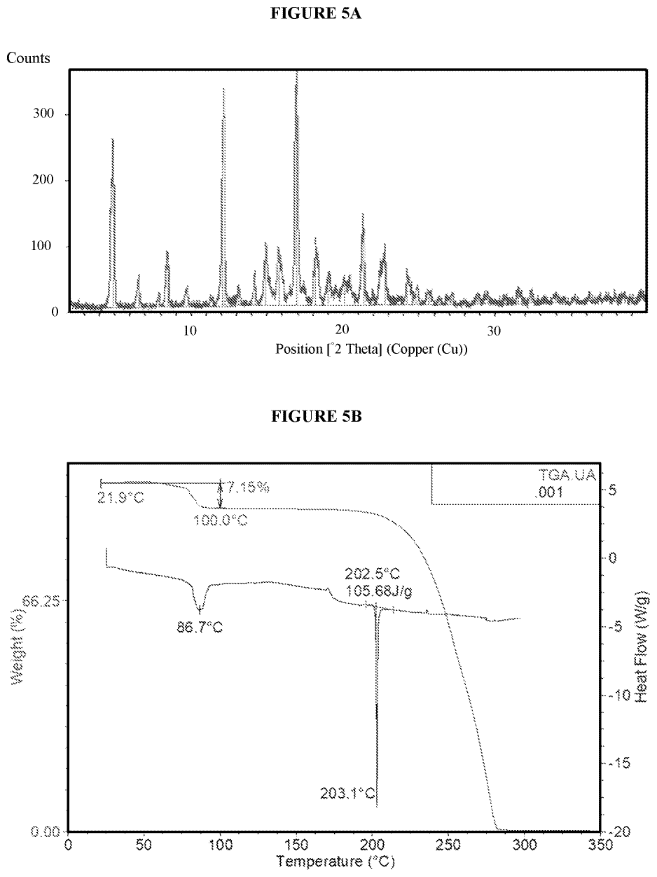

In some embodiments, the crystalline form is a n-BuOH solvate. In some embodiments, the crystalline form is crystalline Form E characterized by an XRPD pattern comprising peaks at 4.9±0.2, 12.0±0.2, 12.2±0.2, and 17.0±0.2 degrees 2θ. In some embodiments, the XRPD pattern of crystalline Form E further comprises at least one additional peak at a position selected from the group consisting of at 8.5±0.2, 14.9±0.2, 15.8±0.2, 18.2±0.2 and 21.4±0.2 degrees 2θ. In some embodiments, the crystalline form is crystalline Form E characterized by having an XRPD pattern with characteristic peaks at 4.9±0.2, 12.0±0.2, 12.2±0.2, 17.0±0.2, and 21.4±0.2 degrees 2θ. In some embodiments, the crystalline form is crystalline Form E characterized by an XRPD pattern substantially as depicted in FIG. 5A.

In some embodiments, the crystalline Form E has a TGA thermogram substantially as depicted in FIG. 5B. In some embodiments, the crystalline Form E has a differential scanning calorimetry (DSC) thermogram substantially as depicted in FIG. 5B. In some embodiments, the thermogram comprises an endothermic peak at about 203° C. In some embodiments, the DSC thermogram comprises an endothermic peak at about 87° C.

In some embodiments, Form E is substantially free of other polymorphic forms. In some embodiments, Form E has a polymorphic purity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, Form E has a polymorphic purity of at least 90%. In some embodiments, Form E has a polymorphic purity of at least 92%. In some embodiments, Form D has a polymorphic purity of at least 94%. In some embodiments, Form E has a polymorphic purity of at least 96%. In some embodiments, Form E has a polymorphic purity of at least 98%. In some embodiments, Form E has a polymorphic purity of at least 99%.

In some embodiments, the pharmaceutical composition comprises about 0.05% to about 1.0% w/w of Compound I or a pharmaceutically acceptable salt thereof. In some embodiments, the pharmaceutical composition comprises about 0.1% to about 1.0% w/w of Compound I or a pharmaceutically acceptable salt thereof. In some embodiments, the pharmaceutical composition comprises about 0.15% to about 1.0% w/w of Compound I or a pharmaceutically acceptable salt thereof. In some embodiments, the pharmaceutical composition comprises about 0.2% to about 1.0% w/w of Compound I or a pharmaceutically acceptable salt thereof. In some embodiments, the pharmaceutical composition comprises about 0.25% to about 1.0% w/w of Compound I or a pharmaceutically acceptable salt thereof. In some embodiments, the pharmaceutical composition comprises about 0.3% to about 1.0% w/w of Compound I or a pharmaceutically acceptable salt thereof. In some embodiments, the pharmaceutical composition comprises about 0.35% to about 1.0% w/w of Compound I or a pharmaceutically acceptable salt thereof. In some embodiments, the pharmaceutical composition comprises about 0.4% to about 1.0% w/w of Compound I or a pharmaceutically acceptable salt thereof. In some embodiments, the pharmaceutical composition comprises about 0.45% to about 1.0% w/w of Compound I or a pharmaceutically acceptable salt thereof. In some embodiments, the pharmaceutical composition comprises about 0.5% to about 1.0% w/w of Compound I or a pharmaceutically acceptable salt thereof. In some embodiments, the pharmaceutical composition comprises about 0.55% to about 1.0% w/w of Compound I or a pharmaceutically acceptable salt thereof. In some embodiments, the pharmaceutical composition comprises about 0.6% to about 1.0% w/w of Compound I or a pharmaceutically acceptable salt thereof. In some embodiments, the pharmaceutical composition comprises about 0.65% to about 1.0% w/w of Compound I or a pharmaceutically acceptable salt thereof. In some embodiments, the pharmaceutical composition comprises about 0.7% to about 1.0% w/w of Compound I or a pharmaceutically acceptable salt thereof. In some embodiments, the pharmaceutical composition comprises about 0.75% to about 1.0% w/w of Compound I or a pharmaceutically acceptable salt thereof. In some embodiments, the pharmaceutical composition comprises about 0.8% to about 1.0% w/w of Compound I or a pharmaceutically acceptable salt thereof. In some embodiments, the pharmaceutical composition comprises about 0.85% to about 1.0% w/w of Compound I or a pharmaceutically acceptable salt thereof. In some embodiments, the pharmaceutical composition comprises about 0.9% to about 1.0% w/w of Compound I or a pharmaceutically acceptable salt thereof. In some embodiments, the pharmaceutical composition comprises about 0.95% to about 1.0% w/w of Compound I or a pharmaceutically acceptable salt thereof. In some embodiments, the pharmaceutical composition comprises about 0.1% to about 0.9% w/w of Compound I or a pharmaceutically acceptable salt thereof. In some embodiments, the pharmaceutical composition comprises about 0.1% to about 0.8% w/w of Compound I or a pharmaceutically acceptable salt thereof. In some embodiments, the pharmaceutical composition comprises about 0.1% to about 0.7% w/w of Compound I or a pharmaceutically acceptable salt thereof. In some embodiments, the pharmaceutical composition comprises about 0.1% to about 0.6% w/w of Compound I or a pharmaceutically acceptable salt thereof. In some embodiments, the pharmaceutical composition comprises about 0.1% to about 0.5% w/w of Compound I or a pharmaceutically acceptable salt thereof. In some embodiments, the pharmaceutical composition comprises about 0.1% to about 0.4% w/w of Compound I or a pharmaceutically acceptable salt thereof. In some embodiments, the pharmaceutical composition comprises about 0.1% to about 0.3% w/w of Compound I or a pharmaceutically acceptable salt thereof. In some embodiments, the pharmaceutical composition comprises about 0.1% to about 0.2% w/w of Compound I or a pharmaceutically acceptable salt thereof. In some embodiments, the pharmaceutical composition comprises about 0.2% to about 0.4% w/w of Compound I or a pharmaceutically acceptable salt thereof. In some embodiments, the pharmaceutical composition comprises about 0.25% to about 0.4% w/w Compound I or a pharmaceutically acceptable salt thereof. In some embodiments, the pharmaceutical composition comprises about 0.3% to about 0.4% w/w Compound I or a pharmaceutically acceptable salt thereof. In some embodiments, the pharmaceutical composition comprises about 0.3% to about 0.35% w/w Compound I or a pharmaceutically acceptable salt thereof. In some embodiments, the pharmaceutical composition comprises about 0.25% to about 0.35% w/w Compound I or a pharmaceutically acceptable salt thereof. In some embodiments, the pharmaceutical composition comprises about 0.25% to about 0.3% w/w Compound I or a pharmaceutically acceptable salt thereof. In some embodiments, the pharmaceutical composition comprises about 0.25% w/w Compound I or a pharmaceutically acceptable salt thereof. In some embodiments, the pharmaceutical composition comprises about 0.3% w/w Compound I or a pharmaceutically acceptable salt thereof. In some embodiments, the pharmaceutical composition comprises about 0.35% w/w Compound I or a pharmaceutically acceptable salt thereof. In some embodiments, the pharmaceutical composition comprises about 0.4% w/w Compound I or a pharmaceutically acceptable salt thereof. In some embodiments, the pharmaceutical composition comprises about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1% of Compound I or a pharmaceutically acceptable salt thereof.

In some embodiments, the pharmaceutical composition comprises 0.05% to 1.0% w/w of Compound I or a pharmaceutically acceptable salt thereof. In some embodiments, the pharmaceutical composition comprises 0.1% to 1.0% w/w of Compound I or a pharmaceutically acceptable salt thereof. In some embodiments, the pharmaceutical composition comprises 0.15% to 1.0% w/w of Compound I or a pharmaceutically acceptable salt thereof. In some embodiments, the pharmaceutical composition comprises 0.2% to 1.0% w/w of Compound I or a pharmaceutically acceptable salt thereof. In some embodiments, the pharmaceutical composition comprises 0.25% to 1.0% w/w of Compound I or a pharmaceutically acceptable salt thereof. In some embodiments, the pharmaceutical composition comprises 0.3% to 1.0% w/w of Compound I or a pharmaceutically acceptable salt thereof. In some embodiments, the pharmaceutical composition comprises 0.35% to 1.0% w/w of Compound I or a pharmaceutically acceptable salt thereof. In some embodiments, the pharmaceutical composition comprises 0.4% to 1.0% w/w of Compound I or a pharmaceutically acceptable salt thereof. In some embodiments, the pharmaceutical composition comprises 0.45% to 1.0% w/w of Compound I or a pharmaceutically acceptable salt thereof. In some embodiments, the pharmaceutical composition comprises 0.5% to 1.0% w/w of Compound I or a pharmaceutically acceptable salt thereof. In some embodiments, the pharmaceutical composition comprises 0.55% to 1.0% w/w of Compound I or a pharmaceutically acceptable salt thereof. In some embodiments, the pharmaceutical composition comprises 0.6% to 1.0% w/w of Compound I or a pharmaceutically acceptable salt thereof. In some embodiments, the pharmaceutical composition comprises 0.65% to 1.0% w/w of Compound I or a pharmaceutically acceptable salt thereof. In some embodiments, the pharmaceutical composition comprises 0.7% to 1.0% w/w of Compound I or a pharmaceutically acceptable salt thereof. In some embodiments, the pharmaceutical composition comprises 0.75% to 1.0% w/w of Compound I or a pharmaceutically acceptable salt thereof. In some embodiments, the pharmaceutical composition comprises 0.8% to 1.0% w/w of Compound I or a pharmaceutically acceptable salt thereof. In some embodiments, the pharmaceutical composition comprises 0.85% to 1.0% w/w of Compound I or a pharmaceutically acceptable salt thereof. In some embodiments, the pharmaceutical composition comprises 0.9% to 1.0% w/w of Compound I or a pharmaceutically acceptable salt thereof. In some embodiments, the pharmaceutical composition comprises 0.95% to 1.0% w/w of Compound I or a pharmaceutically acceptable salt thereof. In some embodiments, the pharmaceutical composition comprises 0.1% to 0.9% w/w of Compound I or a pharmaceutically acceptable salt thereof. In some embodiments, the pharmaceutical composition comprises 0.1% to 0.8% w/w of Compound I or a pharmaceutically acceptable salt thereof. In some embodiments, the pharmaceutical composition comprises 0.1% to 0.7% w/w of Compound I or a pharmaceutically acceptable salt thereof. In some embodiments, the pharmaceutical composition comprises 0.1% to 0.6% w/w of Compound I or a pharmaceutically acceptable salt thereof. In some embodiments, the pharmaceutical composition comprises 0.1% to 0.5% w/w of Compound I or a pharmaceutically acceptable salt thereof. In some embodiments, the pharmaceutical composition comprises 0.1% to 0.4% w/w of Compound I or a pharmaceutically acceptable salt thereof. In some embodiments, the pharmaceutical composition comprises 0.1% to 0.3% w/w of Compound I or a pharmaceutically acceptable salt thereof. In some embodiments, the pharmaceutical composition comprises 0.1% to 0.2% w/w of Compound I or a pharmaceutically acceptable salt thereof. In some embodiments, the pharmaceutical composition comprises 0.2% to 0.4% w/w of Compound I or a pharmaceutically acceptable salt thereof. In some embodiments, the pharmaceutical composition comprises 0.25% to 0.4% w/w Compound I or a pharmaceutically acceptable salt thereof. In some embodiments, the pharmaceutical composition comprises 0.3% to 0.4% w/w Compound I or a pharmaceutically acceptable salt thereof. In some embodiments, the pharmaceutical composition comprises 0.3% to 0.35% w/w Compound I or a pharmaceutically acceptable salt thereof. In some embodiments, the pharmaceutical composition comprises 0.25% to 0.35% w/w Compound I or a pharmaceutically acceptable salt thereof. In some embodiments, the pharmaceutical composition comprises 0.25% to 0.3% w/w Compound I or a pharmaceutically acceptable salt thereof. In some embodiments, the pharmaceutical composition comprises 0.25% w/w Compound I or a pharmaceutically acceptable salt thereof. In some embodiments, the pharmaceutical composition comprises 0.3% w/w Compound I or a pharmaceutically acceptable salt thereof. In some embodiments, the pharmaceutical composition comprises 0.35% w/w Compound I or a pharmaceutically acceptable salt thereof. In some embodiments, the pharmaceutical composition comprises 0.4% w/w Compound I or a pharmaceutically acceptable salt thereof. In some embodiments, the pharmaceutical composition comprises 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1% of Compound I or a pharmaceutically acceptable salt thereof.

In some embodiments, the pharmaceutical composition comprises about 0.05% to about 1.0% w/w of Compound I. In some embodiments, the pharmaceutical composition comprises about 0.1% to about 1.0% w/w of Compound I. In some embodiments, the pharmaceutical composition comprises about 0.15% to about 1.0% w/w of Compound I. In some embodiments, the pharmaceutical composition comprises about 0.2% to about 1.0% w/w of Compound I. In some embodiments, the pharmaceutical composition comprises about 0.25% to about 1.0% w/w of Compound I. In some embodiments, the pharmaceutical composition comprises about 0.3% to about 1.0% w/w of Compound I. In some embodiments, the pharmaceutical composition comprises about 0.35% to about 1.0% w/w of Compound I. In some embodiments, the pharmaceutical composition comprises about 0.4% to about 1.0% w/w of Compound I. In some embodiments, the pharmaceutical composition comprises about 0.45% to about 1.0% w/w of Compound I. In some embodiments, the pharmaceutical composition comprises about 0.5% to about 1.0% w/w of Compound I. In some embodiments, the pharmaceutical composition comprises about 0.55% to about 1.0% w/w of Compound I. In some embodiments, the pharmaceutical composition comprises about 0.6% to about 1.0% w/w of Compound L In some embodiments, the pharmaceutical composition comprises about 0.65% to about 1.0% w/w of Compound I. In some embodiments, the pharmaceutical composition comprises about 0.7% to about 1.0% w/w of Compound I. In some embodiments, the pharmaceutical composition comprises about 0.75% to about 1.0% w/w of Compound I. In some embodiments, the pharmaceutical composition comprises about 0.8% to about 1.0% w/w of Compound I. In some embodiments, the pharmaceutical composition comprises about 0.85% to about 1.0% w/w of Compound I. In some embodiments, the pharmaceutical composition comprises about 0.9% to about 1.0% w/w of Compound I. In some embodiments, the pharmaceutical composition comprises about 0.95% to about 1.0% w/w of Compound I. In some embodiments, the pharmaceutical composition comprises about 0.1% to about 0.9% w/w of Compound I. In some embodiments, the pharmaceutical composition comprises about 0.1% to about 0.8% w/w of Compound I. In some embodiments, the pharmaceutical composition comprises about 0.1% to about 0.7% w/w of Compound I. In some embodiments, the pharmaceutical composition comprises about 0.1% to about 0.6% w/w of Compound I. In some embodiments, the pharmaceutical composition comprises about 0.1% to about 0.5% w/w of Compound I. In some embodiments, the pharmaceutical composition comprises about 0.1% to about 0.4% w/w of Compound I. In some embodiments, the pharmaceutical composition comprises about 0.1% to about 0.3% w/w of Compound L In some embodiments, the pharmaceutical composition comprises about 0.1% to about 0.2% w/w of Compound I. In some embodiments, the pharmaceutical composition comprises about 0.2% to about 0.4% w/w of Compound I. In some embodiments, the pharmaceutical composition comprises about 0.25% to about 0.4% w/w Compound I. In some embodiments, the pharmaceutical composition comprises about 0.3% to about 0.4% w/w Compound I. In some embodiments, the pharmaceutical composition comprises about 0.3% to about 0.35% w/w Compound I. In some embodiments, the pharmaceutical composition comprises about 0.25% to about 0.35% w/w Compound I. In some embodiments, the pharmaceutical composition comprises about 0.25% to about 0.3% w/w Compound I. In some embodiments, the pharmaceutical composition comprises about 0.25% w/w Compound I. In some embodiments, the pharmaceutical composition comprises about 0.3% w/w Compound I. In some embodiments, the pharmaceutical composition comprises about 0.35% w/w Compound I. In some embodiments, the pharmaceutical composition comprises about 0.4% w/w Compound I. In some embodiments, the pharmaceutical composition comprises about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1% of Compound I.

In some embodiments, the pharmaceutical composition comprises 0.05% to 1.0% w/w of Compound I. In some embodiments, the pharmaceutical composition comprises 0.1% to 1.0% w/w of Compound I. In some embodiments, the pharmaceutical composition comprises 0.15% to 1.0% w/w of Compound I. In some embodiments, the pharmaceutical composition comprises 0.2% to 1.0% w/w of Compound I. In some embodiments, the pharmaceutical composition comprises 0.25% to 1.0% w/w of Compound I. In some embodiments, the pharmaceutical composition comprises 0.3% to 1.0% w/w of Compound I. In some embodiments, the pharmaceutical composition comprises 0.35% to 1.0% w/w of Compound I. In some embodiments, the pharmaceutical composition comprises 0.4% to 1.0% w/w of Compound I. In some embodiments, the pharmaceutical composition comprises 0.45% to 1.0% w/w of Compound I. In some embodiments, the pharmaceutical composition comprises 0.5% to 1.0% w/w of Compound I. In some embodiments, the pharmaceutical composition comprises 0.55% to 1.0% w/w of Compound I. In some embodiments, the pharmaceutical composition comprises 0.6% to 1.0% w/w of Compound I. In some embodiments, the pharmaceutical composition comprises 0.65% to 1.0% w/w of Compound I. In some embodiments, the pharmaceutical composition comprises 0.7% to 1.0% w/w of Compound I. In some embodiments, the pharmaceutical composition comprises 0.75% to 1.0% w/w of Compound I. In some embodiments, the pharmaceutical composition comprises 0.8% to 1.0% w/w of Compound I. In some embodiments, the pharmaceutical composition comprises 0.85% to 1.0% w/w of Compound I. In some embodiments, the pharmaceutical composition comprises 0.9% to 1.0% w/w of Compound I. In some embodiments, the pharmaceutical composition comprises 0.95% to 1.0% w/w of Compound I. In some embodiments, the pharmaceutical composition comprises 0.1% to 0.9% w/w of Compound I. In some embodiments, the pharmaceutical composition comprises 0.1% to 0.8% w/w of Compound I. In some embodiments, the pharmaceutical composition comprises 0.1% to 0.7% w/w of Compound I. In some embodiments, the pharmaceutical composition comprises 0.1% to 0.6% w/w of Compound I. In some embodiments, the pharmaceutical composition comprises 0.1% to 0.5% w/w of Compound I. In some embodiments, the pharmaceutical composition comprises 0.1% to 0.4% w/w of Compound I. In some embodiments, the pharmaceutical composition comprises 0.1% to 0.3% w/w of Compound I. In some embodiments, the pharmaceutical composition comprises 0.1% to 0.2% w/w of Compound I. In some embodiments, the pharmaceutical composition comprises 0.2% to 0.4% w/w of Compound I. In some embodiments, the pharmaceutical composition comprises 0.25% to 0.4% w/w Compound I. In some embodiments, the pharmaceutical composition comprises 0.3% to 0.4% w/w Compound I. In some embodiments, the pharmaceutical composition comprises 0.3% to 0.35% w/w Compound I. In some embodiments, the pharmaceutical composition comprises 0.25% to 0.35% w/w Compound I. In some embodiments, the pharmaceutical composition comprises 0.25% to 0.3% w/w Compound I. In some embodiments, the pharmaceutical composition comprises 0.25% w/w Compound I. In some embodiments, the pharmaceutical composition comprises 0.3% w/w Compound I. In some embodiments, the pharmaceutical composition comprises 0.35% w/w Compound I. In some embodiments, the pharmaceutical composition comprises 0.4% w/w Compound I. In some embodiments, the pharmaceutical composition comprises 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1% of Compound I.

In some embodiments, the drug load is equivalent to about 0.05 mg to about 1 mg of Compound I, or pharmaceutically acceptable salt thereof, per 1 gram total weight of the pharmaceutical composition, e.g., about 0.05 mg/g to about 1 mg/g.

In some embodiments, the amount of Compound I or pharmaceutically acceptable salt I thereof in the composition is about 50 μg/g to about 3 mg/g. In some embodiments, the amount of Compound I or pharmaceutically acceptable salt thereof in the composition is about 100 μg/g to about 3 mg/g. In some embodiments, the amount of Compound I or pharmaceutically acceptable salt thereof in the composition is about 150 μg/g to about 3 mg/g. In some embodiments, the amount of Compound I or pharmaceutically acceptable salt thereof in the composition is about 500 μg/g to about 10 mg/g. In some embodiments, the amount of Compound I or pharmaceutically acceptable salt thereof in the composition is about 50 μg/g, about 75 μg/g, about 100 μg/g, about 110 μg/g, about 120 μg/g, about 130 g/g, about 140 μg/g, about 150 μg/g, about 160 μg/g, about 170 μg/g, about 180 μg/g, about 190 μg/g, about 200 μg/g, about 210 μg/g, about 220 μg/g, about 240 μg/g, about 240 μg/g, about 250 μg/g, about 260 μg/g, about 270 μg/g, about 280 μg/g, about 290 μg/g, about 300 g/g, about 310 μg/g, about 320 μg/g, about 330 μg/g, about 340 μg/g, about 350 μg/g, about 360 μg/g, about 370 μg/g, about 380 μg/g, about 390 μg/g, about 400 μg/g, about 410 μg/g, about 420 μg/g, about 430 μg/g, about 440 μg/g, about 450 μg/g, about 460 μg/g, about 470 g/g, about 480 μg/g, about 490 μg/g, about 500 μg/g, about 510 μg/g, about 520 μg/g, about 530 μg/g, about 540 μg/g, about 550 μg/g, about 560 μg/g, about 570 μg/g, about 580 μg/g, about 590 μg/g, about 600 μg/g, about 610 μg/g, about 620 μg/g, about 630 μg/g, about 640 g/g, about 650 μg/g, about 650 μg/g, about 660 μg/g, about 670 μg/g, about 680 μg/g, about 690 μg/g, about 700 μg/g, about 710 μg/g, about 720 μg/g, about 730 μg/g, about 740 μg/g, about 750 μg/g, about 760 μg/g, about 770 μg/g, about 780 μg/g, about 790 μg/g, about 800 g/g, about 810 μg/g, about 820 μg/g, about 830 μg/g, about 840 μg/g, about 850 μg/g, about 860 μg/g, about 870 μg/g, about 880 μg/g, about 890 μg/g, about 900 μg/g, about 910 μg/g about 920 μg/g about 930 μg/g about 940 μg/g about 950 μg/g, about 950 μg/g, about 960 g/g, about 970 μg/g, about 980 μg/g, about 990 μg/g, about 1 mg/g, about 1.1 mg/g, about 1.2 mg/g, about 1.3 mg/g, about 1.4 mg/g, about 1.5 mg/g, 1.6 mg/g, 1.7 mg/g, 1.8 mg/g, 1.9 mg/g, about 2 mg/g, about 2.1 mg/g, about 2.2 mg/g, about 2.3 mg/g, about 2.4 mg/g, about 2.5 mg/g, about 2.6 mg/g, about 2.7 mg/g, about 2.8 mg/g, about 2.9 mg/g, about 3 mg/g, 3.1 mg/g, about 3.2 mg/g, about 3.3 mg/g, about 3.4 mg/g, about 3.5 mg/g, about 3.6 mg/g, about 3.7 mg/g, about 3.8 mg/g, about 3.9 mg/g, about 4 mg/g, about 4.1 mg/g, about 4.2 mg/g, about 4.3 mg/g, about 4.4 mg/g, about 4.5 mg/g, about 4.6 mg/g, about 4.7 mg/g, about 4.8 mg/g, about 4.9 mg/g, about 5 mg/g, about 5.1 mg/g, about 5.2 mg/g, about 5.3 mg/g, about 5.4 mg/g, about 5.5 mg/g, about 5.6 mg/g, about 5.7 mg/g, about 5.8 mg/g, about 5.9 mg/g, about 6 mg/g, about 6.1 mg/g, about 6.2 mg/g, about 6.3 mg/g, about 6.4 mg/g, about 6.5 mg/g, about 6.6 mg/g, about 6.7 mg/g, about 6.8 mg/g, about 6.9 mg/g, about 7 mg/g, about 7.1 mg/g, about 7.2 mg/g, about 7.3 mg/g, about 7.4 mg/g, about 7.5 mg/g, about 7.6 mg/g, about 7.7 mg/g, about 7.8 mg/g, about 7.9 mg/g, about 8 mg/g, about 8.1 mg/g, about 8.2 mg/g, about 8.3 mg/g, about 8.4 mg/g, about 8.5 mg/g, about 8.6 mg/g, about 8.7 mg/g, about 8.8 mg/g, about 8.9 mg/g, about 9 mg/g, about 9.1 mg/g, about 9.2 mg/g, about 9.3 mg/g, about 9.4 mg/g, about 9.5 mg/g, about 9.6 mg/g, about 9.7 mg/g, about 9.8 mg/g, about 9.9 mg/g, or about 10 mg/g.

In some embodiments, the amount of Compound I or pharmaceutically acceptable salt thereof in the composition is 50 μg/g to 3 mg/g. In some embodiments, the amount of Compound I or pharmaceutically acceptable salt thereof in the composition is 100 μg/g to 3 mg/g. In some embodiments, the amount of Compound I or pharmaceutically acceptable salt thereof in the composition is 150 μg/g to 3 mg/g. In some embodiments, the amount of Compound I or pharmaceutically acceptable salt thereof in the composition is 500 μg/g to 10 mg/g. In some embodiments, the amount of Compound I or pharmaceutically acceptable salt thereof in the composition is 50 μg/g, 75 μg/g, 100 μg/g, 110 μg/g, 120 μg/g, 130 μg/g, 140 g/g, 150 μg/g, 160 μg/g, 170 μg/g, 180 μg/g, 190 μg/g, 200 μg/g, 210 μg/g, 220 μg/g, 240 g/g, 240 μg/g, 250 μg/g, 260 μg/g, 270 μg/g, 280 μg/g, 290 μg/g, 300 μg/g, 310 μg/g, 320 g/g, 330 μg/g, 340 μg/g, 350 μg/g, 360 μg/g, 370 μg/g, 380 μg/g, 390 μg/g, 400 μg/g, 410 g/g, 420 μg/g, 430 μg/g, 440 μg/g, 450 μg/g, 460 μg/g, 470 μg/g, 480 μg/g, 490 μg/g, 500 g/g, 510 μg/g, 520 μg/g, 530 μg/g, 540 μg/g, 550 μg/g, 560 μg/g, 570 μg/g, 580 μg/g, 590 g/g, 600 μg/g, 610 μg/g, 620 μg/g, 630 μg/g, 640 μg/g, 650 μg/g, 650 μg/g, 660 μg/g, 670 g/g, 680 μg/g, 690 μg/g, 700 μg/g, 710 μg/g, 720 μg/g, 730 μg/g, 740 μg/g, 750 μg/g, 760 g/g, 770 μg/g, 780 μg/g, 790 μg/g, 800 μg/g, 810 μg/g, 820 μg/g, 830 μg/g, 840 μg/g, 850 g/g, 860 μg/g, 870 μg/g, 880 μg/g, 890 μg/g, 900 μg/g, 910 μg/g 920 μg/g 930 μg/g 940 g/g 950 μg/g, 950 μg/g, 960 μg/g, 970 μg/g, 980 μg/g, 990 μg/g, 1 mg/g, 1.1 mg/g, 1.2 mg/g, 1.3 mg/g, 1.4 mg/g, 1.5 mg/g, 1.6 mg/g, 1.7 mg/g, 1.8 mg/g, 1.9 mg/g, 2 mg/g, 2.1 mg/g, 2.2 mg/g, 2.3 mg/g, 2.4 mg/g, 2.5 mg/g, 2.6 mg/g, 2.7 mg/g, 2.8 mg/g, 2.9 mg/g, 3 mg/g, 3.1 mg/g, 3.2 mg/g, 3.3 mg/g, 3.4 mg/g, 3.5 mg/g, 3.6 mg/g, 3.7 mg/g, 3.8 mg/g, 3.9 mg/g, 4 mg/g, 4.1 mg/g, 4.2 mg/g, 4.3 mg/g, 4.4 mg/g, 4.5 mg/g, 4.6 mg/g, 4.7 mg/g, 4.8 mg/g, 4.9 mg/g, 5 mg/g, 5.1 mg/g, 5.2 mg/g, 5.3 mg/g, 5.4 mg/g, 5.5 mg/g, 5.6 mg/g, 5.7 mg/g, 5.8 mg/g, 5.9 mg/g, 6 mg/g, 6.1 mg/g, 6.2 mg/g, 6.3 mg/g, 6.4 mg/g, 6.5 mg/g, 6.6 mg/g, 6.7 mg/g, 6.8 mg/g, 6.9 mg/g, 7 mg/g, 7.1 mg/g, 7.2 mg/g, 7.3 mg/g, 7.4 mg/g, 7.5 mg/g, 7.6 mg/g, 7.7 mg/g, 7.8 mg/g, 7.9 mg/g, 8 mg/g, 8.1 mg/g, 8.2 mg/g, 8.3 mg/g, 8.4 mg/g, 8.5 mg/g, 8.6 mg/g, 8.7 mg/g, 8.8 mg/g, 8.9 mg/g, 9 mg/g, 9.1 mg/g, 9.2 mg/g, 9.3 mg/g, 9.4 mg/g, 9.5 mg/g, 9.6 mg/g, 9.7 mg/g, 9.8 mg/g, 9.9 mg/g, or 10 mg/g.

In some embodiments, the amount of Compound I in the composition is about 50 g/g to about 3 mg/g. In some embodiments, the amount of Compound I in the composition is about 100 μg/g to about 3 mg/g. In some embodiments, the amount of Compound I in the composition is about 150 μg/g to about 3 mg/g. In some embodiments, the amount of Compound I in the composition is about 500 μg/g to about 10 mg/g. In some embodiments, the amount of Compound I in the composition is about 50 μg/g, about 75 μg/g, about 100 g/g, about 110 μg/g, about 120 μg/g, about 130 μg/g, about 140 μg/g, about 150 μg/g, about 160 μg/g, about 170 μg/g, about 180 μg/g, about 190 μg/g, about 200 μg/g, about 210 μg/g, about 220 μg/g, about 240 μg/g, about 240 μg/g, about 250 μg/g, about 260 μg/g, about 270 g/g, about 280 μg/g, about 290 μg/g, about 300 μg/g, about 310 μg/g, about 320 μg/g, about 330 μg/g, about 340 μg/g, about 350 μg/g, about 360 μg/g, about 370 μg/g, about 380 μg/g, about 390 μg/g, about 400 μg/g, about 410 μg/g, about 420 μg/g, about 430 μg/g, about 440 g/g, about 450 μg/g, about 460 μg/g, about 470 μg/g, about 480 μg/g, about 490 μg/g, about 500 μg/g, about 510 μg/g, about 520 μg/g, about 530 μg/g, about 540 μg/g, about 550 μg/g, about 560 μg/g, about 570 μg/g, about 580 μg/g, about 590 μg/g, about 600 μg/g, about 610 g/g, about 620 μg/g, about 630 μg/g, about 640 μg/g, about 650 μg/g, about 650 μg/g, about 660 μg/g, about 670 μg/g, about 680 μg/g, about 690 μg/g, about 700 μg/g, about 710 μg/g, about 720 μg/g, about 730 μg/g, about 740 μg/g, about 750 μg/g, about 760 μg/g, about 770 g/g, about 780 μg/g, about 790 μg/g, about 800 μg/g, about 810 μg/g, about 820 μg/g, about 830 μg/g, about 840 μg/g, about 850 μg/g, about 860 μg/g, about 870 μg/g, about 880 μg/g, about 890 μg/g, about 900 μg/g, about 910 μg/g about 920 μg/g about 930 μg/g about 940 g/g about 950 μg/g, about 950 μg/g, about 960 μg/g, about 970 μg/g, about 980 μg/g, about 990 μg/g, about 1 mg/g, about 1.1 mg/g, about 1.2 mg/g, about 1.3 mg/g, about 1.4 mg/g, about 1.5 mg/g, 1.6 mg/g, 1.7 mg/g, 1.8 mg/g, 1.9 mg/g, about 2 mg/g, about 2.1 mg/g, about 2.2 mg/g, about 2.3 mg/g, about 2.4 mg/g, about 2.5 mg/g, about 2.6 mg/g, about 2.7 mg/g, about 2.8 mg/g, about 2.9 mg/g, about 3 mg/g, 3.1 mg/g, about 3.2 mg/g, about 3.3 mg/g, about 3.4 mg/g, about 3.5 mg/g, about 3.6 mg/g, about 3.7 mg/g, about 3.8 mg/g, about 3.9 mg/g, about 4 mg/g, about 4.1 mg/g, about 4.2 mg/g, about 4.3 mg/g, about 4.4 mg/g, about 4.5 mg/g, about 4.6 mg/g, about 4.7 mg/g, about 4.8 mg/g, about 4.9 mg/g, about 5 mg/g, about 5.1 mg/g, about 5.2 mg/g, about 5.3 mg/g, about 5.4 mg/g, about 5.5 mg/g, about 5.6 mg/g, about 5.7 mg/g, about 5.8 mg/g, about 5.9 mg/g, about 6 mg/g, about 6.1 mg/g, about 6.2 mg/g, about 6.3 mg/g, about 6.4 mg/g, about 6.5 mg/g, about 6.6 mg/g, about 6.7 mg/g, about 6.8 mg/g, about 6.9 mg/g, about 7 mg/g, about 7.1 mg/g, about 7.2 mg/g, about 7.3 mg/g, about 7.4 mg/g, about 7.5 mg/g, about 7.6 mg/g, about 7.7 mg/g, about 7.8 mg/g, about 7.9 mg/g, about 8 mg/g, about 8.1 mg/g, about 8.2 mg/g, about 8.3 mg/g, about 8.4 mg/g, about 8.5 mg/g, about 8.6 mg/g, about 8.7 mg/g, about 8.8 mg/g, about 8.9 mg/g, about 9 mg/g, about 9.1 mg/g, about 9.2 mg/g, about 9.3 mg/g, about 9.4 mg/g, about 9.5 mg/g, about 9.6 mg/g, about 9.7 mg/g, about 9.8 mg/g, about 9.9 mg/g, or about 10 mg/g.

In some embodiments, the amount of Compound I in the composition is 50 μg/g to 3 mg/g. In some embodiments, the amount of Compound I in the composition is 100 μg/g to 3 mg/g. In some embodiments, the amount of Compound I in the composition is 150 μg/g to 3 mg/g. In some embodiments, the amount of Compound I in the composition is 500 μg/g to 10 mg/g. In some embodiments, the amount of Compound I in the composition is 50 μg/g, 75 μg/g, 100 μg/g, 110 μg/g, 120 μg/g, 130 μg/g, 140 μg/g, 150 μg/g, 160 μg/g, 170 μg/g, 180 g/g, 190 μg/g, 200 μg/g, 210 μg/g, 220 μg/g, 240 μg/g, 240 μg/g, 250 μg/g, 260 μg/g, 270 g/g, 280 μg/g, 290 μg/g, 300 μg/g, 310 μg/g, 320 μg/g, 330 μg/g, 340 μg/g, 350 μg/g, 360 g/g, 370 μg/g, 380 μg/g, 390 μg/g, 400 μg/g, 410 μg/g, 420 μg/g, 430 μg/g, 440 μg/g, 450 g/g, 460 μg/g, 470 μg/g, 480 μg/g, 490 μg/g, 500 μg/g, 510 μg/g, 520 μg/g, 530 μg/g, 540 g/g, 550 μg/g, 560 μg/g, 570 μg/g, 580 μg/g, 590 μg/g, 600 μg/g, 610 μg/g, 620 μg/g, 630 g/g, 640 μg/g, 650 μg/g, 650 μg/g, 660 μg/g, 670 μg/g, 680 μg/g, 690 μg/g, 700 μg/g, 710 g/g, 720 μg/g, 730 μg/g, 740 μg/g, 750 μg/g, 760 μg/g, 770 μg/g, 780 μg/g, 790 μg/g, 800 g/g, 810 μg/g, 820 μg/g, 830 μg/g, 840 μg/g, 850 μg/g, 860 μg/g, 870 μg/g, 880 μg/g, 890 g/g, 900 μg/g, 910 μg/g 920 μg/g 930 μg/g 940 μg/g 950 μg/g, 950 μg/g, 960 μg/g, 970 g/g, 980 μg/g, 990 μg/g, 1 mg/g, 1.1 mg/g, 1.2 mg/g, 1.3 mg/g, 1.4 mg/g, 1.5 mg/g, 1.6 mg/g, 1.7 mg/g, 1.8 mg/g, 1.9 mg/g, 2 mg/g, 2.1 mg/g, 2.2 mg/g, 2.3 mg/g, 2.4 mg/g, 2.5 mg/g, 2.6 mg/g, 2.7 mg/g, 2.8 mg/g, 2.9 mg/g, 3 mg/g, 3.1 mg/g, 3.2 mg/g, 3.3 mg/g, 3.4 mg/g, 3.5 mg/g, 3.6 mg/g, 3.7 mg/g, 3.8 mg/g, 3.9 mg/g, 4 mg/g, 4.1 mg/g, 4.2 mg/g, 4.3 mg/g, 4.4 mg/g, 4.5 mg/g, 4.6 mg/g, 4.7 mg/g, 4.8 mg/g, 4.9 mg/g, 5 mg/g, 5.1 mg/g, 5.2 mg/g, 5.3 mg/g, 5.4 mg/g, 5.5 mg/g, 5.6 mg/g, 5.7 mg/g, 5.8 mg/g, 5.9 mg/g, 6 mg/g, 6.1 mg/g, 6.2 mg/g, 6.3 mg/g, 6.4 mg/g, 6.5 mg/g, 6.6 mg/g, 6.7 mg/g, 6.8 mg/g, 6.9 mg/g, 7 mg/g, 7.1 mg/g, 7.2 mg/g, 7.3 mg/g, 7.4 mg/g, 7.5 mg/g, 7.6 mg/g, 7.7 mg/g, 7.8 mg/g, 7.9 mg/g, 8 mg/g, 8.1 mg/g, 8.2 mg/g, 8.3 mg/g, 8.4 mg/g, 8.5 mg/g, 8.6 mg/g, 8.7 mg/g, 8.8 mg/g, 8.9 mg/g, 9 mg/g, 9.1 mg/g, 9.2 mg/g, 9.3 mg/g, 9.4 mg/g, 9.5 mg/g, 9.6 mg/g, 9.7 mg/g, 9.8 mg/g, 9.9 mg/g, or 10 mg/g.

In some embodiments, the pharmaceutical composition comprises about 50 μg, about 75 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg of Compound I or a pharmaceutically acceptable salt thereof. In some embodiments, the pharmaceutical composition comprises about 1 mg, about 1.2 mg, about 1.5 mg, about 2 mg, about 2.5 mg, or about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of Compound I or a pharmaceutically acceptable salt thereof. In some embodiments, the pharmaceutical composition comprises about 150 μg of Compound I or a pharmaceutically acceptable salt thereof. In some embodiments, the pharmaceutical composition comprises about 300 μg of Compound I or a pharmaceutically acceptable salt thereof. In some embodiments, the pharmaceutical composition comprises about 500 μg of Compound I or a pharmaceutically acceptable salt thereof. In some embodiments, the pharmaceutical composition comprises about 600 μg of Compound I or a pharmaceutically acceptable salt thereof. In some embodiments, the pharmaceutical composition comprises about 900 μg of Compound I or a pharmaceutically acceptable salt thereof. In some embodiments, the pharmaceutical composition comprises about 1.2 mg of Compound I or a pharmaceutically acceptable salt thereof. In some embodiments, the pharmaceutical composition comprises about 3 mg of Compound I or a pharmaceutically acceptable salt thereof. In some embodiments, the pharmaceutical composition comprises about 10 mg of Compound I or a pharmaceutically acceptable salt thereof.

In some embodiments, the pharmaceutical composition comprises 50 μg, 75 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1000 μg of Compound I or a pharmaceutically acceptable salt thereof. In some embodiments, the pharmaceutical composition comprises 1 mg, 1.2 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg of Compound I or a pharmaceutically acceptable salt thereof. In some embodiments, the pharmaceutical composition comprises 150 μg of Compound I or a pharmaceutically acceptable salt thereof. In some embodiments, the pharmaceutical composition comprises 300 μg of Compound I or a pharmaceutically acceptable salt thereof. In some embodiments, the pharmaceutical composition comprises 500 μg of Compound I or a pharmaceutically acceptable salt thereof. In some embodiments, the pharmaceutical composition comprises 600 μg of Compound I or a pharmaceutically acceptable salt thereof. In some embodiments, the pharmaceutical composition comprises 900 μg of Compound I or a pharmaceutically acceptable salt thereof. In some embodiments, the pharmaceutical composition comprises 1.2 mg of Compound I or a pharmaceutically acceptable salt thereof. In some embodiments, the pharmaceutical composition comprises 3 mg of Compound I or a pharmaceutically acceptable salt thereof. In some embodiments, the pharmaceutical composition comprises 10 mg of Compound I or a pharmaceutically acceptable salt thereof.

In some embodiments, the pharmaceutical composition comprises about 50 μg, about 75 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg of Compound I. In some embodiments, the pharmaceutical composition comprises about 1 mg, about 1.2 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of Compound I. In some embodiments, the pharmaceutical composition comprises about 150 μg of Compound I. In some embodiments, the pharmaceutical composition comprises about 300 μg of Compound I. In some embodiments, the pharmaceutical composition comprises about 500 μg of Compound I. In some embodiments, the pharmaceutical composition comprises about 600 μg of Compound I. In some embodiments, the pharmaceutical composition comprises about 900 μg of Compound I. In some embodiments, the pharmaceutical composition comprises about 1.2 mg of Compound I. In some embodiments, the pharmaceutical composition comprises about 3 mg of Compound I. In some embodiments, the pharmaceutical composition comprises about 10 mg of Compound I.

In some embodiments, the pharmaceutical composition comprises 50 μg, 75 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1000 μg of Compound I. In some embodiments, the pharmaceutical composition comprises 1 mg, 1.2 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg of Compound I. In some embodiments, the pharmaceutical composition comprises 150 μg of Compound I. In some embodiments, the pharmaceutical composition comprises 300 μg of Compound I. In some embodiments, the pharmaceutical composition comprises 500 μg of Compound I. In some embodiments, the pharmaceutical composition comprises 600 μg of Compound I. In some embodiments, the pharmaceutical composition comprises 900 μg of Compound I. In some embodiments, the pharmaceutical composition comprises 1.2 mg of Compound I. In some embodiments, the pharmaceutical composition comprises 3 mg of Compound I. In some embodiments, the pharmaceutical composition comprises 10 mg of Compound I.

In some embodiments, the amount of Compound I or pharmaceutically acceptable salt thereof in the composition is about 2 mg/g to about 4 mg/g. In some embodiments, the amount of Compound I or pharmaceutically acceptable salt thereof in the composition is about 2 mg/g to about 3 mg/g. In some embodiments, the amount of Compound I or pharmaceutically acceptable salt thereof in the composition is about 2 mg/g, about 2.1 mg/g, about 2.2 mg/g, about 2.3 mg/g, about 2.4 mg/g, about 2.5 mg/g, about 2.6 mg/g, about 2.7 mg/g, about 2.8 mg/g, about 2.9 mg/g, about 3 mg/g, about 3.1 mg/g, about 3.2 mg/g, about 3.3 mg/g, about 3.4 mg/g, about 3.5 mg/g, about 3.6 mg/g, about 3.7 mg/g, about 3.8 mg/g, about 3.9 mg/g, or about 4 mg/g.

In some embodiments, the amount of Compound I or pharmaceutically acceptable salt thereof in the composition is 2 mg/g to 4 mg/g. In some embodiments, the amount of Compound I or pharmaceutically acceptable salt thereof in the composition is 2 mg/g to 3 mg/g. In some embodiments, the amount of Compound I or pharmaceutically acceptable salt thereof in the composition is 2 mg/g, 2.1 mg/g, 2.2 mg/g, 2.3 mg/g, 2.4 mg/g, 2.5 mg/g, 2.6 mg/g, 2.7 mg/g, 2.8 mg/g, 2.9 mg/g, 3 mg/g, 3.1 mg/g, 3.2 mg/g, 3.3 mg/g, 3.4 mg/g, 3.5 mg/g, 3.6 mg/g, 3.7 mg/g, 3.8 mg/g, 3.9 mg/g, or 4 mg/g.

In some embodiments, the amount of Compound I in the composition is about 2 mg/g to about 4 mg/g. In some embodiments, the amount of Compound I in the composition is about 2 mg/g to about 3 mg/g. In some embodiments, the amount of Compound I in the composition is about 2 mg/g, about 2.1 mg/g, about 2.2 mg/g, about 2.3 mg/g, about 2.4 mg/g, about 2.5 mg/g, about 2.6 mg/g, about 2.7 mg/g, about 2.8 mg/g, about 2.9 mg/g, about 3 mg/g, about 3.1 mg/g, about 3.2 mg/g, about 3.3 mg/g, about 3.4 mg/g, about 3.5 mg/g, about 3.6 mg/g, about 3.7 mg/g, about 3.8 mg/g, about 3.9 mg/g, or about 4 mg/g.

In some embodiments, the amount of Compound I in the composition is 2 mg/g to 4 mg/g. In some embodiments, the amount of Compound I in the composition is 2 mg/g to 3 mg/g. In some embodiments, the amount of Compound I thereof in the composition is 2 mg/g, 2.1 mg/g, 2.2 mg/g, 2.3 mg/g, 2.4 mg/g, 2.5 mg/g, 2.6 mg/g, 2.7 mg/g, 2.8 mg/g, 2.9 mg/g, 3 mg/g, 3.1 mg/g, 3.2 mg/g, 3.3 mg/g, 3.4 mg/g, 3.5 mg/g, 3.6 mg/g, 3.7 mg/g, 3.8 mg/g, 3.9 mg/g, or 4 mg/g.

In some embodiments, the pharmaceutical composition comprises about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg of Compound I or a pharmaceutically acceptable salt thereof. In some embodiments, the pharmaceutical composition comprises about 1 mg, about 1.5 mg, about 2 mg, about 2.5 mg, or about 3 mg of Compound I or a pharmaceutically acceptable salt thereof. In some embodiments, the pharmaceutical composition comprises about 150 μg of Compound I or a pharmaceutically acceptable salt thereof. In some embodiments, the pharmaceutical composition comprises about 3 mg of Compound I or a pharmaceutically acceptable salt thereof.

In some embodiments, the pharmaceutical composition comprises 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1000 μg of Compound I or a pharmaceutically acceptable salt thereof. In some embodiments, the pharmaceutical composition comprises 1 mg, 1.5 mg, 2 mg, 2.5 mg, or 3 mg of Compound I or a pharmaceutically acceptable salt thereof. In some embodiments, the pharmaceutical composition comprises 150 μg of Compound I or a pharmaceutically acceptable salt thereof. In some embodiments, the pharmaceutical composition comprises 3 mg of Compound I or a pharmaceutically acceptable salt thereof.

In some embodiments, the pharmaceutical composition comprises about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg of Compound I. In some embodiments, the pharmaceutical composition comprises about 1 mg, about 1.5 mg, about 2 mg, about 2.5 mg, or about 3 mg of Compound I. In some embodiments, the pharmaceutical composition comprises about 150 g of Compound I. In some embodiments, the pharmaceutical composition comprises about 3 mg of Compound I.

In some embodiments, the pharmaceutical composition comprises 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1000 μg of Compound I. In some embodiments, the pharmaceutical composition comprises 1 mg, 1.5 mg, 2 mg, 2.5 mg, or 3 mg of Compound I. In some embodiments, the pharmaceutical composition comprises 150 μg of Compound I. In some embodiments, the pharmaceutical composition comprises 3 mg of Compound I.

In some embodiments, the amount of monoglycerides in said glycerides is greater than about 30% w/w. In some embodiments, the amount of monoglycerides in said glycerides is from about 30% to about 100% w/w. In some embodiments, the amount of monoglycerides in said glycerides is from about 30% to about 90% w/w. In some embodiments, the amount of monoglycerides in said glycerides is from about 30% to about 80% w/w. In some embodiments, the amount of monoglycerides in said glycerides is from about 30% to about 70% w/w. In some embodiments, the amount of monoglycerides in said glycerides is from about 30% to about 60% w/w. In some embodiments, the amount of monoglycerides in said glycerides is from about 30% to about 50% w/w. In some embodiments, the amount of monoglycerides in said glycerides is from about 30% to about 40% w/w. In some embodiments, the amount of monoglycerides in said glycerides is from about 40% to about 100% w/w. In some embodiments, the amount of monoglycerides in said glycerides is from about 40% to about 90% w/w. In some embodiments, the amount of monoglycerides in said glycerides is from about 40% to about 80% w/w. In some embodiments, the amount of monoglycerides in said glycerides is from about 40% to about 70% w/w. In some embodiments, the amount of monoglycerides in said glycerides is from about 40% to about 60% w/w. In some embodiments, the amount of monoglycerides in said glycerides is from about 40% to about 50% w/w. In some embodiments, the amount of monoglycerides in said glycerides is from about 50% to about 100% w/w. In some embodiments, the amount of monoglycerides in said glycerides is from about 50% to about 90% w/w. In some embodiments, the amount of monoglycerides in said glycerides is from about 50% to about 80% w/w. In some embodiments, the amount of monoglycerides in said glycerides is from about 50% to about 70% w/w. In some embodiments, the amount of monoglycerides in said glycerides is from about 50% to about 60% w/w. In some embodiments, the amount of monoglycerides in said glycerides is from about 60% to about 100% w/w. In some embodiments, the amount of monoglycerides in said glycerides is from about 60% to about 90% w/w. In some embodiments, the amount of monoglycerides in said glycerides is from about 60% to about 80% w/w. In some embodiments, the amount of monoglycerides in said glycerides is from about 60% to about 70% w/w. In some embodiments, the amount of monoglycerides in said glycerides is from about 70% to about 100% w/w. In some embodiments, the amount of monoglycerides in said glycerides is from about 70% to about 90% w/w. In some embodiments, the amount of monoglycerides in said glycerides is from about 70% to about 80% w/w. In some embodiments, the amount of monoglycerides in said glycerides is from about 80% to about 100% w/w. In some embodiments, the amount of monoglycerides in said glycerides is from about 80% to about 90% w/w. In some embodiments, the amount of monoglycerides in said glycerides is from about 90% to about 100% w/w. In some embodiments, the amount of monoglycerides in said glycerides is about 30%, about 35%, about 40%, about 45%, about 50%, about 55%, about 60%, about 65%, about 70%, about 75%, about 80%, about 85%, about 90% w/w, about 95% w/w, or about 100% w/w.

In some embodiments, the amount of monoglycerides in said glycerides is greater than 30% w/w. In some embodiments, the amount of monoglycerides in said glycerides is from 30% to 100% w/w. In some embodiments, the amount of monoglycerides in said glycerides is from 30% to 90% w/w. In some embodiments, the amount of monoglycerides in said glycerides is from 30% to 80% w/w. In some embodiments, the amount of monoglycerides in said glycerides is from 30% to 70% w/w. In some embodiments, the amount of monoglycerides in said glycerides is from 30% to 60% w/w. In some embodiments, the amount of monoglycerides in said glycerides is from 30% to 50% w/w. In some embodiments, the amount of monoglycerides in said glycerides is from 30% to 40% w/w. In some embodiments, the amount of monoglycerides in said glycerides is from 40% to 100% w/w. In some embodiments, the amount of monoglycerides in said glycerides is from 40% to 90% w/w. In some embodiments, the amount of monoglycerides in said glycerides is from 40% to 80% w/w. In some embodiments, the amount of monoglycerides in said glycerides is from 40% to 70% w/w. In some embodiments, the amount of monoglycerides in said glycerides is from 40% to 60% w/w. In some embodiments, the amount of monoglycerides in said glycerides is from 40% to 50% w/w. In some embodiments, the amount of monoglycerides in said glycerides is from 50% to 100% w/w. In some embodiments, the amount of monoglycerides in said glycerides is from 50% to 90% w/w. In some embodiments, the amount of monoglycerides in said glycerides is from 50% to 80% w/w. In some embodiments, the amount of monoglycerides in said glycerides is from 50% to 70% w/w. In some embodiments, the amount of monoglycerides in said glycerides is from 50% to 60% w/w. In some embodiments, the amount of monoglycerides in said glycerides is from 60% to 100% w/w. In some embodiments, the amount of monoglycerides in said glycerides is from 60% to 90% w/w. In some embodiments, the amount of monoglycerides in said glycerides is from 60% to 80% w/w. In some embodiments, the amount of monoglycerides in said glycerides is from 60% to 70% w/w. In some embodiments, the amount of monoglycerides in said glycerides is from 70% to 100% w/w. In some embodiments, the amount of monoglycerides in said glycerides is from 70% to 90% w/w. In some embodiments, the amount of monoglycerides in said glycerides is from 70% to 80% w/w. In some embodiments, the amount of monoglycerides in said glycerides is from 80% to 100% w/w. In some embodiments, the amount of monoglycerides in said glycerides is from 80% to 90% w/w. In some embodiments, the amount of monoglycerides in said glycerides is from 90% to 100% w/w. In some embodiments, the amount of monoglycerides in said glycerides is 30%, 35%, 40%, 45%, 50%, 55%, 60%, 65%, 70%, 75%, 80%, 85%, 90% w/w, 95% w/w, or 100% w/w.

In some embodiments, the one or more glycerides comprises one or more mono-, di-, or tri-glycerides or a combination thereof. Exemplary glycerides include but are not limited to glyceryl mono-/di-caprylocaprate, glyceryl mono-/di-caprylocaprate, glyceryl monocaprylate Type 1, glyceryl monocaprylate Type 1, glyceryl monocaprylocaprate Type 1, glyceryl monolinoleate, equivalents thereof and combinations thereof. In some embodiments, the one or more glycerides comprises glyceryl monocaprylate. In some embodiments, the one or more glycerides comprises glyceryl monolinoleate. In some embodiments, the one or more glycerides comprises glyceryl monocaprylate and glyceryl monolinoleate.

In some embodiments, the pharmaceutical composition comprises one or more monoesters, diesters or triesters of fatty acids attached to a glycerol moiety (glycerides) of different chain lengths alone or in combination with mono- or diesters of fatty acids attached to propylene glycol moiety. In some embodiments, the pharmaceutical composition comprises one or more monoesters, diesters or triesters of fatty acids attached to a glycerol moiety (glycerides) of different chain lengths. In some embodiments, the pharmaceutical composition comprises one or more monoesters, diesters or triesters of fatty acids attached to a glycerol moiety (glycerides) of different chain lengths in combination with mono- or di-esters of fatty acids attached to propylene glycol moiety. In some embodiments, the pharmaceutical composition comprises one or more monoesters of fatty acids attached to a glycerol moiety (glycerides) of different chain lengths. In some embodiments, the pharmaceutical composition comprises one or more diesters of fatty acids attached to a glycerol moiety (glycerides) of different chain lengths. In some embodiments, the pharmaceutical composition comprises one or more triesters of fatty acids attached to a glycerol moiety (glycerides) of different chain lengths. In some embodiments, the pharmaceutical composition comprises one or more monoesters of fatty acids attached to propylene glycol moiety. In some embodiments, the pharmaceutical composition comprises one or more diesters of fatty acids attached to propylene glycol moiety.

Exemplary monoesters and diesters of fatty acids attached to a glycerol moiety include, but are not limited to, glyceryl mono-/di-caprylocaprate, glyceryl monocaprylate Type 1, glyceryl monocaprylate Type 1, glyceryl monocaprylocaprate Type 1, glyceryl monolinoleate or equivalents thereof. Exemplary monoesters and diesters of fatty acids attached to a propylene glycol moiety (propylene glycol esters) include, but are not limited to, propylene glycol mono caprylate Type II, propylene glycol monolaurate, propylene glycol dicaprylate/dicaprate.

In some embodiments, the pharmaceutical composition comprises one or more glycerides of different fatty acids chain length distribution. In some embodiments, the pharmaceutical composition comprises one or more medium chain mono-, di- or triglycerides (e.g., Caproic (C6), Caprylic (C8), Capric (C10), lauric (C12)). In some embodiments, the pharmaceutical composition comprises one or more long chain triglycerides (e.g., myristic (C14), Palmitic (C16), Stearic (C18), Oleic (C18), Linoleic (C18)). In some embodiments, the pharmaceutical composition comprises a combination of medium chain mono-, di- or triglycerides and long chain triglycerides.

In some embodiments, the one or more glycerides in the pharmaceutical composition is selected from the group consisting of glyceryl monocaprylate, glyceryl monolinoleate and a combination thereof. In some embodiments, the glyceride is glyceryl monocaprylate. In some embodiments, the glyceride is glyceryl monolinoleate. In some embodiments, the pharmaceutical composition comprises glyceryl monocaprylate and glyceryl monolinoleate.

In some embodiments, glyceryl monocaprylate is in an amount from about 20% to about 80% w/w. In some embodiments, glyceryl monocaprylate is in an amount from about 20% to about 70% w/w. In some embodiments, glyceryl monocaprylate is in an amount from about 20% to about 60% w/w. In some embodiments, glyceryl monocaprylate is in an amount from about 20% to about 50% w/w. In some embodiments, glyceryl monocaprylate is in an amount from about 20% to about 40% w/w. In some embodiments, glyceryl monocaprylate is in an amount from about 20% to about 30% w/w. In some embodiments, glyceryl monocaprylate is in an amount from about 30% to about 80% w/w. In some embodiments, glyceryl monocaprylate is in an amount from about 30% to about 70% w/w. In some embodiments, glyceryl monocaprylate is in an amount from about 30% to about 60% w/w. In some embodiments, glyceryl monocaprylate is in an amount from about 30% to about 50% w/w. In some embodiments, glyceryl monocaprylate is in an amount from about 30% to about 40% w/w. In some embodiments, glyceryl monocaprylate is in an amount from about 40% to about 80% w/w. In some embodiments, glyceryl monocaprylate is in an amount from about 40% to about 70% w/w. In some embodiments, glyceryl monocaprylate is in an amount from about 40% to about 60% w/w. In some embodiments, glyceryl monocaprylate is in an amount from about 40% to about 50% w/w. In some embodiments, glyceryl monocaprylate is in an amount from about 50% to about 80% w/w. In some embodiments, glyceryl monocaprylate is in an amount from about 50% to about 70% w/w. In some embodiments, glyceryl monocaprylate is in an amount from about 50% to about 60% w/w. In some embodiments, glyceryl monocaprylate is in an amount from about 60% to about 80% w/w. In some embodiments, glyceryl monocaprylate is in an amount from about 60% to about 70% w/w. In some embodiments, glyceryl monocaprylate is in an amount from about 70% to about 80% w/w. In some embodiments, glyceryl monocaprylate is in an amount of about 20%, about 30%, about 40%, about 45%, about 50%, about 55%, about 60%, about 65%, about 70%, about 75%, about 80% w/w. In some embodiments, glyceryl monocaprylate is in an amount of about 46%, about 47%, about 48%, about 49%, about 50%, about 51%, about 52%, about 53%, about 54%, about 56%, about 57%, about 58%, about 59%, about 61%, about 62%, about 63%, about 64% w/w. In some embodiments, glyceryl monocaprylate is in an amount of about 49% w/w. In some embodiments, glyceryl monocaprylate is in an amount of about 50% w/w. In some embodiments, glyceryl monocaprylate is in an amount of about 51% w/w. In some embodiments, glyceryl monocaprylate is in an amount of about 49.5%, about 49.6%, about 49.7%, about 49.8%, about 49.9%, about 50.0%, about 50.1%, about 50.2%, about 50.3%, about 50.4%, about 50.5% w/w. In some embodiments, glyceryl monocaprylate is in an amount of about 49.8% w/w. In some embodiments, glyceryl monocaprylate is in an amount of about 59% w/w. In some embodiments, glyceryl monocaprylate is in an amount of about 60%. In some embodiments, glyceryl monocaprylate is in an amount of about 61% w/w. In some embodiments, glyceryl monocaprylate is in an amount of about 59.5%, about 59.6%, 59.7%, about 59.8%, about 59.9%, about 6.0%, about 6.1%, about 6.2%, about 6.3% w/w. In some embodiments, glyceryl monocaprylate is in an amount of about 59.8% w/w.

In some embodiments, glyceryl monocaprylate is in an amount from 20% to 80% w/w. In some embodiments, glyceryl monocaprylate is in an amount from 20% to 70% w/w. In some embodiments, glyceryl monocaprylate is in an amount from 20% to 60% w/w. In some embodiments, glyceryl monocaprylate is in an amount from 20% to 50% w/w. In some embodiments, glyceryl monocaprylate is in an amount from 20% to 40% w/w. In some embodiments, glyceryl monocaprylate is in an amount from 20% to 30% w/w. In some embodiments, glyceryl monocaprylate is in an amount from 30% to 80% w/w. In some embodiments, glyceryl monocaprylate is in an amount from 30% to 70% w/w. In some embodiments, glyceryl monocaprylate is in an amount from 30% to 60% w/w. In some embodiments, glyceryl monocaprylate is in an amount from 30% to 50% w/w. In some embodiments, glyceryl monocaprylate is in an amount from 30% to 40% w/w. In some embodiments, glyceryl monocaprylate is in an amount from 40% to 80% w/w. In some embodiments, glyceryl monocaprylate is in an amount from 40% to 70% w/w. In some embodiments, glyceryl monocaprylate is in an amount from 40% to 60% w/w. In some embodiments, glyceryl monocaprylate is in an amount from 40% to 50% w/w. In some embodiments, glyceryl monocaprylate is in an amount from 50% to 80% w/w. In some embodiments, glyceryl monocaprylate is in an amount from 50% to 70% w/w. In some embodiments, glyceryl monocaprylate is in an amount from 50% to 60% w/w. In some embodiments, glyceryl monocaprylate is in an amount from 60% to 80% w/w. In some embodiments, glyceryl monocaprylate is in an amount from 60% to 70% w/w. In some embodiments, glyceryl monocaprylate is in an amount from 70% to 80% w/w. In some embodiments, glyceryl monocaprylate is in an amount of 20%, 30%, 40%, 45%, 50%, 55%, 60%, 65%, 70%, 75%, 80% w/w. In some embodiments, glyceryl monocaprylate is in an amount of 46%, 47%, 48%, 49%, 50%, 51%, 52%, 53%, 54%, 56%, 57%, 58%, 59%, 61%, 62%, 63%, 64% w/w. In some embodiments, glyceryl monocaprylate is in an amount of 49% w/w. In some embodiments, glyceryl monocaprylate is in an amount of 50% w/w. In some embodiment, glyceryl monocaprylate is in an amount of 51% w/w. In some embodiments, glyceryl monocaprylate is in an amount of 49.5%, 49.6%, 49.7%, 49.8%, 49.9%, 50.0%, 50.1%, 50.2%, 50.3%, 50.4%, 50.5% w/w. In some embodiments, glyceryl monocaprylate is in an amount of 49.8% w/w. In some embodiments, glyceryl monocaprylate is in an amount of 59% w/w. In some embodiments, glyceryl monocaprylate is in an amount of 60%. In some embodiments, glyceryl monocaprylate is in an amount of 61% w/w. In some embodiments, glyceryl monocaprylate is in an amount of 59.5%, 59.6%, 59.7%, 59.8%, 59.9%, 6.0%, 6.1%, 6.2%, 6.3% w/w. In some embodiments, glyceryl monocaprylate is in an amount of 59.8% w/w.

In some embodiments, the glyceryl monolinoleate is in an amount from about 10% to about 30% w/w. In some embodiments, glyceryl monolinoleate is in an amount from about 15% to about 30% w/w. In some embodiments, glyceryl monolinoleate is in an amount from about 15% to about 25% w/w. In some embodiments, glyceryl monolinoleate is in an amount from about 15% to about 20% w/w. In some embodiments, glyceryl monolinoleate is in an amount from about 20% to about 30% w/w. In some embodiments, glyceryl monolinoleate is in an amount from about 25% to about 30% w/w. In some embodiments, glyceryl monolinoleate is in an amount of about 10%, 15%, 20%, 25%, 30% w/w. In some embodiments, glyceryl monolinoleate is in an amount from about 15%, 16%, 17%, 18%, 19%, 20%, 21%, 22%, 23%, 24%, 25% w/w. In some embodiments, glyceryl monolinoleate is in an amount of about 19%. In some embodiments, glyceryl monolinoleate is in an amount of about 20% w/w. In some embodiments, glyceryl monolinoleate is in an amount of about 21% w/w. In some embodiments, glyceryl monolinoleate is in an amount from about 19.5%, about 19.6%, about 19.7%, about 19.8%, about 19.9%, about 20.1%, about 20.2%, about 20.3%, about 2.4%, about 2.5% w/w. In some embodiments, glyceryl monolinoleate is in an amount of about 19.8% w/w. In some embodiments, glyceryl monolinoleate is in an amount of about 19.9% w/w. In some embodiments, glyceryl monolinoleate is in an amount of about 20% w/w. In some embodiments, glyceryl monolinoleate is in an amount from about 22.0%, about 22.1%, about 22.2%, about 22.3%, about 22.4%, about 22.5%, about 22.6%, about 22.7%, about 22.8%, about 22.9% w/w. In some embodiments, glyceryl monolinoleate is in an amount of about 22.3% w/w. In some embodiments, glyceryl monolinoleate is in an amount of about 22.4% w/w. In some embodiments, glyceryl monolinoleate is in an amount of about 22.5% w/w.

In some embodiments, the glyceryl monolinoleate is in an amount from 10% to 30% w/w. In some embodiments, glyceryl monolinoleate is in an amount from 15% to 30% w/w. In some embodiments, glyceryl monolinoleate is in an amount from 15% to 25% w/w. In some embodiments, glyceryl monolinoleate is in an amount from 15% to 20% w/w. In some embodiments, glyceryl monolinoleate is in an amount from 20% to 30% w/w. In some embodiments, glyceryl monolinoleate is in an amount from 25% to 30% w/w. In some embodiments, glyceryl monolinoleate is in an amount of 10%, 15%, 20%, 25%, 30% w/w. In some embodiments, glyceryl monolinoleate is in an amount from 15%, 16%, 17%, 18%, 19%, 20%, 21%, 22%, 23%, 24%, 25% w/w. In some embodiments, glyceryl monolinoleate is in an amount of 19%. In some embodiments, glyceryl monolinoleate is in an amount of 20% w/w. In some embodiments, glyceryl monolinoleate is in an amount of 21% w/w. In some embodiments, glyceryl monolinoleate is in an amount from 19.5%, 19.6%, 19.7%, 19.8%, 19.9%, 20.1%, 20.2%, 20.3%, 20.4%, 20.5% w/w. In some embodiments, glyceryl monolinoleate is in an amount of 19.8% w/w. In some embodiments, glyceryl monolinoleate is in an amount of 19.9% w/w. In some embodiments, glyceryl monolinoleate is in an amount of 20% w/w. In some embodiments, glyceryl monolinoleate is in an amount from 22.0%, 22.1%, 22.2%, 22.3%, 22.4%, 22.5%, 22.6%, 22.7%, 22.8%, 22.9% w/w. In some embodiments, glyceryl monolinoleate is in an amount of 22.3% w/w. In some embodiments, glyceryl monolinoleate is in an amount of 22.4% w/w. In some embodiments, glyceryl monolinoleate is in an amount of 22.5% w/w.

In some embodiments, the pharmaceutical composition further comprises a surfactant. In some embodiments, the surfactant is stearoyl polyoxyl-32 glycerides. In some embodiments, the surfactant is lauroyl polyoxyl-32 glycerides.

In some embodiments, the stearoyl polyoxyl-32 glycerides is in an amount from about 5.0% to about 20% w/w. In some embodiments, the stearoyl polyoxyl-32 glycerides is in an amount from about 5.0% to about 15% w/w. In some embodiments, the stearoyl polyoxyl-32 glycerides is in an amount from about 5.0% to about 10% w/w. In some embodiments, stearoyl polyoxyl-32 glycerides is in an amount from about 5.0% to about 9.0% w/w. In some embodiments, stearoyl polyoxyl-32 glycerides is in an amount from about 5.0% to about 8.0% w/w. In some embodiments, stearoyl polyoxyl-32 glycerides is in an amount from about 5.0% to about 7.5% w/w. In some embodiments, stearoyl polyoxyl-32 glycerides is in an amount from about 6.0% to about 10% w/w. In some embodiments, stearoyl polyoxyl-32 glycerides is in an amount from about 6.0% to about 9.0% w/w. In some embodiments, stearoyl polyoxyl-32 glycerides is in an amount from about 6.0% to about 8.0% w/w. In some embodiments, stearoyl polyoxyl-32 glycerides is in an amount from about 6.0% to about 7.5% w/w. In some embodiments, stearoyl polyoxyl-32 glycerides is in an amount from about 7.0% to about 10.0% w/w. In some embodiments, stearoyl polyoxyl-32 glycerides is in an amount from about 7.0% to about 9.0% w/w. In some embodiments, stearoyl polyoxyl-32 glycerides is in an amount from about 7.0% to about 8.0% w/w. In some embodiments, stearoyl polyoxyl-32 glycerides is in an amount from about 7.0% to about 7.5% w/w. In some embodiments, the stearoyl polyoxyl-32 glycerides is in an amount of about 7.0%, about 7.1%, about 7.2%, about 7.3%, about 7.4%, about 7.5%, about 7.6%, about 7.7%, about 7.8%, about 7.9%, about 8.0% w/w. In some embodiments, the stearoyl polyoxyl-32 glycerides is in an amount of about 7.4% w/w. In some embodiments, the stearoyl polyoxyl-32 glycerides is in an amount of about 7.5% w/w. In some embodiments, the stearoyl polyoxyl-32 glycerides is in an amount of about 10% w/w. In some embodiments, the stearoyl polyoxyl-32 glycerides is in an amount of about 15% w/w. In some embodiments, the stearoyl polyoxyl-32 glycerides is in an amount of about 20% w/w.

In some embodiments, the stearoyl polyoxyl-32 glycerides is in an amount from 5.0% to 20% w/w. In some embodiments, the stearoyl polyoxyl-32 glycerides is in an amount from 5.0% to 15% w/w. In some embodiments, the stearoyl polyoxyl-32 glycerides is in an amount from 5.0% to 10% w/w. In some embodiments, stearoyl polyoxyl-32 glycerides is in an amount from 5.0% to 9.0% w/w. In some embodiments, stearoyl polyoxyl-32 glycerides is in an amount from 5.0% to 8.0% w/w. In some embodiments, stearoyl polyoxyl-32 glycerides is in an amount from 5.0% to 7.5% w/w. In some embodiments, stearoyl polyoxyl-32 glycerides is in an amount from 6.0% to 10% w/w. In some embodiments, stearoyl polyoxyl-32 glycerides is in an amount from 6.0% to 9.0% w/w. In some embodiments, stearoyl polyoxyl-32 glycerides is in an amount from 6.0% to 8.0% w/w. In some embodiments, stearoyl polyoxyl-32 glycerides is in an amount from 6.0% to 7.5% w/w. In some embodiments, stearoyl polyoxyl-32 glycerides is in an amount from 7.0% to 10.0% w/w. In some embodiments, stearoyl polyoxyl-32 glycerides is in an amount from 7.0% to 9.0% w/w. In some embodiments, stearoyl polyoxyl-32 glycerides is in an amount from 7.0% to 8.0% w/w. In some embodiments, stearoyl polyoxyl-32 glycerides is in an amount from 7.0% to 7.5% w/w. In some embodiments, the stearoyl polyoxyl-32 glycerides is in an amount of 7.0%, 7.1%, 7.2%, 7.3%, 7.4%, 7.5%, 7.6%, 7.7%, 7.8%, 7.9%, 8.0% w/w. In some embodiments, the stearoyl polyoxyl-32 glycerides is in an amount of 7.4% w/w. In some embodiments, the stearoyl polyoxyl-32 glycerides is in an amount of 7.5% w/w. In some embodiments, the stearoyl polyoxyl-32 glycerides is in an amount of 10% w/w. In some embodiments, the stearoyl polyoxyl-32 glycerides is in an amount of 15% w/w. In some embodiments, the stearoyl polyoxyl-32 glycerides is in an amount of 20% w/w.

In some embodiments, the lauroyl polyoxyl-32 glycerides is in an amount from about 5.0% to about 20% w/w. In some embodiments, the lauroyl polyoxyl-32 glycerides is in an amount from about 5.0% to about 15% w/w. In some embodiments, the lauroyl polyoxyl-32 glycerides is in an amount from about 5.0% to about 10% w/w. In some embodiments, the lauroyl polyoxyl-32 glycerides is in an amount from about 5.0% to about 9.0% w/w. In some embodiments, the lauroyl polyoxyl-32 glycerides is in an amount from about 5.0% to about 8.0% w/w. In some embodiments, the lauroyl polyoxyl-32 glycerides is in an amount from about 5.0% to about 7.5% w/w. In some embodiments, the lauroyl polyoxyl-32 glycerides is in an amount from about 6.0% to about 10% w/w. In some embodiments, the lauroyl polyoxyl-32 glycerides is in an amount from about 6.0% to about 9.0% w/w. In some embodiments, the lauroyl polyoxyl-32 glycerides is in an amount from about 6.0% to about 8.0% w/w. In some embodiments, the lauroyl polyoxyl-32 glycerides is in an amount from about 6.0% to about 7.5% w/w. In some embodiments, the lauroyl polyoxyl-32 glycerides is in an amount from about 7.0% to about 10.0% w/w. In some embodiments, the lauroyl polyoxyl-32 glycerides is in an amount from about 7.0% to about 9.0% w/w. In some embodiments, the lauroyl polyoxyl-32 glycerides is in an amount from about 7.0% to about 8.0% w/w. In some embodiments, the lauroyl polyoxyl-32 glycerides is in an amount from about 7.0% to about 7.5% w/w. In some embodiments, the lauroyl polyoxyl-32 glycerides is in an amount of about 7.0%, about 7.1%, about 7.2%, about 7.3%, about 7.4%, about 7.5%, about 7.6%, about 7.7%, about 7.8%, about 7.9%, about 8.0% w/w. In some embodiments, the lauroyl polyoxyl-32 glycerides is in an amount of about 7.4% w/w. In some embodiments, the lauroyl polyoxyl-32 glycerides is in an amount of about 7.5% w/w. In some embodiments, the lauroyl polyoxyl-32 glycerides is in an amount of about 10% w/w. In some embodiments, the lauroyl polyoxyl-32 glycerides is in an amount of about 15% w/w. In some embodiments, the lauroyl polyoxyl-32 glycerides is in an amount of about 20% w/w.

In some embodiments, the lauroyl polyoxyl-32 glycerides is in an amount from 5.0% to 20% w/w. In some embodiments, the lauroyl polyoxyl-32 glycerides is in an amount from 5.0% to 15% w/w. In some embodiments, the lauroyl polyoxyl-32 glycerides is in an amount from 5.0% to 10% w/w. In some embodiments, the lauroyl polyoxyl-32 glycerides is in an amount from 5.0% to 9.0% w/w. In some embodiments, the lauroyl polyoxyl-32 glycerides is in an amount from 5.0% to 8.0% w/w. In some embodiments, the lauroyl polyoxyl-32 glycerides is in an amount from 5.0% to 7.5% w/w. In some embodiments, the lauroyl polyoxyl-32 glycerides in an amount from 6.0% to 10% w/w. In some embodiments, the lauroyl polyoxyl-32 glycerides is in an amount from 6.0% to 9.0% w/w. In some embodiments, the lauroyl polyoxyl-32 glycerides is in an amount from 6.0% to 8.0% w/w. In some embodiments, the lauroyl polyoxyl-32 glycerides is in an amount from 6.0% to 7.5% w/w. In some embodiments, the lauroyl polyoxyl-32 glycerides is in an amount from 7.0% to 10.0% w/w. In some embodiments, the lauroyl polyoxyl-32 glycerides is in an amount from 7.0% to 9.0% w/w. In some embodiments, the lauroyl polyoxyl-32 glycerides in an amount from 7.0% to 8.0% w/w. In some embodiments, the lauroyl polyoxyl-32 glycerides is in an amount from 7.0% to 7.5% w/w. In some embodiments, the lauroyl polyoxyl-32 glycerides is in an amount of 7.0%, 7.1%, 7.2%, 7.3%, 7.4%, 7.5%, 7.6%, 7.7%, 7.8%, 7.9%, 8.0% w/w. In some embodiments, the lauroyl polyoxyl-32 glycerides is in an amount of 7.4% w/w. In some embodiments, the lauroyl polyoxyl-32 glycerides is in an amount of 7.5% w/w. In some embodiments, the lauroyl polyoxyl-32 glycerides is in an amount of 10% w/w. In some embodiments, the lauroyl polyoxyl-32 glycerides is in an amount of 15% w/w. In some embodiments, the lauroyl polyoxyl-32 glycerides is in an amount of 20% w/w.

In some embodiments, the pharmaceutical composition further comprises a surfactant (e.g., vitamin E TPGS). In some embodiments, the surfactant is in an amount from about 10% to about 30% w/w. In some embodiments, the surfactant is in an amount from 10% to about 25% w/w. In some embodiments, the surfactant is in an amount from about 10% to about 20% w/w. In some embodiments, the surfactant is in an amount from about 15% to about 30% w/w. In some embodiments, the surfactant is in an amount from about 15% to about 25% w/w. In some embodiments, the surfactant is in an amount from about 15% to about 20% w/w. In some embodiments, the surfactant is in an amount of about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% w/w. In some embodiments, the surfactant is in an amount of about 19.5%, about 19.6%, about 19.7%, about 19.8%, about 19.9%, about 20.1%, about 20.2%, about 20.3%, about 20.4%, about 20.5% w/w. In some embodiments, the surfactant is in an amount of about 19.9% w/w. In some embodiments, the surfactant is in an amount of about 20.0% w/w. In some embodiments, the surfactant is in an amount of about 20.1% w/w.

In some embodiments, the pharmaceutical composition further comprises a surfactant (e.g., vitamin E TPGS). In some embodiments, the surfactant is in an amount from 10% to 30% w/w. In some embodiments, the surfactant is in an amount from 10% to 25% w/w. In some embodiments, the surfactant is in an amount from 10% to 20% w/w. In some embodiments, the surfactant is in an amount from 15% to 30% w/w. In some embodiments, the surfactant is in an amount from 15% to 25% w/w. In some embodiments, the surfactant is in an amount from 15% to 20% w/w. In some embodiments, the surfactant is in an amount of 10%, 11%, 12%, 13%, 14%, 15%, 16%, 17%, 18%, 19%, 20%, 21%, 22%, 23%, 24%, 25%, 26%, 27%, 28%, 29%, or 30% w/w. In some embodiments, the surfactant is in an amount of 19.5%, 19.6%, 19.7%, 19.8%, 19.9%, 20.1%, 20.2%, 20.3%, 20.4%, 20.5% w/w. In some embodiments, the surfactant is in an amount of 19.9% w/w. In some embodiments, the surfactant is in an amount of 20.0% w/w. In some embodiments, the surfactant is in an amount of 20.1% w/w.

In some embodiments, the pharmaceutical composition further comprises vitamin E TPGS. In some embodiments, the vitamin E TPGS is in an amount from about 10% to about 30% w/w. In some embodiments, the vitamin E TPGS is in an amount from 10% to about 25% w/w. In some embodiments, the vitamin E TPGS is in an amount from about 10% to about 20% w/w. In some embodiments, the vitamin E TPGS is in an amount from about 15% to about 30% w/w. In some embodiments, the vitamin E TPGS is in an amount from about 15% to about 25% w/w. In some embodiments, the vitamin E TPGS is in an amount from about 15% to about 20% w/w. In some embodiments, the vitamin E TPGS is in an amount of about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or 30% w/w. In some embodiments, the vitamin E TPGS is in an amount of about 19.5%, about 19.6%, about 19.7%, about 19.8%, about 19.9%, about 20.1%, about 20.2%, about 20.3%, about 20.4%, about 2.5% w/w. In some embodiments, the vitamin E TPGS is in an amount of about 19.9% w/w. In some embodiments, the vitamin E TPGS is in an amount of about 2.0% w/w. In some embodiments, the vitamin E TPGS is in an amount of about 20.1% w/w.

In some embodiments, the vitamin E TPGS is in an amount from 10% to 30% w/w. In some embodiments, the vitamin E TPGS is in an amount from 10% to 25% w/w. In some embodiments, the vitamin E TPGS is in an amount from 10% to 20% w/w. In some embodiments, the vitamin E TPGS is in an amount from 15% to 30% w/w. In some embodiments, the vitamin E TPGS is in an amount from 15% to 25% w/w. In some embodiments, the vitamin E TPGS is in an amount from 15% to 20% w/w. In some embodiments, the vitamin E TPGS is in an amount of 10%, 11%, 12%, 13%, 14%, 15%, 16%, 17%, 18%, 19%, 20%, 21%, 22%, 23%, 24%, 25%, 26%, 27%, 28%, 29%, or 30% w/w. In some embodiments, the vitamin E TPGS is in an amount of 19.5%, 19.6%, 19.7%, 19.8%, 19.9%, 2.1%, 2.2%, 2.3%, 2.4%, 2.5% w/w. In some embodiments, the vitamin E TPGS is in an amount of 19.9% w/w. In some embodiments, the vitamin E TPGS is in an amount of 20.0% w/w. In some embodiments, the vitamin E TPGS is in an amount of 20.1% w/w.

In some embodiments, the pharmaceutical composition further comprises an antioxidant (e.g., butylated hydroxyanisole (BHA), butylated hydroxytoluene (BHT), tocopherol, tocopherol acetate, etc.).

In some embodiments, the antioxidant is an amount from about 0.005% to about 0.015% w/w. In some embodiments, the antioxidant is an amount from about 0.005% to about 0.010% w/w. In some embodiments, the antioxidant is an amount from about 0.010% to about 0.015% w/w. In some embodiments, the antioxidant is an amount from about 0.008% to about 0.012% w/w. In some embodiments, the antioxidant is an amount of about 0.005%, about 0.006%, about 0.007%, about 0.008%, about 0.009%, about 0.010%, about 0.011%, about 0.012%, about 0.013%, about 0.014%, about 0.015% w/w. In some embodiments, the antioxidant is an amount of about 0.008% w/w. In some embodiments, the antioxidant is an amount of about 0.009% w/w. In some embodiments, the antioxidant is an amount of about 0.010% w/w. In some embodiments, the antioxidant is an amount of about 0.011% w/w. In some embodiments, the antioxidant is an amount of about 0.012% to about 0.015% w/w.

In some embodiments the pharmaceutical composition further comprises an antioxidant such as butylated hydroxyanisole (BHA). In some embodiments, the antioxidant is an amount from 0.005% to 0.015% w/w. In some embodiments, the antioxidant is an amount from 0.005% to 0.010% w/w. In some embodiments, the antioxidant is an amount from 0.010% to 0.015% w/w. In some embodiments, the antioxidant is an amount from 0.008% to 0.012% w/w. In some embodiments, the antioxidant is an amount of 0.005%, 0.006%, 0.007%, 0.008%, 0.009%, 0.010%, 0.011%, 0.012%, 0.013%, 0.014%, 0.015% w/w. In some embodiments, the antioxidant is an amount of 0.008% w/w. In some embodiments, the antioxidant is an amount of 0.009% w/w. In some embodiments, the antioxidant is an amount of 0.010% w/w. In some embodiments, the antioxidant is an amount of 0.011% w/w. In some embodiments, the antioxidant is an amount of 0.012% to 0.015% w/w.

In some embodiments, the butylated hydroxyanisole (BHA) is an amount from about 0.005% to about 0.015% w/w. In some embodiments, butylated hydroxyanisole (BHA) is an amount from about 0.005% to about 0.010% w/w. In some embodiments, butylated hydroxyanisole (BHA) is an amount from about 0.010% to about 0.015% w/w. In some embodiments, butylated hydroxyanisole (BHA) is an amount from about 0.008% to about 0.012% w/w. In some embodiments, butylated hydroxyanisole (BHA) is an amount of about 0.005%, about 0.006%, about 0.007%, about 0.008%, about 0.009%, about 0.010%, about 0.011%, about 0.012%, about 0.013%, about 0.014%, about 0.015% w/w. In some embodiments, butylated hydroxyanisole (BHA) is an amount of about 0.008% w/w. In some embodiments, butylated hydroxyanisole (BHA) is an amount of about 0.009% w/w. In some embodiments, butylated hydroxyanisole (BHA) is an amount of about 0.010% w/w. In some embodiments, butylated hydroxyanisole (BHA) is an amount of about 0.011% w/w. In some embodiments, butylated hydroxyanisole (BHA) is an amount of about 0.012% to about 0.015% w/w.

In some embodiments the pharmaceutical composition further comprises an antioxidant such as butylated hydroxyanisole (BHA). In some embodiments, the butylated hydroxyanisole (BHA) is an amount from 0.005% to 0.015% w/w. In some embodiments, butylated hydroxyanisole (BHA) is an amount from 0.005% to 0.010% w/w. In some embodiments, butylated hydroxyanisole (BHA) is an amount from 0.010% to 0.015% w/w. In some embodiments, butylated hydroxyanisole (BHA) is an amount from 0.008% to 0.012% w/w. In some embodiments, butylated hydroxyanisole (BHA) is an amount of 0.005%, 0.006%, 0.007%, 0.008%, 0.009%, 0.010%, 0.011%, 0.012%, 0.013%, 0.014%, 0.015% w/w. In some embodiments, butylated hydroxyanisole (BHA) is an amount of 0.008% w/w. In some embodiments, butylated hydroxyanisole (BHA) is an amount of 0.009% w/w. In some embodiments, butylated hydroxyanisole (BHA) is an amount of 0.010% w/w. In some embodiments, butylated hydroxyanisole (BHA) is an amount of 0.011% w/w. In some embodiments, butylated hydroxyanisole (BHA) is an amount of 0.012% to 0.015% w/w.

In some embodiments, the pharmaceutical composition comprises about 0.3% w/w Compound I, about 60% w/w glyceryl monocaprylate, about 20% w/w glyceryl monolinoleate, about 20% w/w vitamin E TPGS, and about 0.01% w/w butylated hydroxyanisole. In some embodiments, the pharmaceutical composition comprises 0.3% w/w Compound I, 60% w/w glyceryl monocaprylate, 20% w/w glyceryl monolinoleate, 20% w/w vitamin E TPGS, and 0.01% w/w butylated hydroxyanisole. In some embodiments, the pharmaceutical composition comprises 0.3% Compound I, 59.8% glyceryl monocaprylate, 19.9% glyceryl monolinoleate, 19.9% vitamin E TPGS, 0.01% butylated hydroxyanisole.

In some embodiments, the pharmaceutical composition comprises about 0.3% w/w Compound I, about 50% w/w glyceryl monocaprylate, about 22.5% w/w glyceryl monolinoleate, about 20% w/w vitamin E TPGS, about 7.5% w/w stearoyl polyoxyl-32 glyceride, and about 0.01% w/w butylated hydroxyanisole. In some embodiments, the pharmaceutical composition comprises 0.3% w/w Compound I, 50% w/w glyceryl monocaprylate, 22.5% w/w glyceryl monolinoleate, 20% w/w vitamin E TPGS, 7.5% w/w stearoyl polyoxyl-32 glyceride, and 0.01% w/w butylated hydroxyanisole. In some embodiments, the pharmaceutical composition comprises about 0.3% w/w Compound I, 49.8% w/w glyceryl monocaprylate, 22.4% w/w glyceryl monolinoleate, 19.9% w/w vitamin E TPGS, 7.5% w/w stearoyl polyoxyl-32 glyceride, and 0.01% w/w butylated hydroxyanisole.

In some embodiments, the pharmaceutical composition is an oral dosage form. In some embodiments, the pharmaceutical composition is a lipid formulation. In some embodiments, the oral dosage form is a soft gelatin capsule, a hardshell capsule or an oral liquid. In some embodiments, the oral dosage form is a soft gelatin capsule. In some embodiments, the oral dosage form is a hardshell capsule. In some embodiments, the oral dosage form is an oral liquid.

In one aspect, the disclosure provides a pharmaceutical composition comprising a therapeutically effective amount of Compound I

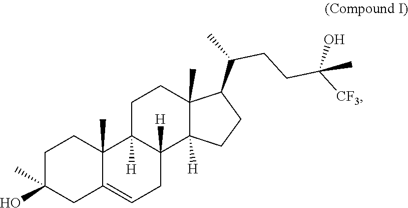

or a pharmaceutically acceptable salt thereof, and a pharmaceutically acceptable carrier; wherein following administration of the pharmaceutical composition to a subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.

In some embodiments, the pharmaceutical composition provides a Cₘₐₓ of between about 8 and about 25 ng/mL. In some embodiments, the pharmaceutical composition provides a Cₘₐₓ of between about 9 and about 25 ng/mL. In some embodiments, the pharmaceutical composition provides a Cₘₐₓ of between about 10 and about 25 ng/mL. In some embodiments, the pharmaceutical composition provides a Cₘₐₓ of between about 11 and about 25 ng/mL. In some embodiments, the pharmaceutical composition provides a Cₘₐₓ of between about 12 and about 25 ng/mL. In some embodiments, the pharmaceutical composition provides a Cₘₐₓ of between about 13 and about 25 ng/mL. In some embodiments, the pharmaceutical composition provides a Cₘₐₓ of between about 14 and about 25 ng/mL. In some embodiments, the pharmaceutical composition provides a Cₘₐₓ of between about 15 and about 25 ng/mL.

In some embodiments, the pharmaceutical composition provides a Cₘₐₓ of about 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23.0, 23.5, 24, 24.5 or 25 ng/mL. In some embodiments, the pharmaceutical composition provides a Cₘₐₓ of about 12, 12.1, 12.2, 12.3, 12.4, 12.6, 12.7, 12.8, 12.9, 13.0 ng/mL. 15.1, 15.2, 15.3, 15.4, 15.6, 15.7, 15.8, 15.9, or 16 ng/mL. In some embodiments, the pharmaceutical composition provides a Cₘₐₓ of about 12 ng/mL. In some embodiments, the pharmaceutical composition provides a Cₘₐₓ of about 12.1 ng/mL. In some embodiments, the pharmaceutical composition provides a Cₘₐₓ of about 12.2 ng/mL. In some embodiments, the pharmaceutical composition provides a Cₘₐₓ of about 12.3 ng/mL. In some embodiments, the pharmaceutical composition provides a Cₘₐₓ of about 12.4 ng/mL. In some embodiments, the pharmaceutical composition provides a Cₘₐₓ of about 12.5 ng/mL. In some embodiments, the pharmaceutical composition provides a Cₘₐₓ of about 12.6 ng/mL. In some embodiments, the pharmaceutical composition provides a Cₘₐₓ of about 12.7 ng/mL. In some embodiments, the pharmaceutical composition provides a Cₘₐₓ of about 12.8 ng/mL. In some embodiments, the pharmaceutical composition provides a Cₘₐₓ of about 12.9 ng/mL. In some embodiments, the pharmaceutical composition provides a Cₘₐₓ of about 13 ng/mL. In some embodiments, the pharmaceutical composition provides a Cₘₐₓ of about 15 ng/mL. In some embodiments, the pharmaceutical composition provides a Cₘₐₓ of about 15.1 ng/mL. In some embodiments, the pharmaceutical composition provides a Cₘₐₓ of about 15.2 ng/mL. In some embodiments, the pharmaceutical composition provides a Cₘₐₓ of about 15.3 ng/mL. In some embodiments, the pharmaceutical composition provides a Cₘₐₓ of about 15.4 ng/mL. In some embodiments, the pharmaceutical composition provides a Cₘₐₓ of about 15.5 ng/mL. In some embodiments, the pharmaceutical composition provides a Cₘₐₓ of about 15.6 ng/mL. In some embodiments, the pharmaceutical composition provides a Cₘₐₓ of about 15.7 ng/mL. In some embodiments, the pharmaceutical composition provides a Cₘₐₓ of about 15.8 ng/mL. In some embodiments, the pharmaceutical composition provides a Cₘₐₓ of about 15.9 ng/mL. In some embodiments, the pharmaceutical composition provides a Cₘₐₓ of about 16 ng/mL.

In some embodiments, the pharmaceutical composition provides a Cₘₐₓ of between 8 and 25 ng/mL. In some embodiments, the pharmaceutical composition provides a Cₘₐₓ of between 9 and 25 ng/mL. In some embodiments, the pharmaceutical composition provides a Cₘₐₓ of between 10 and 25 ng/mL. In some embodiments, the pharmaceutical composition provides a Cₘₐₓ of between 11 and 25 ng/mL. In some embodiments, the pharmaceutical composition provides a Cₘₐₓ of between 12 and 25 ng/mL. In some embodiments, the pharmaceutical composition provides a Cₘₐₓ of between 13 and 25 ng/mL. In some embodiments, the pharmaceutical composition provides a Cₘₐₓ of between 14 and 25 ng/mL. In some embodiments, the pharmaceutical composition provides a Cₘₐₓ of between 15 and 25 ng/mL.

In some embodiments, the pharmaceutical composition provides a Cₘₐₓ of 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23.0, 23.5, 24, 24.5 or 25 ng/mL. In some embodiments, the pharmaceutical composition provides a Cₘₐₓ of 12, 12.1, 12.2, 12.3, 12.4, 12.6, 12.7, 12.8, 12.9, 13.0 ng/mL. In some embodiments, the pharmaceutical composition provides a Cₘₐₓ of 15.1, 15.2, 15.3, 15.4, 15.6, 15.7, 15.8, 15.9, or 16 ng/mL. In some embodiments, the pharmaceutical composition provides a Cₘₐₓ of 12 ng/mL. In some embodiments, the pharmaceutical composition provides a Cₘₐₓ of 12.1 ng/mL. In some embodiments, the pharmaceutical composition provides a Cₘₐₓ of 12.2 ng/mL. In some embodiments, the pharmaceutical composition provides a Cₘₐₓ of 12.3 ng/mL. In some embodiments, the pharmaceutical composition provides a Cₘₐₓ of 12.4 ng/mL. In some embodiments, the pharmaceutical composition provides a Cₘₐₓ of 12.5 ng/mL. In some embodiments, the pharmaceutical composition provides a Cₘₐₓ of 12.6 ng/mL. In some embodiments, the pharmaceutical composition provides a Cₘₐₓ of 12.7 ng/mL. In some embodiments, the pharmaceutical composition provides a Cₘₐₓ of 12.8 ng/mL. In some embodiments, the pharmaceutical composition provides a Cₘₐₓ of 12.9 ng/mL. In some embodiments, the pharmaceutical composition provides a Cₘₐₓ of 13 ng/mL. In some embodiments, the pharmaceutical composition provides a Cₘₐₓ of 15 ng/mL. In some embodiments, the pharmaceutical composition provides a Cₘₐₓ of 15.1 ng/mL. In some embodiments, the pharmaceutical composition provides a Cₘₐₓ of 15.2 ng/mL. In some embodiments, the pharmaceutical composition provides a Cₘₐₓ of 15.3 ng/mL. In some embodiments, the pharmaceutical composition provides a Cₘₐₓ of 15.4 ng/mL. In some embodiments, the pharmaceutical composition provides a Cₘₐₓ of 15.5 ng/mL. In some embodiments, the pharmaceutical composition provides a Cₘₐₓ of 15.6 ng/mL. In some embodiments, the pharmaceutical composition provides a Cₘₐₓ of 15.7 ng/mL. In some embodiments, the pharmaceutical composition provides a Cₘₐₓ of 15.8 ng/mL. In some embodiments, the pharmaceutical composition provides a Cₘₐₓ of 15.9 ng/mL. In some embodiments, the pharmaceutical composition provides a Cₘₐₓ of 16 ng/mL.

In some embodiments, the Cₘₐₓ is a geometric mean Cₘₐₓ.

In some embodiments, the range of Cₘₐₓ is a range of geometric mean Cₘₐₓ.

In some embodiments, the pharmaceutical composition provides a tₘₐₓ of between about 2 and about 8 hours. In some embodiments, the pharmaceutical composition provides a tₘₐₓ of between about 3 and about 8 hours. In some embodiments, the pharmaceutical composition provides a tₘₐₓ of between about 4 and about 8 hours. In some embodiments, the pharmaceutical composition provides a tₘₐₓ of between about 5 and about 8 hours. In some embodiments, the pharmaceutical composition provides a tₘₐₓ of between about 6 and about 8 hours. In some embodiments, the pharmaceutical composition provides a tₘₐₓ of between about 7 and about 8 hours. In some embodiments, the pharmaceutical composition provides a tₘₐₓ of between about 3 and about 6 hours. In some embodiments, the pharmaceutical composition provides a tₘₐₓ of about 2 hours. In some embodiments, the pharmaceutical composition provides a tₘₐₓ of about 2.5 hours. In some embodiments, the pharmaceutical composition provides a tₘₐₓ of about 3 hours. In some embodiments, the pharmaceutical composition provides a tₘₐₓ of about 3.5 hours. In some embodiments, the pharmaceutical composition provides a tₘₐₓ of about 4 hours. In some embodiments, the pharmaceutical composition provides a tₘₐₓ of about 4.5 hours. In some embodiments, the pharmaceutical composition provides a tₘₐₓ of about 5 hours. In some embodiments, the pharmaceutical composition provides a tₘₐₓ of about 5.5 hours. In some embodiments, the pharmaceutical composition provides a tₘₐₓ of about 6 hours. In some embodiments, the pharmaceutical composition provides a tₘₐₓ of about 6.5 hours. In some embodiments, the pharmaceutical composition provides a tₘₐₓ of about 7 hours. In some embodiments, the pharmaceutical composition provides a tₘₐₓ of about 7.5 hours. In some embodiments, the pharmaceutical composition provides a tₘₐₓ of about 8 hours.

In some embodiments, the pharmaceutical composition provides a tₘₐₓ of between 2 and 8 hours. In some embodiments, the pharmaceutical composition provides a tₘₐₓ of between 3 and 8 hours. In some embodiments, the pharmaceutical composition provides a tₘₐₓ of between 4 and 8 hours. In some embodiments, the pharmaceutical composition provides a tₘₐₓ of between 5 and 8 hours. In some embodiments, the pharmaceutical composition provides a tₘₐₓ of between 6 and 8 hours. In some embodiments, the pharmaceutical composition provides a tₘₐₓ of between 7 and 8 hours. In some embodiments, the pharmaceutical composition provides a tₘₐₓ of between 3 and 6 hours. In some embodiments, the pharmaceutical composition provides a tₘₐₓ of 2 hours. In some embodiments, the pharmaceutical composition provides a tₘₐₓ of 2.5 hours. In some embodiments, the pharmaceutical composition provides a tₘₐₓ of 3 hours. In some embodiments, the pharmaceutical composition provides a tₘₐₓ of 3.5 hours. In some embodiments, the pharmaceutical composition provides a tₘₐₓ of 4 hours. In some embodiments, the pharmaceutical composition provides a tₘₐₓ of 4.5 hours. In some embodiments, the pharmaceutical composition provides a tₘₐₓ of 5 hours. In some embodiments, the pharmaceutical composition provides a tₘₐₓ of 5.5 hours. In some embodiments, the pharmaceutical composition provides a tₘₐₓ of 6 hours. In some embodiments, the pharmaceutical composition provides a tₘₐₓ of 6.5 hours. In some embodiments, the pharmaceutical composition provides a tₘₐₓ of 7 hours. In some embodiments, the pharmaceutical composition provides a tₘₐₓ of 7.5 hours. In some embodiments, the pharmaceutical composition provides a tₘₐₓ of 8 hours.

In some embodiments, the tₘₐₓ is a geometric mean tₘₐₓ.

In some embodiments, the range of tₘₐₓ is a range of geometric mean tₘₐₓ.

In some embodiments, the pharmaceutical composition provides a tₗₐₛₜ of between about 20 and about 200 hours. In some embodiments, the pharmaceutical composition provides a tₗₐₛₜ of between about 20, about 24, about 28, about 32, about 36, about 40, about 44, about 48, about 52, about 56, about 60, about 64, about 68, about 72, about 76, about 80, about 84, about 88, about 92, about 96, about 100, about 104, about 108, about 112, about 116, about 120, about 124, about 128, about 132, about 136, about 140, about 144, about 148, about 152, about 156, about 160, about 164, about 168, about 172, about 176, about 180, about 184, about 188, about 192, about 196, or about 200 hours.

In some embodiments, the pharmaceutical composition provides a tₗₐₛₜ of between 20 and 200 hours. In some embodiments, the pharmaceutical composition provides a tₗₐₛₜ of between 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, or 200 hours.

In some embodiments, the tₗₐₛₜ is a geometric mean tₗₐₛₜ.

In some embodiments, the range of tₗₐₛₜ is a range of geometric mean tₗₐₛₜ.

In some embodiments, the pharmaceutical composition provides an AUCₗₐₛₜ of between about 100 and about 450 ng*h/mL. In some embodiments, the pharmaceutical composition provides an AUCₗₐₛₜ of between about 125 and about 400 ng*h/mL. In some embodiments, the pharmaceutical composition provides an AUCₗₐₛₜ of between about 150 and about 350 ng*h/mL. In some embodiments, the pharmaceutical composition provides an AUCₗₐₛₜ of between about 175 and about 300 ng*h/mL. In some embodiments, the pharmaceutical composition provides an AUCₗₐₛₜ of between about 200 and about 275 ng*h/mL. In some embodiments, the pharmaceutical composition provides an AUCₗₐₛₜ of between about 225 and about 250 ng*h/mL.

In some embodiments, the pharmaceutical composition provides an AUCₗₐₛₜ of about 225, about 226, about 227, about 228, about 229, about 230, about 231, about 232, about 233, about 234, about 235, about 236, about 237, about 238, about 239, about 240, about 241, about 242, about 243, about 245, about 246, about 247, about 248, about 249, or about 250 ng*h/mL. In some embodiments, the pharmaceutical composition provides an AUCₗₐₛₜ of about 225 ng*h/mL. In some embodiments, the pharmaceutical composition provides an AUCₗₐₛₜ of about 226 ng*h/mL. In some embodiments, the pharmaceutical composition provides an AUCₗₐₛₜ of about 227 ng*h/mL. In some embodiments, the pharmaceutical composition provides an AUCₗₐₛₜ of about 228 ng*h/mL. In some embodiments, the pharmaceutical composition provides an AUCₗₐₛₜ of about 229 ng*h/mL. In some embodiments, the pharmaceutical composition provides an AUCₗₐₛₜ of about 230 ng*h/mL. In some embodiments, the pharmaceutical composition provides an AUCₗₐₛₜ of about 231 ng*h/mL. In some embodiments, the pharmaceutical composition provides an AUCₗₐₛₜ of about 232 ng*h/mL. In some embodiments, the pharmaceutical composition provides an AUCₗₐₛₜ of about 233 ng*h/mL. In some embodiments, the pharmaceutical composition provides an AUCₗₐₛₜ of about 234 ng*h/mL. In some embodiments, the pharmaceutical composition provides an AUCₗₐₛₜ of about 235 ng*h/mL. In some embodiments, the pharmaceutical composition provides an AUCₗₐₛₜ of about 236 ng*h/mL. In some embodiments, the pharmaceutical composition provides an AUCₗₐₛₜ of about 237 ng*h/mL. In some embodiments, the pharmaceutical composition provides an AUCₗₐₛₜ of about 238 ng*h/mL. In some embodiments, the pharmaceutical composition provides an AUCₗₐₛₜ of about 239 ng*h/mL. In some embodiments, the pharmaceutical composition provides an AUCₗₐₛₜ of about ng*h/mL. In some embodiments, the pharmaceutical composition provides an AUCₗₐₛₜ of about 241 ng*h/mL. In some embodiments, the pharmaceutical composition provides an AUCₗₐₛₜ of about 242 ng*h/mL. In some embodiments, the pharmaceutical composition provides an AUCₗₐₛₜ of about 243 ng*h/mL. In some embodiments, the pharmaceutical composition provides an AUCₗₐₛₜ of about 244 ng*h/mL. In some embodiments, the pharmaceutical composition provides an AUCₗₐₛₜ of about 245 ng*h/mL. In some embodiments, the pharmaceutical composition provides an AUCₗₐₛₜ of about 246 ng*h/mL. In some embodiments, the pharmaceutical composition provides an AUCₗₐₛₜ of about 247 ng*h/mL. In some embodiments, the pharmaceutical composition provides an AUCₗₐₛₜ of about 248 ng*h/mL. In some embodiments, the pharmaceutical composition provides an AUCₗₐₛₜ of about 249 ng*h/mL. In some embodiments, the pharmaceutical composition provides an AUCₗₐₛₜ of about 250 ng*h/mL.

In some embodiments, the pharmaceutical composition provides an AUCₗₐₛₜ of between 100 and 450 ng*h/mL. In some embodiments, the pharmaceutical composition provides an AUCₗₐₛₜ of between 125 and 400 ng*h/mL. In some embodiments, the pharmaceutical composition provides an AUCₗₐₛₜ of between 150 and 350 ng*h/mL. In some embodiments, the pharmaceutical composition provides an AUCₗₐₛₜ of between 175 and 300 ng*h/mL. In some embodiments, the pharmaceutical composition provides an AUCₗₐₛₜ of between 200 and 275 ng*h/mL. In some embodiments, the pharmaceutical composition provides an AUCₗₐₛₜ of between 225 and 250 ng*h/mL.

In some embodiments, the pharmaceutical composition provides an AUCₗₐₛₜ of 225, 226, 227, 228, 229, 230, 231, 232, 233, 234, 235, 236, 237, 238, 239, 240, 241, 242, 243, 245, 246, 247, 248, 249, 250 ng*h/mL. In some embodiments, the pharmaceutical composition provides an AUCₗₐₛₜ of 225 ng*h/mL. In some embodiments, the pharmaceutical composition provides an AUCₗₐₛₜ of 226 ng*h/mL. In some embodiments, the pharmaceutical composition provides an AUCₗₐₛₜ of 227 ng*h/mL. In some embodiments, the pharmaceutical composition provides an AUCₗₐₛₜ of 228 ng*h/mL. In some embodiments, the pharmaceutical composition provides an AUCₗₐₛₜ of 229 ng*h/mL. In some embodiments, the pharmaceutical composition provides an AUCₗₐₛₜ of 230 ng*h/mL. In some embodiments, the pharmaceutical composition provides an AUCₗₐₛₜ of 231 ng*h/mL. In some embodiments, the pharmaceutical composition provides an AUCₗₐₛₜ of 232 ng*h/mL. In some embodiments, the pharmaceutical composition provides an AUCₗₐₛₜ of 233 ng*h/mL. In some embodiments, the pharmaceutical composition provides an AUCₗₐₛₜ of 234 ng*h/mL. In some embodiments, the pharmaceutical composition provides an AUCₗₐₛₜ of 235 ng*h/mL. In some embodiments, the pharmaceutical composition provides an AUCₗₐₛₜ of 236 ng*h/mL. In some embodiments, the pharmaceutical composition provides an AUCₗₐₛₜ of 237 ng*h/mL. In some embodiments, the pharmaceutical composition provides an AUCₗₐₛₜ of 238 ng*h/mL. In some embodiments, the pharmaceutical composition provides an AUCₗₐₛₜ of 239 ng*h/mL. In some embodiments, the pharmaceutical composition provides an AUCₗₐₛₜ of 240 ng*h/mL. In some embodiments, the pharmaceutical composition provides an AUCₗₐₛₜ of 241 ng*h/mL. In some embodiments, the pharmaceutical composition provides an AUCₗₐₛₜ of 242 ng*h/mL. In some embodiments, the pharmaceutical composition provides an AUCₗₐₛₜ of 243 ng*h/mL. In some embodiments, the pharmaceutical composition provides an AUCₗₐₛₜ of 244 ng*h/mL. In some embodiments, the pharmaceutical composition provides an AUCₗₐₛₜ of 245 ng*h/mL. In some embodiments, the pharmaceutical composition provides an AUCₗₐₛₜ of 246 ng*h/mL. In some embodiments, the pharmaceutical composition provides an AUCₗₐₛₜ of 247 ng*h/mL. In some embodiments, the pharmaceutical composition provides an AUCₗₐₛₜ of 248 ng*h/mL. In some embodiments, the pharmaceutical composition provides an AUCₗₐₛₜ of 249 ng*h/mL. In some embodiments, the pharmaceutical composition provides an AUCₗₐₛₜ of 250 ng*h/mL.

In some embodiments, the AUCₗₐₛₜ is a geometric mean AUCₗₐₛₜ.

In some embodiments, the range of AUCₗₐₛₜ is a range of geometric mean AUCₗₐₛₜ.

In some embodiments, the pharmaceutical composition provides an AUCinf of between about 100 and about 500 ng*h/mL. In some embodiments, the pharmaceutical composition provides an AUC_{Inf} of between about 125 and about 400 ng*h/mL. In some embodiments, the pharmaceutical composition provides an AUCₕ f of between about 150 and about 350 ng*h/mL. In some embodiments, the pharmaceutical composition provides an AUC_{Inf} of between about 175 and about 300 ng*h/mL. In some embodiments, the pharmaceutical composition provides an AUC_{Inf} of between about 200 and about 275 ng*h/mL. In some embodiments, the pharmaceutical composition provides an AUC_{Inf} of between about 225 and about 275 ng*h/mL. In some embodiments, the pharmaceutical composition provides an AUC_{Inf} of between about 250 and about 275 ng*h/mL.

In some embodiments, the pharmaceutical composition provides an AUC_{Inf} of about 225, about 226, about 227, about 228, about 229, about 230, about 231, about 232, about 233, about 234, about 235, about 236, about 237, about 238, about 239, about 240, about 241, about 242, about 243, about 245, about 246, about 247, about 248, about 249, about 250, about 251, about 252, about 253, about 254, about 255, about 256, about 257, about 258, about 259, about 260, about 261, about 262, about 263, about 265, about 266, about 267, about 268, about 269, about 270, about 271, about 272, about 273, or about 265 ng*h/mL. In some embodiments, the pharmaceutical composition provides an AUC_{Inf} of about 225 ng*h/mL. In some embodiments, the pharmaceutical composition provides an AUC_{Inf} of about 226 ng*h/mL. In some embodiments, the pharmaceutical composition provides an AUC_{Inf} of about 227 ng*h/mL. In some embodiments, the pharmaceutical composition provides an AUC_{Inf} of about 228 ng*h/mL. In some embodiments, the pharmaceutical composition provides an AUC_{Inf} of about 229 ng*h/mL. In some embodiments, the pharmaceutical composition provides an AUC_{Inf} of about 230 ng*h/mL. In some embodiments, the pharmaceutical composition provides an AUC_{Inf} of about 231 ng*h/mL. In some embodiments, the pharmaceutical composition provides an AUC_{Inf} of about 232 ng*h/mL. In some embodiments, the pharmaceutical composition provides an AUC_{Inf} of about 233 ng*h/mL. In some embodiments, the pharmaceutical composition provides an AUC_{Inf} of about 234 ng*h/mL. In some embodiments, the pharmaceutical composition provides an AUC_{Inf} of about 235 ng*h/mL. In some embodiments, the pharmaceutical composition provides an AUC_{Inf} of about 236 ng*h/mL. In some embodiments, the pharmaceutical composition provides an AUC_{Inf} of about 237 ng*h/mL. In some embodiments, the pharmaceutical composition provides an AUC_{Inf} of about 238 ng*h/mL. In some embodiments, the pharmaceutical composition provides an AUC_{Inf} of about 239 ng*h/mL. In some embodiments, the pharmaceutical composition provides an AUC_{Inf} of about ng*h/mL. In some embodiments, the pharmaceutical composition provides an AUC_{Inf} of about 241 ng*h/mL. In some embodiments, the pharmaceutical composition provides an AUC_{Inf} of about 242 ng*h/mL. In some embodiments, the pharmaceutical composition provides an AUC_{Inf} of about 243 ng*h/mL. In some embodiments, the pharmaceutical composition provides an AUC_{Inf} of about 244 ng*h/mL. In some embodiments, the pharmaceutical composition provides an AUC_{Inf} of about 245 ng*h/mL. In some embodiments, the pharmaceutical composition provides an AUC_{Inf} of about 246 ng*h/mL. In some embodiments, the pharmaceutical composition provides an AUC_{Inf} of about 247 ng*h/mL. In some embodiments, the pharmaceutical composition provides an AUC_{Inf} of about 248 ng*h/mL. In some embodiments, the pharmaceutical composition provides an AUC_{Inf} of about 249 ng*h/mL. In some embodiments, the pharmaceutical composition provides an AUC_{Inf} of about 250 ng*h/mL. In some embodiments, the pharmaceutical composition provides an AUC_{Inf} of about 251 ng*h/mL. In some embodiments, the pharmaceutical composition provides an AUC_{Inf} of about 252 ng*h/mL. In some embodiments, the pharmaceutical composition provides an AUC_{Inf} of about 253 ng*h/mL. In some embodiments, the pharmaceutical composition provides an AUC_{Inf} of about 254 ng*h/mL. In some embodiments, the pharmaceutical composition provides an AUC_{Inf} of about 255 ng*h/mL. In some embodiments, the pharmaceutical composition provides an AUC_{Inf} of about 256 ng*h/mL. In some embodiments, the pharmaceutical composition provides an AUC_{Inf} of about 257 ng*h/mL. In some embodiments, the pharmaceutical composition provides an AUC_{Inf} of about 258 ng*h/mL. In some embodiments, the pharmaceutical composition provides an AUC_{Inf} of about 259 ng*h/mL. In some embodiments, the pharmaceutical composition provides an AUC_{Inf} of about 260 ng*h/mL. In some embodiments, the pharmaceutical composition provides an AUC_{Inf} of about 261 ng*h/mL. In some embodiments, the pharmaceutical composition provides an AUC_{Inf} of about 262 ng*h/mL. In some embodiments, the pharmaceutical composition provides an AUC_{Inf} of about 263 ng*h/mL. In some embodiments, the pharmaceutical composition provides an AUC_{Inf} of about 264 ng*h/mL. In some embodiments, the pharmaceutical composition provides an AUC_{Inf} of about 265 ng*h/mL. In some embodiments, the pharmaceutical composition provides an AUC_{Inf} of about 266 ng*h/mL. In some embodiments, the pharmaceutical composition provides an AUC_{Inf} of about 267 ng*h/mL. In some embodiments, the pharmaceutical composition provides an AUC_{Inf} of about 268 ng*h/mL. In some embodiments, the pharmaceutical composition provides an AUC_{Inf} of about 269 ng*h/mL. In some embodiments, the pharmaceutical composition provides an AUC_{Inf} of about 270 ng*h/mL.

In some embodiments, the pharmaceutical composition provides an AUC_{Inf} of between 100 and 500 ng*h/mL. In some embodiments, the pharmaceutical composition provides an AUC_{Inf} of between 125 and 400 ng*h/mL. In some embodiments, the pharmaceutical composition provides an AUC_{Inf} of between 150 and 350 ng*h/mL. In some embodiments, the pharmaceutical composition provides an AUC_{Inf} of between 175 and 300 ng*h/mL. In some embodiments, the pharmaceutical composition provides an AUC_{Inf} of between 200 and 275 ng*h/mL. In some embodiments, the pharmaceutical composition provides an AUC_{Inf} of between 225 and 275 ng*h/mL. In some embodiments, the pharmaceutical composition provides an AUC_{Inf} of between 250 and 275 ng*h/mL.

In some embodiments, the pharmaceutical composition provides an AUC_{Inf} of 225, 226, 227, 228, 229, 230, 231, 232, 233, 234, 235, 236, 237, 238, 239, 240, 241, 242, 243, 245, 246, 247, 248, 249, 250, 251, 252, 253, 254, 255, 256, 257, 258, 259, 260, 261, 262, 263, 265, 266, 267, 268, 269, 270, 271, 272, 273, or 265 ng*h/mL. In some embodiments, the pharmaceutical composition provides an AUC_{Inf} of 225 ng*h/mL. In some embodiments, the pharmaceutical composition provides an AUC_{Inf} of 226 ng*h/mL. In some embodiments, the pharmaceutical composition provides an AUC_{Inf} of 227 ng*h/mL. In some embodiments, the pharmaceutical composition provides an AUC_{Inf} of 228 ng*h/mL. In some embodiments, the pharmaceutical composition provides an AUC_{Inf} of 229 ng*h/mL. In some embodiments, the pharmaceutical composition provides an AUC_{Inf} of 230 ng*h/mL. In some embodiments, the pharmaceutical composition provides an AUC_{Inf} of 231 ng*h/mL. In some embodiments, the pharmaceutical composition provides an AUC_{Inf} of 232 ng*h/mL. In some embodiments, the pharmaceutical composition provides an AUC_{Inf} of 233 ng*h/mL. In some embodiments, the pharmaceutical composition provides an AUC_{Inf} of 234 ng*h/mL. In some embodiments, the pharmaceutical composition provides an AUC_{Inf} of 235 ng*h/mL. In some embodiments, the pharmaceutical composition provides an AUC_{Inf} of 236 ng*h/mL. In some embodiments, the pharmaceutical composition provides an AUC_{Inf} of 237 ng*h/mL. In some embodiments, the pharmaceutical composition provides an AUC_{Inf} of 238 ng*h/mL. In some embodiments, the pharmaceutical composition provides an AUC_{Inf} of 239 ng*h/mL. In some embodiments, the pharmaceutical composition provides an AUC_{Inf} of 240 ng*h/mL. In some embodiments, the pharmaceutical composition provides an AUC_{Inf} of 241 ng*h/mL. In some embodiments, the pharmaceutical composition provides an AUC_{Inf} of 242 ng*h/mL. In some embodiments, the pharmaceutical composition provides an AUC_{Inf} of 243 ng*h/mL. In some embodiments, the pharmaceutical composition provides an AUC_{Inf} of 244 ng*h/mL. In some embodiments, the pharmaceutical composition provides an AUC_{Inf} of 245 ng*h/mL. In some embodiments, the pharmaceutical composition provides an AUC_{Inf} of 246 ng*h/mL. In some embodiments, the pharmaceutical composition provides an AUC_{Inf} of 247 ng*h/mL. In some embodiments, the pharmaceutical composition provides an AUC_{Inf} of 248 ng*h/mL. In some embodiments, the pharmaceutical composition provides an AUC_{Inf} of 249 ng*h/mL. In some embodiments, the pharmaceutical composition provides an AUC_{Inf} of 250 ng*h/mL. In some embodiments, the pharmaceutical composition provides an AUC_{Inf} of 251 ng*h/mL. In some embodiments, the pharmaceutical composition provides an AUC_{Inf} of 252 ng*h/mL. In some embodiments, the pharmaceutical composition provides an AUC_{Inf} of 253 ng*h/mL. In some embodiments, the pharmaceutical composition provides an AUC_{Inf} of 254 ng*h/mL. In some embodiments, the pharmaceutical composition provides an AUC_{Inf} of 255 ng*h/mL. In some embodiments, the pharmaceutical composition provides an AUC_{Inf} of 256 ng*h/mL. In some embodiments, the pharmaceutical composition provides an AUC_{Inf} of 257 ng*h/mL. In some embodiments, the pharmaceutical composition provides an AUC_{Inf} of 258 ng*h/mL. In some embodiments, the pharmaceutical composition provides an AUC_{Inf} of 259 ng*h/mL. In some embodiments, the pharmaceutical composition provides an AUC_{Inf} of 260 ng*h/mL. In some embodiments, the pharmaceutical composition provides an AUC_{Inf} of 261 ng*h/mL. In some embodiments, the pharmaceutical composition provides an AUC_{Inf} of 262 ng*h/mL. In some embodiments, the pharmaceutical composition provides an AUC_{Inf} of 263 ng*h/mL. In some embodiments, the pharmaceutical composition provides an AUC_{Inf} of 264 ng*h/mL. In some embodiments, the pharmaceutical composition provides an AUC_{Inf} of 265 ng*h/mL. In some embodiments, the pharmaceutical composition provides an AUC_{Inf} of 266 ng*h/mL. In some embodiments, the pharmaceutical composition provides an AUC_{Inf} of 267 ng*h/mL. In some embodiments, the pharmaceutical composition provides an AUC_{Inf} of 268 ng*h/mL. In some embodiments, the pharmaceutical composition provides an AUC_{Inf} of 269 ng*h/mL. In some embodiments, the pharmaceutical composition provides an AUC_{Inf} of 270 ng*h/mL.

In some embodiments, the AUC_{Inf} is a geometric mean AUC_{Inf}.

In some embodiments, the range of AUC_{Inf} is a range of geometric mean AUC_{Inf}.

In some embodiments, the pharmaceutical composition provides a CL_{z}/F of between about 2 and about 12 L/h. In some embodiments, the pharmaceutical composition provides a CL_{z}/F of between about 3 and about 12 L/h. In some embodiments, the pharmaceutical composition provides a CL_{z}/F of between about 4 and about 12 L/h. In some embodiments, the pharmaceutical composition provides a CL_{z}/F of between about 5 and about 12 L/h. In some embodiments, the pharmaceutical composition provides a CL_{z}/F of between about 6 and about 12 L/h. In some embodiments, the pharmaceutical composition provides a CL_{z}/F of between about 7 and about 12 L/h. In some embodiments, the pharmaceutical composition provides a CL_{z}/F of between about 8 and about 12 L/h. In some embodiments, the pharmaceutical composition provides a CL_{z}/F of between about 9 and about 12 L/h. In some embodiments, the pharmaceutical composition provides a CL_{z}/F of between about 10 and about 12 L/h. In some embodiments, the pharmaceutical composition provides a CL_{z}/F of between about 11 and about 12 L/h. In some embodiments, the pharmaceutical composition provides a CL_{z}/F of between about 3 and about 7 L/h. In some embodiments, the pharmaceutical composition provides a CL_{z}/F of between about 4 and about 5 L/h.

In some embodiments, the pharmaceutical composition provides a CL_{z}/F of about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5 or about 12 L/h. In some embodiments, the pharmaceutical composition provides a CL_{z}/F of about 2 L/h. In some embodiments, the pharmaceutical composition provides a CL_{z}/F of about 2.5 L/h. In some embodiments, the pharmaceutical composition provides a CL_{z}/F of about 3 L/h. In some embodiments, the pharmaceutical composition provides a CL_{z}/F of about 3.5 L/h. In some embodiments, the pharmaceutical composition provides a CL_{z}/F of about 4 L/h. In some embodiments, the pharmaceutical composition provides a CL_{z}/F of about 4.5 L/h. In some embodiments, the pharmaceutical composition provides a CL_{z}/F of about 5 L/h. In some embodiments, the pharmaceutical composition provides a CL_{z}/F of about 5.5 L/h. In some embodiments, the pharmaceutical composition provides a CL_{z}/F of about 6 L/h. In some embodiments, the pharmaceutical composition provides a CL_{z}/F of about 6.5 L/h. In some embodiments, the pharmaceutical composition provides a CL_{z}/F of about 7 L/h. In some embodiments, the pharmaceutical composition provides a CL_{z}/F of about 7.5 L/h. In some embodiments, the pharmaceutical composition provides a CL_{z}/F of about 8 L/h. In some embodiments, the pharmaceutical composition provides a CL_{z}/F of about 8.5 L/h. In some embodiments, the pharmaceutical composition provides a CL_{z}/F of about 9 L/h. In some embodiments, the pharmaceutical composition provides a CL_{z}/F of about 9.5 L/h. In some embodiments, the pharmaceutical composition provides a CL_{z}/F of about 10 L/h. In some embodiments, the pharmaceutical composition provides a CL_{z}/F of about 10.5 L/h. In some embodiments, the pharmaceutical composition provides a CL_{z}/F of about 11 L/h. In some embodiments, the pharmaceutical composition provides a CL_{z}/F of about 11.5 L/h. In some embodiments, the pharmaceutical composition provides a CL_{z}/F of about 12 L/h.

In some embodiments, the pharmaceutical composition provides a CL_{z}/F of between 2 and 12 L/h. In some embodiments, the pharmaceutical composition provides a CL_{z}/F of between 3 and 12 L/h. In some embodiments, the pharmaceutical composition provides a CL_{z}/F of between 4 and 12 L/h. In some embodiments, the pharmaceutical composition provides a CL_{z}/F of between 5 and 12 L/h. In some embodiments, the pharmaceutical composition provides a CL_{z}/F of between 6 and 12 L/h. In some embodiments, the pharmaceutical composition provides a CL_{z}/F of between 7 and 12 L/h. In some embodiments, the pharmaceutical composition provides a CL_{z}/F of between 8 and 12 L/h. In some embodiments, the pharmaceutical composition provides a CL_{z}/F of between 9 and 12 L/h. In some embodiments, the pharmaceutical composition provides a CL_{z}/F of between 10 and 12 L/h. In some embodiments, the pharmaceutical composition provides a CL_{z}/F of between 11 and 12 L/h. In some embodiments, the pharmaceutical composition provides a CL_{z}/F of between 3 and 7 L/h. In some embodiments, the pharmaceutical composition provides a CL_{z}/F of between 4 and 5. L/h.

In some embodiments, the pharmaceutical composition provides a CL_{z}/F of 2, 2.5, 3, 3.5, 4, 4.5, 5, 5.5, 6, 6.5, 7, 7.5, 8, 8.5, 9, 9.5, 10, 10.5, 11, 11.5 or 12 L/h. In some embodiments, the pharmaceutical composition provides a CL_{z}/F of 2 L/h. In some embodiments, the pharmaceutical composition provides a CL_{z}/F of 2.5 L/h. In some embodiments, the pharmaceutical composition provides a CL_{z}/F of 3 L/h. In some embodiments, the pharmaceutical composition provides a CL_{z}/F of 3.5 L/h. In some embodiments, the pharmaceutical composition provides a CL_{z}/F of 4 L/h. In some embodiments, the pharmaceutical composition provides a CL_{z}/F of 4.5 L/h. In some embodiments, the pharmaceutical composition provides a CL_{z}/F of 5 L/h. In some embodiments, the pharmaceutical composition provides a CL_{z}/F of 5.5 L/h. In some embodiments, the pharmaceutical composition provides a CL_{z}/F of 6 L/h. In some embodiments, the pharmaceutical composition provides a CL_{z}/F of 6.5 L/h. In some embodiments, the pharmaceutical composition provides a CL_{z}/F of 7 L/h. In some embodiments, the pharmaceutical composition provides a CL_{z}/F of 7.5 L/h. In some embodiments, the pharmaceutical composition provides a CL_{z}/F of 8 L/h. In some embodiments, the pharmaceutical composition provides a CL_{z}/F of 8.5 L/h. In some embodiments, the pharmaceutical composition provides a CL_{z}/F of 9 L/h. In some embodiments, the pharmaceutical composition provides a CL_{z}/F of 9.5 L/h. In some embodiments, the pharmaceutical composition provides a CL_{z}/F of 10 L/h. In some embodiments, the pharmaceutical composition provides a CL_{z}/F of 10.5 L/h. In some embodiments, the pharmaceutical composition provides a CL_{z}/F of 11 L/h. In some embodiments, the pharmaceutical composition provides a CL_{z}/F of 11.5 L/h. In some embodiments, the pharmaceutical composition provides a CL_{z}/F of 12 L/h.

In some embodiments, the CL_{z}/F is a geometric mean CL_{z}/F.

In some embodiments, the range of CL_{z}/F is a range of geometric mean CL_{z}/F.

In some embodiments, the pharmaceutical composition provides a V_{z}/F of between about 100 and about 250 L. In some embodiments, the pharmaceutical composition provides a V_{z}/F of between about 125 and about 250 L. In some embodiments, the pharmaceutical composition provides a V_{z}/F of between about 150 and about 250 L. In some embodiments, the pharmaceutical composition provides a V_{z}/F of between about 175 and about 250 L. In some embodiments, the pharmaceutical composition provides a V_{z}/F of between about 200 and about 250 L. In some embodiments, the pharmaceutical composition provides a V_{z}/F of between about 130 and about 160 L. In some embodiments, the pharmaceutical composition provides a V_{z}/F of between about 140 and about 150 L.

In some embodiments, the pharmaceutical composition provides a V_{z}/F of about 100, about 110, about 120, about 130, about 140, about 150, about 160, about 170, about 180, about 190, about 200, about 210, about 220, about 230, about 240 or about 250 L. In some embodiments, the pharmaceutical composition provides a V_{z}/F of about 100 L. In some embodiments, the pharmaceutical composition provides a V_{z}/F of about 110 L. In some embodiments, the pharmaceutical composition provides a V_{z}/F of about 120 L. In some embodiments, the pharmaceutical composition provides a V_{z}/F of about 130 L. In some embodiments, the pharmaceutical composition provides a V_{z}/F of about 140 L. In some embodiments, the pharmaceutical composition provides a V_{z}/F of about 150 L. In some embodiments, the pharmaceutical composition provides a V_{z}/F of about 160 L. In some embodiments, the pharmaceutical composition provides a V_{z}/F of about 170 L. In some embodiments, the pharmaceutical composition provides a V_{z}/F of about 180 L. In some embodiments, the pharmaceutical composition provides a V_{z}/F of about 190 L. In some embodiments, the pharmaceutical composition provides a V_{z}/F of about 200 L. In some embodiments, the pharmaceutical composition provides a V_{z}/F of about 210 L. In some embodiments, the pharmaceutical composition provides a V_{z}/F of about 220 L. In some embodiments, the pharmaceutical composition provides a V_{z}/F of about 230 L. In some embodiments, the pharmaceutical composition provides a V_{z}/F of about 240 L. In some embodiments, the pharmaceutical composition provides a V_{z}/F of about 250 L.

In some embodiments, the pharmaceutical composition provides a V_{z}/F of between 100 and 250 L. In some embodiments, the pharmaceutical composition provides a V_{z}/F of between 125 and 250 L. In some embodiments, the pharmaceutical composition provides a V_{z}/F of between 150 and 250 L. In some embodiments, the pharmaceutical composition provides a V_{z}/F of between 175 and 250 L. In some embodiments, the pharmaceutical composition provides a V_{z}/F of between 200 and 250 L. In some embodiments, the pharmaceutical composition provides a V_{z}/F of between 130 and 160 L. In some embodiments, the pharmaceutical composition provides a V_{z}/F of between 140 and 150 L.

In some embodiments, the pharmaceutical composition provides a V_{z}/F of 100, 110, 120, 130, 140, 150, 160, 170, 180, 190, 200, 210, 220, 230, 240 or 250 L. In some embodiments, the pharmaceutical composition provides a V_{z}/F of 100 L. In some embodiments, the pharmaceutical composition provides a V_{z}/F of 110 L. In some embodiments, the pharmaceutical composition provides a V_{z}/F of 120 L. In some embodiments, the pharmaceutical composition provides a V_{z}/F of 130 L. In some embodiments, the pharmaceutical composition provides a V_{z}/F of 140 L. In some embodiments, the pharmaceutical composition provides a V_{z}/F of 150 L. In some embodiments, the pharmaceutical composition provides a V_{z}/F of 160 L. In some embodiments, the pharmaceutical composition provides a V_{z}/F of 170 L. In some embodiments, the pharmaceutical composition provides a V_{z}/F of 180 L. In some embodiments, the pharmaceutical composition provides a V_{z}/F of 190 L. In some embodiments, the pharmaceutical composition provides a V_{z}/F of 200 L. In some embodiments, the pharmaceutical composition provides a V_{z}/F of 210 L. In some embodiments, the pharmaceutical composition provides a V_{z}/F of 220 L. In some embodiments, the pharmaceutical composition provides a V_{z}/F of 230 L. In some embodiments, the pharmaceutical composition provides a V_{z}/F of 240 L. In some embodiments, the pharmaceutical composition provides a V_{z}/F of 250 L.

In some embodiments, the V_{z}/F is a geometric mean CL_{z}/F.

In some embodiments, the range of V_{z}/F is a range of geometric mean V_{z}/F.

In some embodiments, the pharmaceutical composition provides a bioavailability of about 70 to about 100%. In some embodiments, the pharmaceutical composition provides a bioavailability of about 80 to about 100%. In some embodiments, the pharmaceutical composition provides a bioavailability of about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99 or about 100%. In some embodiments, the pharmaceutical composition provides a bioavailability of about 85 to about 100%. In some embodiments, the pharmaceutical composition provides a bioavailability of about 90 to about 100%. In some embodiments, the pharmaceutical composition provides a bioavailability of about 95 to about 100%. In some embodiments, the pharmaceutical composition provides a bioavailability of about 100%.

In some embodiments, the pharmaceutical composition provides a bioavailability of 70 to 100%. In some embodiments, the pharmaceutical composition provides a bioavailability of 80 to 100%. In some embodiments, the pharmaceutical composition provides a bioavailability of 90 to 100%. In some embodiments, the pharmaceutical composition provides a bioavailability of 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. In some embodiments, the pharmaceutical composition provides a bioavailability of 85 to 100%. In some embodiments, the pharmaceutical composition provides a bioavailability of 90 to 100%. In some embodiments, the pharmaceutical composition provides a bioavailability of 95 to 100%. In some embodiments, the pharmaceutical composition provides a bioavailability of 100%.

In some embodiments, the pharmaceutical composition provides a Cₘₐₓ of between about 8 and about 25 ng/mL; an AUCₗₐₛₜ of between about 100 and about 450 ng*h/mL; and a tₗₐₛₜ of between 20 and 200 hours.

In some embodiments, the pharmaceutical composition provides a Cₘₐₓ of between about 8 and about 25 ng/mL; an AUCₗₐₛₜ of between about 100 and about 450 ng*h/mL; a tₗₐₛₜ of between 20 and 200 hours; and a tₘₐₓ of between about 2 and about 8 hours.

In some embodiments, the pharmaceutical composition provides a Cₘₐₓ of between about 8 and about 25 ng/mL; an AUCₗₐₛₜ of between about 100 and about 450 ng*h/mL; a tₗₐₛₜ of between 20 and 200 hours; and a V_{z}/F of between about 100 and about 250 L.

In some embodiments, the pharmaceutical composition provides a Cₘₐₓ of between about 8 and about 25 ng/mL; an AUCₗₐₛₜ of between about 100 and about 450 ng*h/mL; a tₗₐₛₜ of between 20 and 200 hours; and a CL_{z}/F of between about 2 and about 12 L/h.

In some embodiments, the pharmaceutical composition provides a Cₘₐₓ of between about 8 and about 25 ng/mL; an AUCₗₐₛₜ of between about 100 and about 450 ng*h/mL; a tₗₐₛₜ of between 20 and 200 hours; and a bioavailability of between about 70 and about 100%.

In some embodiments, the pharmaceutical composition provides a Cₘₐₓ of between about 8 and about 25 ng/mL; an AUCₗₐₛₜ of between about 100 and about 450 ng*h/mL; a tₗₐₛₜ of between 20 and 200 hours; a tₘₐₓ of between about 2 and about 8 hours; and a V_{z}/F of between about 100 and about 250 L.

In some embodiments, the pharmaceutical composition provides a Cₘₐₓ of between about 8 and about 25 ng/mL; an AUCₗₐₛₜ of between about 100 and about 450 ng*h/mL; a tₗₐₛₜ of between 20 and 200 hours; a tₘₐₓ of between about 2 and about 8 hours; a CL_{z}/F of between about 2 and about 12 L/h.

In some embodiments, the pharmaceutical composition provides a Cₘₐₓ of between about 8 and about 25 ng/mL; an AUCₗₐₛₜ of between about 100 and about 450 ng*h/mL; a tₗₐₛₜ of between 20 and 200 hours; a tₘₐₓ of between about 2 and about 8 hours; a V_{z}/F of between about 100 and about 250 L; and a CL_{z}/F of between about 2 and about 12 L/h.

In some embodiments, the pharmaceutical composition provides a Cₘₐₓ of between about 10 and about 18 ng/mL; an AUCₗₐₛₜ of between about 150 and about 250 ng*h/mL; and a tₗₐₛₜ of between 50 and 70 hours.

In some embodiments, the pharmaceutical composition provides a Cₘₐₓ of between about 10 and about 18 ng/mL; an AUCₗₐₛₜ of between about 150 and about 250 ng*h/mL; a tₗₐₛₜ of between 50 and 70 hours; and a tₘₐₓ of between about 3 and about 5 hours.

In some embodiments, the pharmaceutical composition provides a Cₘₐₓ of between about 10 and about 18 ng/mL; an AUCₗₐₛₜ of between about 150 and about 250 ng*h/mL; a tₗₐₛₜ of between 50 and 70 hours; and a V_{z}/F of between about 140 and about 160 L.

In some embodiments, the pharmaceutical composition provides a Cₘₐₓ of between about 10 and about 18 ng/mL; an AUCₗₐₛₜ of between about 150 and about 250 ng*h/mL; a tₗₐₛₜ of between 50 and 70 hours; and a CL_{z}/F of between about 3 and about 6 L/h.

In some embodiments, the pharmaceutical composition provides a Cₘₐₓ of between about 10 and about 18 ng/mL; an AUCₗₐₛₜ of between about 150 and about 250 ng*h/mL; a tₗₐₛₜ of between 50 and 70 hours; and a bioavailability of between about 70 and about 100%.

In some embodiments, the pharmaceutical composition provides a Cₘₐₓ of between about 10 and about 18 ng/mL; an AUCₗₐₛₜ of between about 150 and about 250 ng*h/mL; a tₗₐₛₜ of between 50 and 70 hours; a tₘₐₓ of between about 2 and about 8 hours; and a V_{z}/F of between about 140 and about 160 L.

In some embodiments, the pharmaceutical composition provides a Cₘₐₓ of between about 10 and about 18 ng/mL; an AUCₗₐₛₜ of between about 150 and about 250 ng*h/mL; a tₗₐₛₜ of between 70 and 700 hours; a tₘₐₓ of between about 2 and about 8 hours; a CL_{z}/F of between about 3 and about 5 L/h.

In some embodiments, the pharmaceutical composition provides a Cₘₐₓ of between about 10 and about 18 ng/mL; an AUCₗₐₛₜ of between about 150 and about 250 ng*h/mL; a tₗₐₛₜ of between 50 and 70 hours; a tₘₐₓ of between about 2 and about 8 hours; a V_{z}/F of between about 140 and about 160 L; and a CL_{z}/F of between about 3 and about 5 L/h.

In some embodiments, the pharmaceutical composition provides a Cₘₐₓ of between about 15 and about 18 ng/mL; an AUCₗₐₛₜ of between about 200 and about 250 ng*h/mL; and a tₗₐₛₜ of between 50 and 70 hours.

In some embodiments, the pharmaceutical composition provides a Cₘₐₓ of between about 15 and about 18 ng/mL; an AUCₗₐₛₜ of between about 200 and about 250 ng*h/mL; a tₗₐₛₜ of between 50 and 70 hours; and a tₘₐₓ of between about 3 and about 5 hours.

In some embodiments, the pharmaceutical composition provides a Cₘₐₓ of between about 15 and about 18 ng/mL; an AUCₗₐₛₜ of between about 200 and about 250 ng*h/mL; a tₗₐₛₜ of between 50 and 70 hours; and a V_{z}/F of between about 140 and about 160 L.

In some embodiments, the pharmaceutical composition provides a Cₘₐₓ of between about 15 and about 18 ng/mL; an AUCₗₐₛₜ of between about 200 and about 250 ng*h/mL; a tₗₐₛₜ of between 50 and 70 hours; and a CL_{z}/F of between about 3 and about 6 L/h.

In some embodiments, the pharmaceutical composition provides a Cₘₐₓ of between about 15 and about 18 ng/mL; an AUCₗₐₛₜ of between about 200 and about 250 ng*h/mL; a tₗₐₛₜ of between 50 and 70 hours; and a bioavailability of between about 70 and about 100%.

In some embodiments, the pharmaceutical composition provides a Cₘₐₓ of between about 15 and about 18 ng/mL; an AUCₗₐₛₜ of between about 200 and about 250 ng*h/mL; a tₗₐₛₜ of between 50 and 70 hours; a tₘₐₓ of between about 2 and about 8 hours; and a V_{z}/F of between about 140 and about 160 L.

In some embodiments, the pharmaceutical composition provides a Cₘₐₓ of between about 15 and about 18 ng/mL; an AUCₗₐₛₜ of between about 200 and about 250 ng*h/mL; a tₗₐₛₜ of between 70 and 700 hours; a tₘₐₓ of between about 2 and about 8 hours; a CL_{z}/F of between about 3 and about 5 L/h.

In some embodiments, the pharmaceutical composition provides a Cₘₐₓ of between about 15 and about 18 ng/mL; an AUCₗₐₛₜ of between about 200 and about 250 ng*h/mL; a tₗₐₛₜ of between 50 and 70 hours; a tₘₐₓ of between about 2 and about 8 hours; a V_{z}/F of between about 140 and about 160 L; and a CL_{z}/F of between about 3 and about 5 L/h.

In some embodiments, the pharmaceutical composition provides a Cₘₐₓ of between about 8 and about 25 ng/mL; and an AUC_{inf} of between about 100 and about 500 ng*h/mL.

In some embodiments, the pharmaceutical composition provides a Cₘₐₓ of between about 8 and about 25 ng/mL; an AUC_{inf} of between about 100 and about 500 ng*h/mL; and a tₘₐₓ of between about 2 and about 8 hours.

In some embodiments, the pharmaceutical composition provides a Cₘₐₓ of between about 8 and about 25 ng/mL; an AUC_{inf} of between about 100 and about 500 ng*h/mL; and a V_{z}/F of between about 100 and about 250 L.

In some embodiments, the pharmaceutical composition provides a Cₘₐₓ of between about 8 and about 25 ng/mL; an AUC_{inf} of between about 100 and about 500 ng*h/mL; and a CL_{z}/F of between about 2 and about 12 L/h.

In some embodiments, the pharmaceutical composition provides a Cₘₐₓ of between about 8 and about 25 ng/mL; an AUC_{inf} of between about 100 and about 500 ng*h/mL; and a bioavailability of between about 70 and about 100%.

In some embodiments, the pharmaceutical composition provides a Cₘₐₓ of between about 8 and about 25 ng/mL; an AUC_{inf} of between about 100 and about 500 ng*h/mL; a tₘₐₓ of between about 2 and about 8 hours; and a V_{z}/F of between about 100 and about 250 L.

In some embodiments, the pharmaceutical composition provides a Cₘₐₓ of between about 8 and about 25 ng/mL; an AUC_{inf} of between about 100 and about 500 ng*h/mL; a tₘₐₓ of between about 2 and about 8 hours; a CL_{z}/F of between about 2 and about 12 L/h.

In some embodiments, the pharmaceutical composition provides a Cₘₐₓ of between about 8 and about 25 ng/mL; an AUC_{inf} of between about 100 and about 500 ng*h/mL; a tₘₐₓ of between about 2 and about 8 hours; a V_{z}/F of between about 100 and about 250 L; and a CL_{z}/F of between about 2 and about 12 L/h.

In some embodiments, the pharmaceutical composition provides a Cₘₐₓ of between about 10 and about 18 ng/mL; an AUC_{inf} of between about 150 and about 300 ng*h/mL; and a tₗₐₛₜ of between 50 and 70 hours.

In some embodiments, the pharmaceutical composition provides a Cₘₐₓ of between about 10 and about 18 ng/mL; an AUC_{inf} of between about 150 and about 300 ng*h/mL; and a tₘₐₓ of between about 3 and about 5 hours.

In some embodiments, the pharmaceutical composition provides a Cₘₐₓ of between about 10 and about 18 ng/mL; an AUC_{inf} of between about 150 and about 300 ng*h/mL; and a V_{z}/F of between about 140 and about 160 L.

In some embodiments, the pharmaceutical composition provides a Cₘₐₓ of between about 10 and about 18 ng/mL; an AUC_{inf} of between about 150 and about 300 ng*h/mL; and a CL_{z}/F of between about 3 and about 6 L/h.

In some embodiments, the pharmaceutical composition provides a Cₘₐₓ of between about 10 and about 18 ng/mL; an AUC_{inf} of between about 150 and about 300 ng*h/mL; and a bioavailability of between about 70 and about 100%.

In some embodiments, the pharmaceutical composition provides a Cₘₐₓ of between about 10 and about 18 ng/mL; an AUC_{inf} of between about 150 and about 300 ng*h/mL; a tₘₐₓ of between about 3 and about 5 hours; and a V_{z}/F of between about 140 and about 160 L.

In some embodiments, the pharmaceutical composition provides a Cₘₐₓ of between about 10 and about 18 ng/mL; an AUC_{inf} of between about 150 and about 300 ng*h/mL; a tₘₐₓ of between about 3 and about 5 hours; a CL_{z}/F of between about 3 and about 6 L/h.

In some embodiments, the pharmaceutical composition provides a Cₘₐₓ of between about 10 and about 18 ng/mL; an AUC_{inf} of between about 150 and about 300 ng*h/mL; a tₘₐₓ of between about 3 and about 5 hours; a V_{z}/F of between about 140 and about 160 L; and a CL_{z}/F of between about 3 and about 6 L/h.

In some embodiments, the pharmaceutical composition provides a Cₘₐₓ of between about 15 and about 18 ng/mL; an AUC_{inf} of between about 200 and about 300 ng*h/mL; and a tₗₐₛₜ of between 50 and 70 hours.

In some embodiments, the pharmaceutical composition provides a Cₘₐₓ of between about 15 and about 18 ng/mL; an AUC_{inf} of between about 200 and about 300 ng*h/mL; and a tₘₐₓ of between about 3 and about 5 hours.

In some embodiments, the pharmaceutical composition provides a Cₘₐₓ of between about 15 and about 18 ng/mL; an AUC_{inf} of between about 200 and about 300 ng*h/mL; and a V_{z}/F of between about 140 and about 160 L.

In some embodiments, the pharmaceutical composition provides a Cₘₐₓ of between about 15 and about 18 ng/mL; an AUC_{inf} of between about 200 and about 300 ng*h/mL; and a CL_{z}/F of between about 3 and about 6 L/h.

In some embodiments, the pharmaceutical composition provides a Cₘₐₓ of between about 15 and about 18 ng/mL; an AUC_{inf} of between about 200 and about 300 ng*h/mL; and a bioavailability of between about 70 and about 100%.

In some embodiments, the pharmaceutical composition provides a Cₘₐₓ of between about 15 and about 18 ng/mL; an AUC_{inf} of between about 200 and about 300 ng*h/mL; a tₘₐₓ of between about 3 and about 5 hours; and a V_{z}/F of between about 140 and about 160 L.

In some embodiments, the pharmaceutical composition provides a Cₘₐₓ of between about 15 and about 18 ng/mL; an AUC_{inf} of between about 200 and about 300 ng*h/mL; a tₘₐₓ of between about 3 and about 5 hours; a CL_{z}/F of between about 3 and about 6 L/h.

In some embodiments, the pharmaceutical composition provides a Cₘₐₓ of between about 15 and about 18 ng/mL; an AUC_{inf} of between about 200 and about 300 ng*h/mL; a tₘₐₓ of between about 3 and about 5 hours; a V_{z}/F of between about 140 and about 160 L; and a CL_{z}/F of between about 3 and about 6 L/h.

In some embodiments, the pharmaceutical composition provides a Cₘₐₓ of between 8 and 25 ng/mL; an AUCₗₐₛₜ of between 100 and 450 ng*h/mL; and a tₗₐₛₜ of between 20 and 200 hours.

In some embodiments, the pharmaceutical composition provides a Cₘₐₓ of between 8 and 25 ng/mL; an AUCₗₐₛₜ of between 100 and 450 ng*h/mL; a tₗₐₛₜ of between 20 and 200 hours; and a tₘₐₓ of between 2 and 8 hours.

In some embodiments, the pharmaceutical composition provides a Cₘₐₓ of between 8 and 25 ng/mL; an AUCₗₐₛₜ of between 100 and 450 ng*h/mL; a tₗₐₛₜ of between 20 and 200 hours; and a V_{z}/F of between 100 and 250 L.

In some embodiments, the pharmaceutical composition provides a Cₘₐₓ of between 8 and 25 ng/mL; an AUCₗₐₛₜ of between 100 and 450 ng*h/mL; a tₗₐₛₜ of between 20 and 200 hours; and a CL_{z}/F of between 2 and 12 L/h.

In some embodiments, the pharmaceutical composition provides a Cₘₐₓ of between 8 and 25 ng/mL; an AUCₗₐₛₜ of between 100 and 450 ng*h/mL; a tₗₐₛₜ of between 20 and 200 hours; and a bioavailability of between 70 and 100%.

In some embodiments, the pharmaceutical composition provides a Cₘₐₓ of between 8 and 25 ng/mL; an AUCₗₐₛₜ of between 100 and 450 ng*h/mL; a tₗₐₛₜ of between 20 and 200 hours; a tₘₐₓ of between 2 and 8 hours; and a V_{z}/F of between 100 and 250 L.

In some embodiments, the pharmaceutical composition provides a Cₘₐₓ of between 8 and 25 ng/mL; an AUCₗₐₛₜ of between 100 and 450 ng*h/mL; a tₗₐₛₜ of between 20 and 200 hours; a tₘₐₓ of between 2 and 8 hours; a CL_{z}/F of between 2 and 12 L/h.

In some embodiments, the pharmaceutical composition provides a Cₘₐₓ of between 8 and 25 ng/mL; an AUCₗₐₛₜ of between 100 and 450 ng*h/mL; a tₗₐₛₜ of between 20 and 200 hours; a tₘₐₓ of between 2 and 8 hours; a V_{z}/F of between 100 and 250 L; and a CL_{z}/F of between 2 and 12 L/h.

In some embodiments, the pharmaceutical composition provides a Cₘₐₓ of between 10 and 18 ng/mL; an AUCₗₐₛₜ of between 150 and 250 ng*h/mL; and a tₗₐₛₜ of between 50 and 70 hours.

In some embodiments, the pharmaceutical composition provides a Cₘₐₓ of between 10 and 18 ng/mL; an AUCₗₐₛₜ of between 150 and 250 ng*h/mL; a tₗₐₛₜ of between 50 and 70 hours; and a tₘₐₓ of between 3 and 5 hours.

In some embodiments, the pharmaceutical composition provides a Cₘₐₓ of between 10 and 18 ng/mL; an AUCₗₐₛₜ of between 150 and 250 ng*h/mL; a tₗₐₛₜ of between 50 and 70 hours; and a V_{z}/F of between 140 and 160 L.

In some embodiments, the pharmaceutical composition provides a Cₘₐₓ of between 10 and 18 ng/mL; an AUCₗₐₛₜ of between 150 and 250 ng*h/mL; a tₗₐₛₜ of between 50 and 70 hours; and a CL_{z}/F of between 3 and 6 L/h.

In some embodiments, the pharmaceutical composition provides a Cₘₐₓ of between 10 and 18 ng/mL; an AUCₗₐₛₜ of between 150 and 250 ng*h/mL; a tₗₐₛₜ of between 50 and 70 hours; and a bioavailability of between 70 and 100%.

In some embodiments, the pharmaceutical composition provides a Cₘₐₓ of between 10 and 18 ng/mL; an AUCₗₐₛₜ of between 150 and 250 ng*h/mL; a tₗₐₛₜ of between 50 and 70 hours; a tₘₐₓ of between 2 and 8 hours; and a V_{z}/F of between 140 and 160 L.

In some embodiments, the pharmaceutical composition provides a Cₘₐₓ of between 10 and 18 ng/mL; an AUCₗₐₛₜ of between 150 and 250 ng*h/mL; a tₗₐₛₜ of between 70 and 700 hours; a tₘₐₓ of between 2 and 8 hours; a CL_{z}/F of between 3 and 5 L/h.

In some embodiments, the pharmaceutical composition provides a Cₘₐₓ of between 10 and 18 ng/mL; an AUCₗₐₛₜ of between 150 and 250 ng*h/mL; a tₗₐₛₜ of between 50 and 70 hours; a tₘₐₓ of between 2 and 8 hours; a V_{z}/F of between 140 and 160 L; and a CL_{z}/F of between 3 and 5 L/h.

In some embodiments, the pharmaceutical composition provides a Cₘₐₓ of between 15 and 18 ng/mL; an AUCₗₐₛₜ of between 200 and 250 ng*h/mL; and a tₗₐₛₜ of between 50 and 70 hours.

In some embodiments, the pharmaceutical composition provides a Cₘₐₓ of between 15 and 18 ng/mL; an AUCₗₐₛₜ of between 200 and 250 ng*h/mL; a tₗₐₛₜ of between 50 and 70 hours; and a tₘₐₓ of between 3 and 5 hours.

In some embodiments, the pharmaceutical composition provides a Cₘₐₓ of between 15 and 18 ng/mL; an AUCₗₐₛₜ of between 200 and 250 ng*h/mL; a tₗₐₛₜ of between 50 and 70 hours; and a V_{z}/F of between 140 and 160 L.

In some embodiments, the pharmaceutical composition provides a Cₘₐₓ of between 15 and 18 ng/mL; an AUCₗₐₛₜ of between 200 and 250 ng*h/mL; a tₗₐₛₜ of between 50 and 70 hours; and a CL_{z}/F of between 3 and 6 L/h.

In some embodiments, the pharmaceutical composition provides a Cₘₐₓ of between 15 and 18 ng/mL; an AUCₗₐₛₜ of between 200 and 250 ng*h/mL; a tₗₐₛₜ of between 50 and 70 hours; and a bioavailability of between 70 and 100%.

In some embodiments, the pharmaceutical composition provides a Cₘₐₓ of between 15 and 18 ng/mL; an AUCₗₐₛₜ of between 200 and 250 ng*h/mL; a tₗₐₛₜ of between 50 and 70 hours; a tₘₐₓ of between 2 and 8 hours; and a V_{z}/F of between 140 and 160 L.

In some embodiments, the pharmaceutical composition provides a Cₘₐₓ of between 15 and 18 ng/mL; an AUCₗₐₛₜ of between 200 and 250 ng*h/mL; a tₗₐₛₜ of between 70 and 700 hours; a tₘₐₓ of between 2 and 8 hours; a CL_{z}/F of between 3 and 5 L/h.

In some embodiments, the pharmaceutical composition provides a Cₘₐₓ of between 15 and 18 ng/mL; an AUCₗₐₛₜ of between 200 and 250 ng*h/mL; a tₗₐₛₜ of between 50 and 70 hours; a tₘₐₓ of between 2 and 8 hours; a V_{z}/F of between 140 and 160 L; and a CL_{z}/F of between 3 and 5 L/h.

In some embodiments, the pharmaceutical composition provides a Cₘₐₓ of between 8 and 25 ng/mL; and an AUC_{inf} of between 100 and 500 ng*h/mL.

In some embodiments, the pharmaceutical composition provides a Cₘₐₓ of between 8 and 25 ng/mL; an AUC_{inf} of between 100 and 500 ng*h/mL; and a tₘₐₓ of between 2 and 8 hours.

In some embodiments, the pharmaceutical composition provides a Cₘₐₓ of between 8 and 25 ng/mL; an AUC_{inf} of between 100 and 500 ng*h/mL; and a V_{z}/F of between 100 and 250 L.

In some embodiments, the pharmaceutical composition provides a Cₘₐₓ of between 8 and 25 ng/mL; an AUC_{inf} of between 100 and 500 ng*h/mL; and a CL_{z}/F of between 2 and 12 L/h.

In some embodiments, the pharmaceutical composition provides a Cₘₐₓ of between 8 and 25 ng/mL; an AUC_{inf} of between 100 and 500 ng*h/mL; and a bioavailability of between 70 and 100%.

In some embodiments, the pharmaceutical composition provides a Cₘₐₓ of between 8 and 25 ng/mL; an AUC_{inf} of between 100 and 500 ng*h/mL; a tₘₐₓ of between 2 and 8 hours; and a V_{z}/F of between 100 and 250 L.

In some embodiments, the pharmaceutical composition provides a Cₘₐₓ of between 8 and 25 ng/mL; an AUC_{inf} of between 100 and 500 ng*h/mL; a tₘₐₓ of between 2 and 8 hours; a CL_{z}/F of between 2 and 12 L/h.

In some embodiments, the pharmaceutical composition provides a Cₘₐₓ of between 8 and 25 ng/mL; an AUC_{inf} of between 100 and 500 ng*h/mL; a tₘₐₓ of between 2 and 8 hours; a V_{z}/F of between 100 and 250 L; and a CL_{z}/F of between 2 and 12 L/h.

In some embodiments, the pharmaceutical composition provides a Cₘₐₓ of between 10 and 18 ng/mL; an AUC_{inf} of between 150 and 300 ng*h/mL; and a tₗₐₛₜ of between 50 and 70 hours.

In some embodiments, the pharmaceutical composition provides a Cₘₐₓ of between 10 and 18 ng/mL; an AUC_{inf} of between 150 and 300 ng*h/mL; and a tₘₐₓ of between 3 and 5 hours.

In some embodiments, the pharmaceutical composition provides a Cₘₐₓ of between 10 and 18 ng/mL; an AUC_{inf} of between 150 and 300 ng*h/mL; and a V_{z}/F of between 140 and 160 L.

In some embodiments, the pharmaceutical composition provides a Cₘₐₓ of between 10 and 18 ng/mL; an AUC_{inf} of between 150 and 300 ng*h/mL; and a CL_{z}/F of between 3 and 6 L/h.

In some embodiments, the pharmaceutical composition provides a Cₘₐₓ of between 10 and 18 ng/mL; an AUC_{inf} of between 150 and 300 ng*h/mL; and a bioavailability of between 70 and 100%.

In some embodiments, the pharmaceutical composition provides a Cₘₐₓ of between 10 and 18 ng/mL; an AUC_{inf} of between 150 and 300 ng*h/mL; a tₘₐₓ of between 3 and 5 hours; and a V_{z}/F of between 140 and 160 L.

In some embodiments, the pharmaceutical composition provides a Cₘₐₓ of between 10 and 18 ng/mL; an AUC_{inf} of between 150 and 300 ng*h/mL; a tₘₐₓ of between 3 and 5 hours; a CL_{z}/F of between 3 and 6 L/h.

In some embodiments, the pharmaceutical composition provides a Cₘₐₓ of between 10 and 18 ng/mL; an AUC_{inf} of between 150 and 300 ng*h/mL; a tₘₐₓ of between 3 and 5 hours; a V_{z}/F of between 140 and 160 L; and a CL_{z}/F of between 3 and 6 L/h.

In some embodiments, the pharmaceutical composition provides a Cₘₐₓ of between 15 and 18 ng/mL; an AUC_{inf} of between 200 and 300 ng*h/mL; and a tₗₐₛₜ of between 50 and 70 hours.

In some embodiments, the pharmaceutical composition provides a Cₘₐₓ of between 15 and 18 ng/mL; an AUC_{inf} of between 200 and 300 ng*h/mL; and a tₘₐₓ of between 3 and 5 hours.

In some embodiments, the pharmaceutical composition provides a Cₘₐₓ of between 15 and 18 ng/mL; an AUC_{inf} of between 200 and 300 ng*h/mL; and a V_{z}/F of between 140 and 160 L.

In some embodiments, the pharmaceutical composition provides a Cₘₐₓ of between 15 and 18 ng/mL; an AUC_{inf} of between 200 and 300 ng*h/mL; and a CL_{z}/F of between 3 and 6 L/h.

In some embodiments, the pharmaceutical composition provides a Cₘₐₓ of between 15 and 18 ng/mL; an AUC_{inf} of between 200 and 300 ng*h/mL; and a bioavailability of between 70 and 100%.

In some embodiments, the pharmaceutical composition provides a Cₘₐₓ of between 15 and 18 ng/mL; an AUC_{inf} of between 200 and 300 ng*h/mL; a tₘₐₓ of between 3 and 5 hours; and a V_{z}/F of between 140 and 160 L.

In some embodiments, the pharmaceutical composition provides a Cₘₐₓ of between 15 and 18 ng/mL; an AUC_{inf} of between 200 and 300 ng*h/mL; a tₘₐₓ of between 3 and 5 hours; a CL_{z}/F of between 3 and 6 L/h.

In some embodiments, the pharmaceutical composition provides a Cₘₐₓ of between 15 and 18 ng/mL; an AUC_{inf} of between 200 and 300 ng*h/mL; a tₘₐₓ of between 3 and 5 hours; a V_{z}/F of between 140 and 160 L; and a CL_{z}/F of between 3 and 6 L/h.

In some embodiments, the pharmaceutical composition of the present disclosure exhibits a PK profile following administration to a subject in a fasted state that is substantially similar to that following administration to a subject in a fed/post meal state. In some embodiments, the pharmaceutical compositions of the present disclosure may exhibit a PK profile consistent with that desired for once-daily dosing. In some embodiments, the pharmaceutical compositions exhibit a PK profile consistent with that desired for administration of a loading dose followed by a daily maintenance dose.

In some embodiments, the pharmaceutical composition that provides the PK parameters disclosed herein may be administered by a variety of routes including, oral, parenteral, rectal, transdermal, intradermal, intrathecal, subcutaneous, intravenous, intramuscular, and intranasal. In some embodiments, the pharmaceutical composition that provides the PK parameters disclosed herein may be in any dosage form (e.g., solid (e.g., pills, tablets, capsules, or the like), liquid, lipid-based (e.g., gelatin capsules), prefilled, premeasured ampules or syringes of the liquid compositions).

In some embodiments, the pharmaceutical composition that provides the PK parameters comprises Compound I or a pharmaceutically acceptable salt thereof, and one or more glycerides, wherein said glycerides comprise about 30% to about 100% w/w monoglycerides.

### Methods of Use

Compound I as described herein, is a positive allosteric modulator of NMDA receptor function, and therefore is useful for the treatment and prevention of, e.g., certain CNS-related conditions in a subject.

In one aspect, the disclosure provides a method for treating a disease, disorder or condition requiring positive allosteric NMDA receptor modulation in a subject, comprising administering to the subject an effective amount of a pharmaceutical composition as disclosed herein.

In some embodiments, the disclosure provides a method for treating a CNS-related condition in a subject, comprising administering to the subject an effective amount of a pharmaceutical composition as disclosed herein.

In some embodiments, the disclosure provides a method for preventing a disease, disorder or condition requiring positive allosteric NMDA receptor modulation in a subject, comprising administering to the subject an effective amount of a pharmaceutical composition as disclosed herein.

In some embodiments, the disclosure provides a pharmaceutical composition as disclosed herein, for use in treating a disease, disorder or condition requiring positive NMDA receptor modulation in a subject.

In some embodiments, the disclosure provides a pharmaceutical composition as disclosed herein, for use in preventing a disease, disorder or condition requiring positive NMDA receptor modulation in a subject.

In some embodiments, the disclosure provides the use of a pharmaceutical composition as disclosed herein for the manufacture of a medicament for treating a disease, disorder or condition requiring positive allosteric NMDA receptor modulation in a subject.

In some embodiments, the disclosure provides the use of a pharmaceutical composition as disclosed herein for the manufacture of a medicament for preventing a disease, disorder or condition requiring positive allosteric NMDA receptor modulation in a subject.

In some embodiments, the disclosure provides a method for treating a disease, disorder or condition requiring positive allosteric NMDA receptor modulation in a subject, wherein following administration of the pharmaceutical composition to the subject, the pharmaceutical composition provides one or more of the following pharmacokinetic parameters: a) a Cₘₐₓ of between about 8 and about 25 ng/mL; b) an AUCₗₐₛₜ of between about 100 and about 450 ng*h/mL; c) an AUC_{inf} of between about 100 and about 500 ng*h/mL; d) a tₘₐₓ of between about 2 and about 8 hours; e) a V_{z}/F of between about 100 and about 250 L; f) a CL_{z}/F of between about 2 and about 12 L/h; or g) a bioavailability of between about 70 and about 100%.

In some embodiments, the CNS-related condition is selected from the group consisting of Huntington's disease, Parkinson's disease and Alzheimer's disease.

### Parkinson's Disease

Parkinson's disease affects nerve cells in the brain that produce dopamine. Symptoms include muscle rigidity, tremors, and changes in speech and gait. Parkinsonism is characterized by tremor, bradykinesia, rigidity, and postural instability. Parkinsonism shares symptoms found in Parkinson's disease but is a symptom complex rather than a progressive neurodegenerative disease.

In one aspect, the disclosure provides a method of treating or preventing Parkinson's disease in a subject, comprising administering to the subject a therapeutically effective amount of a pharmaceutical composition as described herein.

In some embodiments, the disclosure provides a method of treating Parkinson's disease in a subject, comprising administering to the subject a therapeutically effective amount of a pharmaceutical composition as described herein. In some embodiments, the disclosure provides a method of preventing Parkinson's disease in a subject, comprising administering to the subject a therapeutically effective amount of a pharmaceutical composition as described herein.

In some embodiments, the disclosure provides a method of treating cognitive impairment in a subject having Parkinson's disease, comprising administering to the subject a therapeutically effective amount of a pharmaceutical composition as described herein.

In some embodiments, the disclosure provides a method of treating Mild Cognitive Impairment (MCI) associated with Parkinson's disease in a subject, comprising administering to the subject a therapeutically effective amount of a pharmaceutical composition as described herein.

In some embodiments, the disclosure provides a method of improving executive function in a subject having Mild Cognitive Impairment (MCI) associated with Parkinson's disease, comprising administering to the subject a therapeutically effective amount of a pharmaceutical composition as described herein.

In some embodiments, the disclosure provides a method of improving working memory in a subject having Mild Cognitive Impairment (MCI) associated with Parkinson's disease, comprising administering to the subject a therapeutically effective amount of a pharmaceutical composition as described herein.

In some embodiments, the disclosure provides a method of improving learning in a subject having Mild Cognitive Impairment (MCI) associated with Parkinson's disease, comprising administering to the subject a therapeutically effective amount of a pharmaceutical composition as described herein.

In some embodiments, the disclosure provides a method of improving learning and working memory in a subject having Mild Cognitive Impairment (MCI) associated with Parkinson's disease, comprising administering to the subject a therapeutically effective amount of a pharmaceutical composition as described herein.

In some embodiments, the disclosure provides a method of improving cognition in a subject having Parkinson's disease, comprising administering to the subject a therapeutically effective amount of a pharmaceutical composition as described herein.

In some embodiments, the disclosure provides a method for slowing cognitive decline in a subject having Parkinson's disease, comprising administering to the subject a therapeutically effective amount of a pharmaceutical composition as described herein.

In one aspect, the disclosure provides a pharmaceutical composition as described herein, for use in treating or preventing Parkinson's disease in a subject. In some embodiments, the disclosure provides a pharmaceutical composition as disclosed herein, for use in treating Parkinson's disease in a subject. In some embodiments, the disclosure provides a pharmaceutical composition as disclosed herein, for use in preventing Parkinson's disease in a subject.

In some embodiments, the disclosure provides a pharmaceutical composition as disclosed herein, for use in treating cognitive impairment in a subject having Parkinson's disease.

In some embodiments, the disclosure provides a pharmaceutical composition as disclosed herein, for use in treating Mild Cognitive Impairment (MCI) associated with Parkinson's disease in a subject.

In some embodiments, the disclosure provides a pharmaceutical composition as disclosed herein, for use in improving executive function in a subject having Mild Cognitive Impairment (MCI) associated with Parkinson's disease.

In some embodiments, the disclosure provides a pharmaceutical composition as disclosed herein, for use in improving working memory in a subject having Mild Cognitive Impairment (MCI) associated with Parkinson's disease.

In some embodiments, the disclosure provides a pharmaceutical composition as disclosed herein, for use in improving learning in a subject having Mild Cognitive Impairment (MCI) associated with Parkinson's disease.

In some embodiments, the disclosure provides a pharmaceutical composition as disclosed herein, for use in improving learning and working memory in a subject having Mild Cognitive Impairment (MCI) associated with Parkinson's disease.

In some embodiments, the disclosure provides a pharmaceutical composition as disclosed herein, for use in improving cognition in a subject having Parkinson's disease.

In some embodiments, the disclosure provides a pharmaceutical composition as disclosed herein, for use in slowing cognitive decline in a subject having Parkinson's disease.

In one aspect, the disclosure provides a use of a pharmaceutical composition as disclosed herein for the manufacture of a medicament for treating or preventing Parkinson's disease in a subject. In some embodiments, the disclosure provides a use of a pharmaceutical composition as disclosed herein for the manufacture of a medicament for treating Parkinson's disease in a subject. In some embodiments, the disclosure provides a use of a pharmaceutical composition as disclosed herein for the manufacture of a medicament for preventing Parkinson's disease in a subject.

In some embodiments, the disclosure provides a use of a pharmaceutical composition as disclosed herein for the manufacture of a medicament for treating cognitive impairment in a subject having Parkinson's disease.

In some embodiments, the disclosure provides a use of a pharmaceutical composition as disclosed herein for the manufacture of a medicament for treating Mild Cognitive Impairment (MCI) associated with Parkinson's disease in a subject.

In some embodiments, the disclosure provides a use of a pharmaceutical composition as disclosed herein for the manufacture of a medicament for improving executive function in a subject having Mild Cognitive Impairment (MCI) associated with Parkinson's disease.

In some embodiments, the disclosure provides a se of a pharmaceutical composition as disclosed herein for the manufacture of a medicament for improving working memory in a subject having Mild Cognitive Impairment (MCI) associated with Parkinson's disease.

In some embodiments, the disclosure provides a use of a pharmaceutical composition as disclosed herein for the manufacture of a medicament for improving learning in a subject having Mild Cognitive Impairment (MCI) associated with Parkinson's disease.

In some embodiments, the disclosure provides a use of a pharmaceutical composition as disclosed herein for the manufacture of a medicament for improving learning and working memory in a subject having Mild Cognitive Impairment (MCI) associated with Parkinson's disease.

In some embodiments, the disclosure provides a use of a pharmaceutical composition as disclosed herein for the manufacture of a medicament for improving cognition in a subject having Parkinson's disease.

In some embodiments, the disclosure provides a use of a pharmaceutical composition as disclosed herein for the manufacture of a medicament for slowing cognitive decline in a subject having Parkinson's disease.

In some embodiments, the methods disclosed herein comprise administering the pharmaceutical composition described herein in combination with other therapeutic agents used for treating Parkinson's disease.

### Huntington's Disease

Huntington's Disease is an inherited disease that causes nerve cells in the brain to waste away. Symptoms include uncontrolled movements, clumsiness, and balance problems. Huntington's disease can hinder walk, talk, and swallowing.

In one aspect, the disclosure provides a method of treating or preventing Huntington's disease in a subject, comprising administering to the subject a therapeutically effective amount of a pharmaceutical composition as described herein.

In some embodiments, the disclosure provides a method of treating Huntington's disease in a subject, comprising administering to the subject a therapeutically effective amount of a pharmaceutical composition as described herein. In some embodiments, the disclosure provides a method of preventing Huntington's disease in a subject, comprising administering to the subject a therapeutically effective amount of a pharmaceutical composition as described herein.

In some embodiments, the disclosure provides a method of treating cognitive impairment in a subject having Huntington's disease, comprising administering to the subject a therapeutically effective amount of a pharmaceutical composition as described herein.

In some embodiments, the disclosure provides a method of treating Mild Cognitive Impairment (MCI) associated with Huntington's disease in a subject, comprising administering to the subject a therapeutically effective amount of a pharmaceutical composition as described herein.

In some embodiments, the disclosure provides a method of improving executive function in a subject having Mild Cognitive Impairment (MCI) associated with Huntington's disease, comprising administering to the subject a therapeutically effective amount of a pharmaceutical composition as described herein.

In some embodiments, the disclosure provides a method of improving working memory in a subject having Mild Cognitive Impairment (MCI) associated with Huntington's disease, comprising administering to the subject a therapeutically effective amount of a pharmaceutical composition as described herein.

In some embodiments, the disclosure provides a method of improving learning in a subject having Mild Cognitive Impairment (MCI) associated with Huntington's disease, comprising administering to the subject a therapeutically effective amount of a pharmaceutical composition as described herein.

In some embodiments, the disclosure provides a method of improving learning and working memory in a subject having Mild Cognitive Impairment (MCI) associated with Huntington's disease, comprising administering to the subject a therapeutically effective amount of a pharmaceutical composition as described herein.

In some embodiments, the disclosure provides a method of improving cognition in a subject having Huntington's disease, comprising administering to the subject a therapeutically effective amount of a pharmaceutical composition as described herein.

In one aspect, the disclosure provides a pharmaceutical composition as described herein, for use in treating or preventing Huntington's disease in a subject. In some embodiments, the disclosure provides a pharmaceutical composition as disclosed herein, for use in treating Huntington's disease in a subject. In some embodiments, the disclosure provides a pharmaceutical composition as disclosed herein, for use in preventing Huntington's disease in a subject.

In some embodiments, the disclosure provides a pharmaceutical composition as disclosed herein, for use in treating cognitive impairment in a subject having Huntington's disease.

In some embodiments, the disclosure provides a pharmaceutical composition as disclosed herein, for use in treating Mild Cognitive Impairment (MCI) associated with Huntington's disease in a subject.

In some embodiments, the disclosure provides a pharmaceutical composition as disclosed herein, for use in improving executive function in a subject having Mild Cognitive Impairment (MCI) associated with Huntington's disease.

In some embodiments, the disclosure provides a pharmaceutical composition as disclosed herein, for use in improving working memory in a subject having Mild Cognitive Impairment (MCI) associated with Huntington's disease.

In some embodiments, the disclosure provides a pharmaceutical composition as disclosed herein, for use in improving learning in a subject having Mild Cognitive Impairment (MCI) associated with Huntington's disease.

In some embodiments, the disclosure provides a pharmaceutical composition as disclosed herein, for use in improving learning and working memory in a subject having Mild Cognitive Impairment (MCI) associated with Huntington's disease.

In some embodiments, the disclosure provides a pharmaceutical composition as disclosed herein, for use in improving cognition in a subject having Huntington's disease.

In some embodiments, the disclosure provides a pharmaceutical composition as disclosed herein, for use in slowing cognitive decline in a subject having Huntington's disease.

In some embodiments, the disclosure provides a method for slowing cognitive decline in a subject having Huntington's disease, comprising administering to the subject a therapeutically effective amount of a pharmaceutical composition as described herein.

In one aspect, the disclosure provides a use of a pharmaceutical composition as disclosed herein for the manufacture of a medicament for treating or preventing Huntington's disease in a subject. In some embodiments, the disclosure provides a use of a pharmaceutical composition as disclosed herein for the manufacture of a medicament for treating Huntington's disease in a subject. In some embodiments, the disclosure provides a use of a pharmaceutical composition as disclosed herein for the manufacture of a medicament for preventing Huntington's disease in a subject.

In some embodiments, the disclosure provides a use of a pharmaceutical composition as disclosed herein for the manufacture of a medicament for treating cognitive impairment in a subject having Huntington's disease.

In some embodiments, the disclosure provides a use of a pharmaceutical composition as disclosed herein for the manufacture of a medicament for treating Mild Cognitive Impairment (MCI) associated with Huntington's disease in a subject.

In some embodiments, the disclosure provides a use of a pharmaceutical composition as disclosed herein for the manufacture of a medicament for improving executive function in a subject having Mild Cognitive Impairment (MCI) associated with Huntington's disease.

In some embodiments, the disclosure provides a se of a pharmaceutical composition as disclosed herein for the manufacture of a medicament for improving working memory in a subject having Mild Cognitive Impairment (MCI) associated with Huntington's disease.

In some embodiments, the disclosure provides a use of a pharmaceutical composition as disclosed herein for the manufacture of a medicament for improving learning in a subject having Mild Cognitive Impairment (MCI) associated with Huntington's disease.

In some embodiments, the disclosure provides a use of a pharmaceutical composition as disclosed herein for the manufacture of a medicament for improving learning and working memory in a subject having Mild Cognitive Impairment (MCI) associated with Huntington's disease.

In some embodiments, the disclosure provides a use of a pharmaceutical composition as disclosed herein for the manufacture of a medicament for improving cognition in a subject having Huntington's disease.

In some embodiments, the disclosure provides a use of a pharmaceutical composition as disclosed herein for the manufacture of a medicament for slowing cognitive decline in a subject having Huntington's disease.

In some embodiments, the methods disclosed herein comprise administering the pharmaceutical composition described herein in combination with other therapeutic agents used for treating Huntington's disease. Examples of such other therapeutic agents include but are not limited to chorea medications (e.g., tetrabenazine (TBZ), deutetetrabenazine (DBZ)), antipsychotics (e.g., olanzapine, risperidone), antidepressants (citalopram, fluoxetine, sertraline) and mood stabilizers (e.g., lamotrigine, carbamazepine).

### Alzheimer's Disease

Alzheimer's disease (AD) is an age-related neurodegenerative disease. Clinically, this disorder is characterized by global cognitive dysfunction, especially memory loss, behavior and personality changes. AD progression has been associated with a gradual damage in function and structure in the hippocampus and neocortex, the vulnerable brain areas used for memory and cognition.

In one aspect, the disclosure provides a method of treating or preventing Alzheimer's disease in a subject, comprising administering to the subject a therapeutically effective amount of a pharmaceutical composition as described herein.

In some embodiments, the disclosure provides a method of treating Alzheimer's disease in a subject, comprising administering to the subject a therapeutically effective amount of a pharmaceutical composition as described herein. In some embodiments, the disclosure provides a method of preventing Alzheimer's disease in a subject, comprising administering to the subject a therapeutically effective amount of a pharmaceutical composition as described herein.

In some embodiments, the disclosure provides a method of treating cognitive impairment in a subject having Alzheimer's disease, comprising administering to the subject a therapeutically effective amount of a pharmaceutical composition as described herein.

In some embodiments, the disclosure provides a method of treating Mild Cognitive Impairment (MCI) associated with Alzheimer's disease in a subject, comprising administering to the subject a therapeutically effective amount of a pharmaceutical composition as described herein.

In some embodiments, the disclosure provides a method of improving executive function in a subject having Mild Cognitive Impairment (MCI) associated with Alzheimer's disease, comprising administering to the subject a therapeutically effective amount of a pharmaceutical composition as described herein.

In some embodiments, the disclosure provides a method of improving working memory in a subject having Mild Cognitive Impairment (MCI) associated with Alzheimer's disease, comprising administering to the subject a therapeutically effective amount of a pharmaceutical composition as described herein.

In some embodiments, the disclosure provides a method of improving learning in a subject having Mild Cognitive Impairment (MCI) associated with Alzheimer's disease, comprising administering to the subject a therapeutically effective amount of a pharmaceutical composition as described herein.

In some embodiments, the disclosure provides a method of improving learning and working memory in a subject having Mild Cognitive Impairment (MCI) associated with Alzheimer's disease, comprising administering to the subject a therapeutically effective amount of a pharmaceutical composition as described herein.

In some embodiments, the disclosure provides a method of improving cognition in a subject having Alzheimer's disease, comprising administering to the subject a therapeutically effective amount of a pharmaceutical composition as described herein.

In some embodiments, the disclosure provides a method for slowing cognitive decline in a subject having Alzheimer's disease, comprising administering to the subject a therapeutically effective amount of a pharmaceutical composition as described herein.

In some embodiments, the disclosure provides a method of treating mild dementia associated with Alzheimer's disease, comprising administering to the subject a therapeutically effective amount of a pharmaceutical composition as described herein.

In some embodiments, the disclosure provides a method of improving executive function in a subject having mild dementia associated with Alzheimer's disease, comprising administering to the subject a therapeutically effective amount of a pharmaceutical composition as described herein.

In some embodiments, the disclosure provides a method of improving working memory in a subject having mild dementia associated with Alzheimer's disease, comprising administering to the subject a therapeutically effective amount of a pharmaceutical composition as described herein.

In some embodiments, the disclosure provides a method of improving learning in a subject having mild dementia associated with Alzheimer's disease, comprising administering to the subject a therapeutically effective amount of a pharmaceutical composition as described herein.

In some embodiments, the disclosure provides a method of improving learning and working memory in a subject having mild dementia associated with Alzheimer's disease, comprising administering to the subject a therapeutically effective amount of a pharmaceutical composition as described herein.

In one aspect, the disclosure provides a pharmaceutical composition as described herein, for use in treating or preventing Alzheimer's disease in a subject. In some embodiments, the disclosure provides a pharmaceutical composition as disclosed herein, for use in treating Alzheimer's disease in a subject. In some embodiments, the disclosure provides a pharmaceutical composition as disclosed herein, for use in preventing Alzheimer's disease in a subject.

In some embodiments, the disclosure provides a pharmaceutical composition as disclosed herein, for use in treating cognitive impairment in a subject having Alzheimer's disease.

In some embodiments, the disclosure provides a pharmaceutical composition as disclosed herein, for use in treating Mild Cognitive Impairment (MCI) associated with Alzheimer's disease in a subject.

In some embodiments, the disclosure provides a pharmaceutical composition as disclosed herein, for use in improving executive function in a subject having Mild Cognitive Impairment (MCI) associated with Alzheimer's disease.

In some embodiments, the disclosure provides a pharmaceutical composition as disclosed herein, for use in improving working memory in a subject having Mild Cognitive Impairment (MCI) associated with Alzheimer's disease.

In some embodiments, the disclosure provides a pharmaceutical composition as disclosed herein, for use in improving learning in a subject having Mild Cognitive Impairment (MCI) associated with Alzheimer's disease.

In some embodiments, the disclosure provides a pharmaceutical composition as disclosed herein, for use in improving learning and working memory in a subject having Mild Cognitive Impairment (MCI) associated with Alzheimer's disease.

In some embodiments, the disclosure provides a pharmaceutical composition as disclosed herein, for use in improving cognition in a subject having Alzheimer's disease.

In some embodiments, the disclosure provides a pharmaceutical composition as disclosed herein, for use in slowing cognitive decline in a subject having Alzheimer's disease.

In some embodiments, the disclosure provides a pharmaceutical composition as disclosed herein, for use in treating mild dementia associated with Alzheimer's Disease.

In some embodiments, the disclosure provides a pharmaceutical composition as disclosed herein, for use in improving executive function in a subject having mild dementia associated with Alzheimer's disease.

In some embodiments, the disclosure provides a pharmaceutical composition as disclosed herein, for use in improving working memory in a subject having mild dementia associated with Alzheimer's disease.

In some embodiments, the disclosure provides a pharmaceutical composition as disclosed herein, for use in improving learning in a subject having mild dementia associated with Alzheimer's disease.

In some embodiments, the disclosure provides a pharmaceutical composition as disclosed herein, for use in improving learning and working memory in a subject having mild dementia associated with Alzheimer's disease.

In one aspect, the disclosure provides a use of a pharmaceutical composition as disclosed herein for the manufacture of a medicament for treating or preventing Alzheimer's disease in a subject. In some embodiments, the disclosure provides a use of a pharmaceutical composition as disclosed herein for the manufacture of a medicament for treating Alzheimer's disease in a subject. In some embodiments, the disclosure provides a use of a pharmaceutical composition as disclosed herein for the manufacture of a medicament for preventing Alzheimer's disease in a subject.

In some embodiments, the disclosure provides a use of a pharmaceutical composition as disclosed herein for the manufacture of a medicament for treating cognitive impairment in a subject having Alzheimer's disease.

In some embodiments, the disclosure provides a use of a pharmaceutical composition as disclosed herein for the manufacture of a medicament for treating Mild Cognitive Impairment (MCI) associated with Alzheimer's disease in a subject.

In some embodiments, the disclosure provides a use of a pharmaceutical composition as disclosed herein for the manufacture of a medicament for improving executive function in a subject having Mild Cognitive Impairment (MCI) associated with Alzheimer's disease.

In some embodiments, the disclosure provides a se of a pharmaceutical composition as disclosed herein for the manufacture of a medicament for improving working memory in a subject having Mild Cognitive Impairment (MCI) associated with Alzheimer's disease.

In some embodiments, the disclosure provides a use of a pharmaceutical composition as disclosed herein for the manufacture of a medicament for improving learning in a subject having Mild Cognitive Impairment (MCI) associated with Alzheimer's disease.

In some embodiments, the disclosure provides a use of a pharmaceutical composition as disclosed herein for the manufacture of a medicament for improving learning and working memory in a subject having Mild Cognitive Impairment (MCI) associated with Alzheimer's disease.

In some embodiments, the disclosure provides a use of a pharmaceutical composition as disclosed herein for the manufacture of a medicament for improving cognition in a subject having Alzheimer's disease.

In some embodiments, the disclosure provides a use of a pharmaceutical composition as disclosed herein for the manufacture of a medicament for slowing cognitive decline in a subject having Alzheimer's disease.

In some embodiments, the disclosure provides a use of a pharmaceutical composition as disclosed herein for the manufacture of a medicament for treating mild dementia associated with Alzheimer's Disease.

In some embodiments, the disclosure provides a use of a pharmaceutical composition as disclosed herein for the manufacture of a medicament for improving executive function in a subject having mild dementia associated with Alzheimer's disease.

In some embodiments, the disclosure provides a use of a pharmaceutical composition as disclosed herein for the manufacture of a medicament for improving working memory in a subject having mild dementia associated with Alzheimer's disease.

In some embodiments, the disclosure provides a use of a pharmaceutical composition as disclosed herein for the manufacture of a medicament for improving learning in a subject having mild dementia associated with Alzheimer's disease.

In some embodiments, the disclosure provides a use of a pharmaceutical composition as disclosed herein for the manufacture of a medicament for improving learning and working memory in a subject having mild dementia associated with Alzheimer's disease.

In some embodiments, the methods disclosed herein comprise administering the pharmaceutical composition described herein in combination with other therapeutic agents used for treating Alzheimer's disease.

In some embodiments, the additional therapeutic agent for treating Alzheimer's disease is an anti-amyloid beta antibody or antigen-binding fragment thereof useful in the compositions, kits and methods of the present disclosure is an antibody. Any anti-amyloid beta antibody or antigen binding fragment thereof, including those disclosed in U.S. Pat. Nos. 10,842,871, 10,131,708, 9,828,420, and 8,906,367, and US patent publication 2015/013155267, the disclosures of all of which are incorporated by reference herein, may be used in the pharmaceutical compositions, kits and methods disclosed herein.

More specifically, the additional therapeutic agent for treating Alzheimer's disease comprises an anti-amyloid beta antibody, antigen-binding fragment (or portion) thereof, the light chain of the antibody, the heavy chain of the antibody, and fragments of these light chains or heavy chains. The anti-amyloid beta antibodies and antigen-binding fragments thereof disclosed herein also include antibodies lacking the heavy and/or light chain signal sequences and glycosylated antibodies. The anti-amyloid beta antibodies and antigen-binding fragments thereof disclosed herein also include precursor antibodies, nonglycosylated antibodies, and antibodies whose heavy and/or light chains comprise signal sequences.

The antibodies and antigen-binding fragments thereof described herein can be purified and/or isolated using known techniques. Antibodies or portions that are “purified” or “isolated” have been at least partially separated away from molecules (e.g., peptides) of their source of origin (e.g., the supernatant of cells; in a mixture such as in a mixture of antibodies in a library; etc.), and include antibodies obtained by any other suitable methods. Isolated antibodies include substantially pure (e.g., essentially pure) antibodies, as well as antibodies produced by chemical synthesis, recombinant techniques and a combination thereof.

An anti-amyloid beta antibody or antigen-binding fragment thereof, may be human. In some embodiments, an anti-amyloid beta antibody or antigen-binding fragment thereof, of the disclosure is chimeric. In some embodiments, the chimeric anti-amyloid beta antibody or antigen-binding fragment thereof, comprises a chimeric IgH chain and a human Igκ chain. In some embodiments, the chimeric anti-amyloid beta antibody or antigen-binding fragment thereof, comprises a chimeric IgH chain and a human Igλ chain. In some embodiments, the chimeric anti-S1 antibody comprises human and mouse sequences.

In some embodiments, the anti-amyloid beta antibody or antigen-binding fragment thereof comprises one to six complementarity determining regions (CDRs) disclosed herein.

In some embodiments, the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof. In some embodiments, the anti-amyloid beta antibody or antigen-binding fragment thereof is bapineuzumab or an antigen-binding fragments thereof. In some embodiments, the anti-amyloid beta antibody or antigen-binding fragment thereof is solanezumab, or an antigen-binding fragments thereof. In some embodiments, the anti-amyloid beta antibody or antigen-binding fragment thereof is gantenerumab or an antigen-binding fragments thereof. In some embodiments, the anti-amyloid beta antibody or antigen-binding fragment thereof is crenezumab or an antigen-binding fragments thereof. In some embodiments, the anti-amyloid beta antibody or antigen-binding fragment thereof is ponezumab or an antigen-binding fragments thereof. In some embodiments, the anti-amyloid beta antibody or antigen-binding fragment thereof is lecanemab (BAN2401) or an antigen-binding fragments thereof. In some embodiments, the anti-amyloid beta antibody or antigen-binding fragment thereof is aducanumab or an antigen-binding fragments thereof.

In some embodiments, the anti-amyloid beta antibody or antigen binding fragment thereof binds to the same conformational epitope of amyloid beta to which any one of bapineuzumab, solanezumab, gantenerumab, crenezumab, ponezumab, lecanemab (BAN2401), or aducanumab binds. In some embodiments, the anti-amyloid beta antibody or antigen binding fragment thereof binds to the same conformational epitope of amyloid beta to which bapineuzumab binds. In some embodiments, the anti-amyloid beta antibody or antigen binding fragment thereof binds to the same conformational epitope of amyloid beta to which solanezumab binds. In some embodiments, the anti-amyloid beta antibody or antigen binding fragment thereof binds to the same conformational epitope of amyloid beta to which gantenerumab binds. In some embodiments, the anti-amyloid beta antibody or antigen binding fragment thereof binds to the same conformational epitope of amyloid beta to which crenezumab binds. In some embodiments, the anti-amyloid beta antibody or antigen binding fragment thereof binds to the same conformational epitope of amyloid beta to which ponezumab binds. In some embodiments, the anti-amyloid beta antibody or antigen binding fragment thereof binds to the same conformational epitope of amyloid beta to which lecanemab (BAN2401) binds. In some embodiments, the anti-amyloid beta antibody or antigen binding fragment thereof binds to the same conformational epitope of amyloid beta to which aducanumab binds.

In some embodiments, the anti-amyloid beta antibody or antigen binding fragment thereof competitively inhibits binding of any one of bapineuzumab, solanezumab, gantenerumab, crenezumab, ponezumab, lecanemab (BAN2401), or aducanumab to amyloid beta. In some embodiments, the anti-amyloid beta antibody or antigen binding fragment thereof competitively inhibits binding of bapineuzumab to amyloid beta. In some embodiments, the anti-amyloid beta antibody or antigen binding fragment thereof competitively inhibits binding of solanezumab to amyloid beta. In some embodiments, the anti-amyloid beta antibody or antigen binding fragment thereof competitively inhibits binding of gantenerumab to amyloid beta. In some embodiments, the anti-amyloid beta antibody or antigen binding fragment thereof competitively inhibits binding of ponezumab to amyloid beta. In some embodiments, the anti-amyloid beta antibody or antigen binding fragment thereof competitively inhibits binding of lecanemab (BAN2401) to amyloid beta. In some embodiments, the anti-amyloid beta antibody or antigen binding fragment thereof competitively inhibits binding of aducanumab to amyloid beta.

In some embodiments, the anti-amyloid beta antibody or antigen-binding fragment thereof comprises the 6 CDRs of any one of the anti-amyloid beta antibodies disclosed herein as determined by Kabat, Chothia or IMTG nomenclature. In some embodiments, the anti-amyloid beta antibody or antigen-binding fragment thereof comprises the 6 CDRs of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the 6 CDRs of bapineuzumab. In some embodiments, the anti-amyloid beta antibody or antigen-binding fragment thereof comprises the 6 CDRs of solanezumab. In some embodiments, the anti-amyloid beta antibody or antigen-binding fragment thereof comprises the 6 CDRs of gantenerumab. In some embodiments, the anti-amyloid beta antibody or antigen-binding fragment thereof comprises the 6 CDRs of crenezumab. In some embodiments, the anti-amyloid beta antibody or antigen-binding fragment thereof comprises the 6 CDRs of ponezumab. In some embodiments, the anti-amyloid beta antibody or antigen-binding fragment thereof comprises the 6 CDRs of lecanemab (BAN2401). In some embodiments, the anti-amyloid beta antibody or antigen-binding fragment thereof comprises the 6 CDRs of aducanumab.

### Dosing Regimens

In some embodiments, Compound I or a pharmaceutically acceptable salt thereof is administered to the subject at a total daily dose of from about 0.3 mg to about 2.1 mg. In some embodiments, Compound I or a pharmaceutically acceptable salt thereof is administered to the subject at a total daily dose of from about 0.3 mg to about 1.8 mg. In some embodiments, Compound I or a pharmaceutically acceptable salt thereof is administered to the subject at a total daily dose of from about 0.3 mg to about 1.5 mg. In some embodiments, Compound I or a pharmaceutically acceptable salt thereof is administered to the subject at a total daily dose of from about 0.3 mg to about 1.2 mg. In some embodiments, Compound I or a pharmaceutically acceptable salt thereof is administered to the subject at a total daily dose of from about 0.6 mg to about 1.2 mg. In some embodiments, Compound I or a pharmaceutically acceptable salt thereof is administered to the subject at a total daily dose of from about 0.6 mg to about 1.8 mg. In some embodiments, Compound I or a pharmaceutically acceptable salt thereof is administered to the subject at a total daily dose of from about 0.9 mg to about 1.2 mg. In some embodiments, Compound I or a pharmaceutically acceptable salt thereof is administered to the subject at a total daily dose from about 0.9 mg to 1.5 mg. In some embodiments, Compound I or a pharmaceutically acceptable salt thereof is administered to the subject at a total daily dose of about 0.3 mg, about 0.6 mg, about 0.9 mg, about 1.2 mg, about 1.5 mg, about 1.8 mg, about 2.1 mg. In some embodiments, Compound I or a pharmaceutically acceptable salt thereof is administered to the subject a total daily dose of about 0.3 mg. In some embodiments, Compound I or a pharmaceutically acceptable salt thereof is administered to the subject a total daily dose of about 0.6 mg. In some embodiments, Compound I or a pharmaceutically acceptable salt thereof is administered to the subject a total daily dose of about 0.9 mg. In some embodiments, Compound I or a pharmaceutically acceptable salt thereof is administered to the subject a total daily dose of about 1.2 mg.

In some embodiments, Compound I or a pharmaceutically acceptable salt thereof is administered to the subject at a total daily dose of from 0.3 mg to 2.1 mg. In some embodiments, Compound I or a pharmaceutically acceptable salt thereof is administered to the subject at a total daily dose of from 0.3 mg to 1.8 mg. In some embodiments, Compound I or a pharmaceutically acceptable salt thereof is administered to the subject at a total daily dose of from 0.3 mg to 1.5 mg. In some embodiments, Compound I or a pharmaceutically acceptable salt thereof is administered to the subject at a total daily dose of from 0.3 mg to 1.2 mg. In some embodiments, Compound I or a pharmaceutically acceptable salt thereof is administered to the subject at a total daily dose of from 0.6 mg to 1.2 mg. In some embodiments, Compound I or a pharmaceutically acceptable salt thereof is administered to the subject at a total daily dose of from 0.6 mg to 1.8 mg. In some embodiments, Compound I or a pharmaceutically acceptable salt thereof is administered to the subject at a total daily dose of from 0.9 mg to 1.2 mg. In some embodiments, Compound I or a pharmaceutically acceptable salt thereof is administered to the subject at a total daily dose from 0.9 mg to 1.5 mg. In some embodiments, Compound I or a pharmaceutically acceptable salt thereof is administered to the subject at a total daily dose of 0.3 mg, 0.6 mg, 0.9 mg, 1.2 mg, 1.5 mg, 1.8 mg, 2.1 mg. In some embodiments, Compound I or a pharmaceutically acceptable salt thereof is administered to the subject a total daily dose of 0.3 mg. In some embodiments, Compound I or a pharmaceutically acceptable salt thereof is administered to the subject a total daily dose of 0.6 mg. In some embodiments, Compound I or a pharmaceutically acceptable salt thereof is administered to the subject a total daily dose of 0.9 mg. In some embodiments, Compound I or a pharmaceutically acceptable salt thereof is administered to the subject a total daily dose of 1.2 mg.

In some embodiments, Compound I is administered to the subject at a total daily dose of from about 0.3 mg to about 2.1 mg. In some embodiments, Compound I is administered to the subject at a total daily dose of from about 0.3 mg to about 1.8 mg. In some embodiments, Compound I is administered to the subject at a total daily dose of from about 0.3 mg to about 1.5 mg. In some embodiments, Compound I is administered to the subject at a total daily dose of from about 0.3 mg to about 1.2 mg. In some embodiments, Compound I is administered to the subject at a total daily dose of from about 0.6 mg to about 1.2 mg. In some embodiments, Compound I is administered to the subject at a total daily dose of from about 0.6 mg to about 1.8 mg. In some embodiments, Compound I is administered to the subject at a total daily dose of from about 0.9 mg to about 1.2 mg. In some embodiments, Compound I is administered to the subject at a total daily dose from about 0.9 mg to 1.5 mg. In some embodiments, Compound I is administered to the subject at a total daily dose of about 0.3 mg, about 0.6 mg, about 0.9 mg, about 1.2 mg, about 1.5 mg, about 1.8 mg, about 2.1 mg. In some embodiments, Compound I is administered to the subject a total daily dose of about 0.3 mg. In some embodiments, Compound I is administered to the subject a total daily dose of about 0.6 mg. In some embodiments, Compound I is administered to the subject a total daily dose of about 0.9 mg. In some embodiments, Compound I is administered to the subject a total daily dose of about 1.2 mg.

In some embodiments, Compound I is administered to the subject at a total daily dose of from 0.3 mg to 2.1 mg. In some embodiments, Compound I is administered to the subject at a total daily dose of from 0.3 mg to 1.8 mg. In some embodiments, Compound I is administered to the subject at a total daily dose of from 0.3 mg to 1.5 mg. In some embodiments, Compound I is administered to the subject at a total daily dose of from 0.3 mg to 1.2 mg. In some embodiments, Compound I is administered to the subject at a total daily dose of from 0.6 mg to 1.2 mg. In some embodiments, Compound I is administered to the subject at a total daily dose of from 0.6 mg to 1.8 mg. In some embodiments, Compound I is administered to the subject at a total daily dose of from 0.9 mg to 1.2 mg. In some embodiments, Compound I is administered to the subject at a total daily dose from 0.9 mg to 1.5 mg. In some embodiments, Compound I is administered to the subject at a total daily dose of 0.3 mg, 0.6 mg, 0.9 mg, 1.2 mg, 1.5 mg, 1.8 mg, 2.1 mg. In some embodiments, Compound I is administered to the subject a total daily dose of 0.3 mg. In some embodiments, Compound I is administered to the subject a total daily dose of 0.6 mg. In some embodiments, Compound I is administered to the subject a total daily dose of 0.9 mg. In some embodiments, Compound I is administered to the subject a total daily dose of 1.2 mg.

In some embodiments, Compound I or a pharmaceutically acceptable salt thereof is administered as a loading dose of about 1.2 mg daily starting on day 1 to about day 26 to 30 of treatment, and thereafter Compound I or a pharmaceutically acceptable salt thereof is administered as a maintenance dose of about 0.9 mg daily up to day 84 of the treatment. In some embodiments, Compound I or a pharmaceutically acceptable salt thereof is administered as a loading dose of 1.2 mg daily starting on day 1 to about day 26 to 30 of treatment, and thereafter Compound I or a pharmaceutically acceptable salt thereof is administered as a maintenance dose of 0.9 mg daily for up to day 84 of the treatment. In some embodiments, Compound I or a pharmaceutically acceptable salt thereof is administered as a loading dose of about 1.2 mg daily starting on day 1 to about day 26 to 30 of treatment, and thereafter Compound I or a pharmaceutically acceptable salt thereof is administered as a maintenance dose of about 0.9 mg daily for the remainder of the treatment. In some embodiments, Compound I or a pharmaceutically acceptable salt thereof is administered as a loading dose of 1.2 mg daily starting on day 1 to about day 26 to 30 of treatment, and thereafter Compound I or a pharmaceutically acceptable salt thereof is administered as a maintenance dose of 0.9 mg daily for the remainder of the treatment.

In some embodiments, Compound I or a pharmaceutically acceptable salt thereof or a pharmaceutically acceptable salt thereof is administered as a loading dose of 1.2 mg daily starting on day 1 to day 26 to 30 of treatment, and thereafter Compound I or a pharmaceutically acceptable salt thereof is administered as a maintenance dose of 0.9 mg daily up to day 84 of the treatment. In some embodiments, Compound I or a pharmaceutically acceptable salt thereof is administered as a loading dose of 1.2 mg daily starting on day 1 to day 26 to 30 of treatment, and thereafter Compound I or a pharmaceutically acceptable salt thereof is administered as a maintenance dose of 0.9 mg daily for up to day 84 of the treatment. In some embodiments, Compound I or a pharmaceutically acceptable salt thereof is administered as a loading dose of 1.2 mg daily starting on day 1 to day 26 to 30 of treatment, and thereafter Compound I or a pharmaceutically acceptable salt thereof is administered as a maintenance dose of 0.9 mg daily for the remainder of the treatment. In some embodiments, Compound I or a pharmaceutically acceptable salt thereof is administered as a loading dose of 1.2 mg daily starting on day 1 to day 26 to 30 of treatment, and thereafter Compound I or a pharmaceutically acceptable salt thereof is administered as a maintenance dose of 0.9 mg daily for the remainder of the treatment.

In some embodiments, Compound I is administered as a loading dose of about 1.2 mg daily starting on day 1 to about day 26 to 30 of treatment, and thereafter Compound I is administered as a maintenance dose of about 0.9 mg daily up to day 84 of the treatment. In some embodiments, Compound I is administered as a loading dose of 1.2 mg daily starting on day 1 to about day 26 to 30 of treatment, and thereafter Compound I is administered as a maintenance dose of 0.9 mg daily for up to day 84 of the treatment. In some embodiments, Compound I is administered as a loading dose of about 1.2 mg daily starting on day 1 to about day 26 to 30 of treatment, and thereafter Compound I is administered as a maintenance dose of about 0.9 mg daily for the remainder of the treatment. In some embodiments, Compound I is administered as a loading dose of 1.2 mg daily starting on day 1 to about day 26 to 30 of treatment, and thereafter Compound I is administered as a maintenance dose of 0.9 mg daily for the remainder of the treatment.

In some embodiments, Compound I is administered as a loading dose of 1.2 mg daily starting on day 1 to day 26 to 30 of treatment, and thereafter Compound I is administered as a maintenance dose of 0.9 mg daily up to day 84 of the treatment. In some embodiments, Compound I is administered as a loading dose of 1.2 mg daily starting on day 1 to day 26 to 30 of treatment, and thereafter Compound I is administered as a maintenance dose of 0.9 mg daily for up to day 84 of the treatment. In some embodiments, Compound I is administered as a loading dose of 1.2 mg daily starting on day 1 to day 26 to 30 of treatment, and thereafter Compound I is administered as a maintenance dose of 0.9 mg daily for the remainder of the treatment. In some embodiments, Compound I is administered as a loading dose of 1.2 mg daily starting on day 1 to day 26 to 30 of treatment, and thereafter Compound I is administered as a maintenance dose of 0.9 mg daily for the remainder of the treatment.

In some embodiments, Compound I or a pharmaceutically acceptable salt thereof is administered as a loading dose of about 1.2 mg daily starting on day 1 to about day 40 to 44 of treatment, and thereafter Compound I or a pharmaceutically acceptable salt thereof is administered as a maintenance dose of about 0.9 mg daily up to day 84 of the treatment. In some embodiments, Compound I or a pharmaceutically acceptable salt thereof is administered as a loading dose of 1.2 mg daily starting on day 1 to about day 40 to 44 of treatment, and thereafter Compound I or a pharmaceutically acceptable salt thereof is administered as a maintenance dose of 0.9 mg daily for up to day 84 of the treatment. In some embodiments, Compound I or a pharmaceutically acceptable salt thereof is administered as a loading dose of about 1.2 mg daily starting on day 1 to about day 40 to 44 of treatment, and thereafter Compound I or a pharmaceutically acceptable salt thereof is administered as a maintenance dose of about 0.9 mg daily for the remainder of the treatment. In some embodiments, Compound I or a pharmaceutically acceptable salt thereof is administered as a loading dose of 1.2 mg daily starting on day 1 to about day 40 to 44 of treatment, and thereafter Compound I or a pharmaceutically acceptable salt thereof is administered as a maintenance dose of 0.9 mg daily for the remainder of the treatment.

In some embodiments, Compound I or a pharmaceutically acceptable salt thereof is administered as a loading dose of 1.2 mg daily starting on day 1 to day 40 to 44 of treatment, and thereafter Compound I or a pharmaceutically acceptable salt thereof is administered as a maintenance dose of 0.9 mg daily up to day 84 of the treatment. In some embodiments, Compound I or a pharmaceutically acceptable salt thereof is administered as a loading dose of 1.2 mg daily starting on day 1 to day 40 to 44 of treatment, and thereafter Compound I or a pharmaceutically acceptable salt thereof is administered as a maintenance dose of 0.9 mg daily for up to day 84 of the treatment. In some embodiments, Compound I or a pharmaceutically acceptable salt thereof is administered as a loading dose of 1.2 mg daily starting on day 1 to day 40 to 44 of treatment, and thereafter Compound I or a pharmaceutically acceptable salt thereof is administered as a maintenance dose of 0.9 mg daily for the remainder of the treatment. In some embodiments, Compound I or a pharmaceutically acceptable salt thereof is administered as a loading dose of 1.2 mg daily starting on day 1 to day 40 to 44 of treatment, and thereafter Compound I or a pharmaceutically acceptable salt thereof is administered as a maintenance dose of 0.9 mg daily for the remainder of the treatment.

In some embodiments, Compound I is administered as a loading dose of about 1.2 mg daily starting on day 1 to about day 40 to 44 of treatment, and thereafter Compound I is administered as a maintenance dose of about 0.9 mg daily up to day 84 of the treatment. In some embodiments, Compound I is administered as a loading dose of 1.2 mg daily starting on day 1 to about day 40 to 44 of treatment, and thereafter Compound I is administered as a maintenance dose of 0.9 mg daily for up to day 84 of the treatment. In some embodiments, Compound I is administered as a loading dose of about 1.2 mg daily starting on day 1 to about day 40 to 44 of treatment, and thereafter Compound I is administered as a maintenance dose of about 0.9 mg daily for the remainder of the treatment. In some embodiments, Compound I is administered as a loading dose of 1.2 mg daily starting on day 1 to about day 40 to 44 of treatment, and thereafter Compound I is administered as a maintenance dose of 0.9 mg daily for the remainder of the treatment.

In some embodiments, Compound I is administered as a loading dose of 1.2 mg daily starting on day 1 to day 40 to 44 of treatment, and thereafter Compound I is administered as a maintenance dose of 0.9 mg daily up to day 84 of the treatment. In some embodiments, Compound I is administered as a loading dose of 1.2 mg daily starting on day 1 to day 40 to 44 of treatment, and thereafter Compound I is administered as a maintenance dose of 0.9 mg daily for up to day 84 of the treatment. In some embodiments, Compound I is administered as a loading dose of 1.2 mg daily starting on day 1 to day 40 to 44 of treatment, and thereafter Compound I is administered as a maintenance dose of 0.9 mg daily for the remainder of the treatment. In some embodiments, Compound I is administered as a loading dose of 1.2 mg daily starting on day 1 to day 40 to 44 of treatment, and thereafter Compound I is administered as a maintenance dose of 0.9 mg daily for the remainder of the treatment.

In some embodiments, the methods disclosed herein comprise administering to the subject at least one loading dose; and at least one maintenance dose. In some embodiments, the subject is administered a loading dose daily for at least 1, 2, 3, 4, 5, 6, 7, 8, 9 or 10 weeks. In some embodiments, the subject is administered a loading dose daily for at least 2, 4, 6, 8 or 10 weeks. In some embodiments, the subject is administered a loading dose daily for at least 2 weeks. In some embodiments, the subject is administered a loading dose daily for at least 4 weeks. In some embodiments, the subject is administered a loading dose daily for at least 6 weeks. In some embodiments, the subject is administered a loading dose daily for at least 8 weeks. In some embodiments, the subject is administered a loading dose daily for at least 10 weeks.

In some embodiments, the subject is administered a loading dose daily for 2, 4, 6, 8 or 10 weeks. In some embodiments, the subject is administered a loading dose daily for 2 weeks. In some embodiments, the subject is administered a loading dose daily for 4 weeks. In some embodiments, the subject is administered a loading dose daily for 6 weeks. In some embodiments, the subject is administered a loading dose daily for 8 weeks. In some embodiments, the subject is administered a loading dose daily for 10 weeks.

In some embodiments, the methods disclosed herein comprise administering to the subject at least one loading dose; and at least one maintenance dose. In some embodiments, the subject is administered a loading dose weekly for at least 1, 2, 3, 4, 5, 6, 7, 8, 9 or 10 weeks. In some embodiments, the subject is administered a loading dose weekly for at least 2, 4, 6, 8 or 10 weeks. In some embodiments, the subject is administered a loading dose weekly for at least 2 weeks. In some embodiments, the subject is administered a loading dose weekly for at least 4 weeks. In some embodiments, the subject is administered a loading dose weekly for at least 6 weeks. In some embodiments, the subject is administered a loading dose weekly for at least 8 weeks. In some embodiments, the subject is administered a loading dose weekly for at least 10 weeks.

In some embodiments, the subject is administered a loading dose weekly for 2, 4, 6, 8 or 10 weeks. In some embodiments, the subject is administered a loading dose weekly for 2 weeks. In some embodiments, the subject is administered a loading dose weekly for 4 weeks. In some embodiments, the subject is administered a loading dose weekly for 6 weeks. In some embodiments, the subject is administered a loading dose weekly for 8 weeks. In some embodiments, the subject is administered a loading dose weekly for 10 weeks.

In some embodiments, the loading dose is about 1.2 mg daily of Compound I or a pharmaceutically acceptable salt thereof. In some embodiments, the loading dose is 1.2 mg daily of Compound I or a pharmaceutically acceptable salt thereof.

In some embodiments, the loading dose is about 1.2 mg daily of Compound I. In some embodiments, the loading dose is 1.2 mg daily of Compound I.

In some embodiments, the maintenance dose is administered daily. In some embodiments, the maintenance dose is about 0.9 mg daily of Compound I or a pharmaceutically acceptable salt thereof. In some embodiments, the maintenance dose is 0.9 mg daily of Compound I or a pharmaceutically acceptable salt thereof. In some embodiments, the maintenance dose is about 0.6 mg daily of Compound I or a pharmaceutically acceptable salt thereof. In some embodiments, the maintenance dose is 0.6 mg daily of Compound I or a pharmaceutically acceptable salt thereof.

In some embodiments, the maintenance dose is administered daily. In some embodiments, the maintenance dose is about 0.9 mg daily of Compound I. In some embodiments, the maintenance dose is 0.9 mg daily of Compound I. In some embodiments, the maintenance dose is about 0.6 mg daily of Compound I. In some embodiments, the maintenance dose is 0.6 mg daily of Compound I.

### EXAMPLES

In order that the invention described herein may be more fully understood, the following examples are set forth. The Examples are offered for illustrative purposes and are not to be construed in any way as limiting the scope of the invention.

### Example 1: Preparation of Softgels

Standard methodologies for preparing softgels were utilized to prepare two formulations (Formulation 1 and Formulation 2).

The composition of Formulation 1 is summarized in Table 1:

TABLE 1
Formulation 1 Composition

| Component | % w/w |
|---|---|
| Compound I | 0.30 |
| Glyceryl Monocaprylate | 59.81 |
| Glyceryl Monolinoleate | 19.94 |
| Vitamin E TPGS | 19.94 |
| Butylated Hydroxyanisole (BHA) | 0.01 |

The composition of Formulation 2 is summarized in Table 2:

TABLE 2
Formulation 2 Composition

| Component | % w/w |
|---|---|
| Compound I | 0.30 |
| Glyceryl Monocaprylate | 49.85 |
| Glyceryl Monolinoleate | 22.43 |
| Vitamin E TPGS | 19.94 |
| Stearoyl polyoxy1-32 glycerides | 7.48 |
| Butylated Hydroxyanisole (BHA) | 0.01 |

### Example 2: Evaluation by Non-Sink Dissolution

Formulation 1 was evaluated by dissolution with the conditions described in Table 3. The formulation was run in one dissolution vessel, but two samples were taken at each time point. One sample was filtered, and one sample was not. A sample was also taken at two hours for polarized light microscopy (PLM) evaluation. The dissolution results and observations are detailed in Table 4 Formulation 1 had near full release at 1 hour for both filtered and unfiltered samples. Formulation 1 was at 7800 at 2 hours filtered. The unfiltered results for Formulation 1 were close to full release at greater than 80%. The results show that the filtration step is removing un-solubilized precipitated active as intended.

TABLE 3
Non-Sink Dissolution Conditions

| Parameter | Details |
|---|---|
| Dose | 10 mg (1 gram fill weight soft gel) |
| Dissolution Media | 0.1N HCl (pH 1.2) |
| Volume | 200 mL |
| Temperature | 37° C. |
| Mixing | 50 rpm (Type II paddle) |
| Sampling | 10 mL (at 1 and 2 hrs). |
| Filtration | With and without 1 μm glass fiber filters with first 3 mL |
| | discarded |
| Sample Prep | 2.5 mL sample diluted to 10 mL with 90/10 MeOH/DMSO |

TABLE 4
Non-Sink Dissolution Results and Observations

| | Dissolution Results | | Dissolution Results | | |
|---|---|---|---|---|---|
| | - Filtered - | | - Unfiltered - | | |
| | % of Target | | % of Target | | |
| Formula | 1 hour | 2 hours | 1 hour | 2 hours | Observations |
| Formulation | 96% | 78% | 102% | 88% | Cloudy and well dispersed |
| 1 | | | | | for 2 hours of study. |

### Example 3: Dissolution Evaluation

Dissolution was performed on Formulation 1 and Formulation 2, using the conditions listed in Table 5. Both formulations had greater than 80% Compound I released at the 2-hour time point in 0.1 N HCl (Table 6). Unfiltered results in FeSSIF showed full release through 2-hours, but some precipitate was noted in both samples. The FeSSIF dissolution was repeated with filtration included with results of 92% release for both at 1 hour and 4400 and 5400 at 2 hours for Formulation 1 and Formulation 2, respectively (Table 6).

TABLE 5
Dissolution Conditions of Formulation 1 and Formulation 2 in 0.1N HCl

| Parameter | Details |
|---|---|
| Dose | 10 mg (1 gram fill weight soft gel) |
| Dissolution Media | 0.1N HCl (pH 1.2) or FeSSIFv1 (pH 5.1) |
| Volume | 200 mL |
| Temperature | 37° C. |
| Mixing | 50 rpm (Type II paddle) |
| Sampling | 10 mL (at 1 and 2 hrs) |
| Filtration | With or without 1 μm glass fiber filters with first 3 mL |
| | discarded |
| Sample Prep | 2.5 mL sample diluted to 10 mL with 90/10 MeOH/DMSO |
| Analysis | HPLC-UV (Analysis at 205 nm) |

### and FeSSIFvl

TABLE 6
Dissolution Results of Formulation 1 and
Formulation 2 in 0.1N HCl and FeSSIFv1

| | 0.1N HCl - | | FeSSIFv1 - | | FeSSIFv1 - | |
|---|---|---|---|---|---|---|
| | Unfiltered - | | Unfiltered - | | Filtered - | |
| | % of Target | | % of Target | | % of Target | |
| Formula | 1 hour | 2 hours | 1 hour | 2 hours | 1 hour | 2 hours |
| Formulation | 94% | 84% | 108% | 106% | 92% | 44% |
| 1 | | | | | | |
| Formulation | 84% | 104% | 96% | 98% | 92% | 54% |
| 2 | | | | | | |

### Example 4: Solubility of Polymorphs in Selected Formulations

The equilibrium solubility of Form A and Form C were evaluated in Formulation 1 and Formulation 2. Ideally, the equilibrium solubilities of the anhydrate and hydrate forms should be as close as possible to prevent unwanted active precipitation inside the lipid dosage form during long-term storage. Excess Form C and Form A were added to Formulation 1 and Formulation 2 placebo formulations and were allowed to mix at 25° C. for 48 hours. The saturated samples were then transferred to centrifuge tubes with 0.45 μm PVDF filters and centrifuged at 15000 rpm for 15 minutes at 25° C. The filtrate was analyzed by HIPLC for assay to determine the equilibrium solubility for each form in each formulation. Table 7 details the solubility results for the formulations. Table 7 shows that the equilibrium solubility for both forms is essentially the same for Formulation 1. The anhydrate has an approximately 3000 higher equilibrium solubility than the hydrate form in Formulation 2. This lower solubility level for the hydrate suggests that precipitation of the hydrate could occur for Formulation 2 and should be carefully evaluated during development of this formulation.

TABLE 7
Saturated Solubility of Form A and Form C in Formulations

| | Formulation | Formulation |
|---|---|---|
| Formulation | 1 | 2 |
| Anhydrate (Form A), mg/g | 22.9 | 20.6 |
| Hydrate (Form C), mg/g | 21.2 | 14.6 |

Since the Form A and Form C were of similar overall solubility in the formulations, the dissolution time for both was visually assessed in formulations Formulation 1 and Formulation 2 to see if either form may offer improved processability. Formulation 1 and Formulation 2 placebo formulations were prepared, and 2 mg of each form was added to 1 gram of each formulation followed by a 30 second vortex to distribute the compound. Each preparation was then stirred at 400 rpm at 25° C. and the solubilization of the two forms was tracked visually. The hydrate (Form C) was fully solubilized in both formulations in less than 2 hours. The anhydrate (Form A) took between 3 and 4 hours to fully solubilize in both formulations.

### Example 5: Temperature Cycling and Water Challenge for Selected Formulations

Formulation 1 was exposed to three refrigeration (4° C.)/heat (40° C.) cycles for 24 hours at each condition and to a 5% water challenge at room temperature for 6 days. These studies were performed to assess the robustness of the formulation to temperature cycles that can be experienced during shipping, handling and storage and to water ingress that can occur during the hard shell or softgel encapsulation process. The results for these studies are detailed in Table 8. Both formulations performed well in both evaluations with only a slight separation noted during the third refrigeration cycle. Formulation 1 formulation became uniform and clear once it was warmed up with no precipitation or other changes noted.

TABLE 8
Refrigeration Heat Cycling and Water Challenge Results for Formulation 1

| | Refrigeration Heat Observations (3 | Water Challenge (5% Water |
|---|---|---|
| Formulation | cycles of 4° C. to 40° C. for 24 hrs each) | Spiked and Held for 6 Days) |
| Formulation 1 | Separation with white bottom layer | Remained clear, light yellow |
| Formulation 1 | noted on last freeze. Thawed uniform | solutions without phase |
| (PBO) | and clear. No ppt noted. Placebo | separation throughout |
| | matched active. | evaluation. No ppt. |

### Example 6: Emulsion Droplet Size of Selected Formulations in 0.1 N HCl

Emulsion droplet sizes that resulted from formulation placebo or active (10 mg/g Compound I) dispersed at 1/100 dilution in 0.1 N HCl at 37° C. were assessed by dynamic light scattering (DLS) analysis. Formulations of Formulation 1 and Formulation 2 and their respective placebos were prepared as well as 0.1 N HCl buffer. Formulations and the buffers were incubated to bring them to 37° C. Formulations were added to the buffer at dilutions of 1/100 and briefly shaken until uniform emulsions were formed. The emulsions were allowed to sit at 37° C. to allow air to rise out of the emulsion prior to being analyzed by DLS (Malvern Zetasizer Nano). Table 9 below details experimental details and the results of the analysis. The results show that the average particle size of the emulsion is between 250-350 nanometers. This size is sufficiently small to ensure good dispersion in the stomach to promote drug absorption.

TABLE 9
Formulation 1 and Formulation 2 DLS Parameters and Results

| | Formulations | | | |
|---|---|---|---|---|
| | Formulation | Formulation 1 | Formulation | Formulation 2 |
| Parameters | 1 | (PBO) | 2 | (PBO) |
| Z-Average (d.nm) | 265.0 | 331.2 | 310.0 | 254.2 |
| PDI | 0.402 | 0.469 | 0.316 | 0.363 |
| Temperature (° C.) | 37.0 | 37.0 | 37.0 | 37.0 |
| Duration (seconds) | 60 | 60 | 80 | 90 |
| Count Rate (kcps) | 397.0 | 363.2 | 125.2 | 116.0 |
| Refractive Index | 1.33 | 1.33 | 1.33 | 1.33 |
| Viscosity (cP) | 0.6864 | 0.6864 | 0.6864 | 0.6864 |

### Example 7: Informal Stability

Formulations Formulation 1 and Formulation 2 were evaluated for stability for 4 weeks at 40° C. with and without 5% water. Formulations were prepared at a concentration of 10 mg/g of Compound I. Each formulation was divided in half with one portion spiked with 5% water. Prepared formulations, with and without spiked water, were packaged in sealed amber glass bottles with nitrogen blanketing and were stored at 40° C. for 4 weeks for the study. Samples were analyzed for Compound I content at 0, 2 and 4 weeks. Results are detailed in Table 10. Assay values remained steady for all samples through the duration of the study.

TABLE 10
Informal Stability at 40° C. for Selected Formulations

| | T0 | 2 Weeks | 4 Weeks |
|---|---|---|---|
| | mg/g | mg/g | mg/g |
| Formulation | Compound I | Compound I | Compound I |
| Formulation 1 | 11.3 | 11.5 | 11.2 |
| Formulation 2 | 11.7 | 11.7 | 11.5 |
| Formulation 1 (+5% | 11.0 | 11.1 | 11.1 |
| water) | | | |
| Formulation 2 (+5% | 11.3 | 11.4 | 11.1 |
| water) | | | |

### Example 8: A Study to Evaluate the Effect of Compound I on Cognitive Function in Participants with Huntington's Disease

A randomized, placebo-controlled, double-blind study to evaluate the cognitive effects, safety, tolerability, and PK of Compound I in participants with premanifest or early manifest HD is conducted.

Approximately 178 participants will be randomized in the study to obtain 142 evaluable participants. Additional participants may be randomized if the early discontinuation rate is higher than expected. Participants will be adults with genetically confirmed expansion of the huntingtin gene-cytosine-adenine-guanine (CAG) trinucleotide repeat at screening who meet diagnostic criteria detailed in the inclusion criteria below (including Unified Huntington's Disease Rating Scale (UHDRS) scores and CAG-Age-Product [CAP]) scores within specific ranges).

1.2 mg Compound I or placebo will be provided as oral softgel lipid capsule for self-administration once daily in the morning through the day prior to Day 28 visit. Beginning at the Day 28 visit, 0.9 mg of Compound I or placebo will be provided as oral softgel lipid capsule for self-administration once daily in the morning for 56 days. Treatment with Compound 1 can be ended without down titration.

### Inclusion Criteria:

Meet all the following criteria for HD: (a) Genetically confirmed disease with CAG expansion ≥36; (b) UHDRS-TFC score >6 and <13; (c) No features of juvenile HD (i.e., UHDRS Diagnostic Confidence Level (DCL)=4 before age 25).
CAP score >70, as calculated using the CAP formula: AGE×(CAG−30)/6.49. Score <26 on the Montreal Cognitive Assessment (MoCA) at Screening.

### Example 9: Study to Evaluate the Effect of Compound I on Functioning Capacity in Participants with Huntington's Disease

A randomized, placebo-controlled, double-blind, parallel group study with a 28-day treatment duration to evaluate the effects of Compound I on functioning capacity, safety, and tolerability in participants with early manifest HD is conducted. Participants will be adults with genetically-confirmed expansion of the Huntingtin (HTT) gene cytosine, adenine, and guanine (CAG) trinucleotide repeat at Screening who meet diagnostic criteria detailed in the inclusion criteria below [including Unified Huntington's Disease Rating Scale (UHDRS) scores and CAG-Age-Product [CAP] scores within specific ranges]. Objectives of the study include: (1) assessing the magnitude of the baseline difference between participants with early Huntington's Disease (HD) and healthy participants (HP) with respect to measures of cognitive performance; (2) evaluating the effect of Compound I on cognition and functioning outcomes in participants with HD; (3) evaluating safety and tolerability of Compound I in participants with HD: (4) evaluating reliability and intercorrelations across measures of cognitive and functional status; (5) evaluating the pharmacokinetics (PK) of Compound I in participants with HD.

Eligible participants with HD will be randomized 1:1 to receive 1.2 mg of Compound I or placebo oral softgel lipid capsule for up to 28 days. Beginning on Day 1 and continuing through Day 28, participants with HD will self-administer blinded investigational product (IP) (i.e., 1.2 mg of Compound I or placebo) once per day in the morning. At clinic visits, participants with HD will take the IP under staff supervision, followed by assessments of cognitive function, proxy measures of real-world function, and patient and clinician/caregiver-rated measures of function. Study staff will dispense sufficient IP for daily administration until the next scheduled study visit. Adherence to the dosing regimen will be assessed at each in-clinic visit.

### Inclusion Criteria

Genetically confirmed disease with CAG expansion ≥36. UHDRS TFC>6 and <13, suggesting no more than a moderate level of functional impairment. No features of juvenile HD (i.e., UHDRS Diagnostic Confidence Level (DCL)=4 before age 25). CAP score >70, as calculated using the CAP formula: AGE×(CAG−30)/6.49. Score <26 on the Montreal Cognitive Assessment (MoCA) at Screening, indicating the presence of cognitive impairment.

### Example 10: A Study to Evaluate the Long-term Safety and Tolerability of Compound I in Participants with Huntington's Disease

A long-term, open-label study to evaluate the safety and tolerability of Compound I in rollover (see Examples 8 and 9) or de novo participants with premanifest or early manifest HD is conducted. Objectives of the study include: (1) evaluation of the safety and tolerability of Compound I softgel lipid capsule in participants with Huntington's Disease (HD); (2) evaluation of the effect of Compound I on HD-specific measures of daily functioning in participants with HD; (3) evaluation of the effect of Compound I on health-related quality of life and functioning in participants with HD; (4) evaluation of the effect of Compound I on cognitive performance in participants with HD; (5) determine the plasma pharmacokinetics (PK) of Compound I following daily administration of Compound I.

Beginning on Day 1 and continuing through Day 365, participants will self-administer investigational product (IP) (0.9 mg of Compound I softgel lipid capsule) once per day in the morning and will track IP intake in a participant diary. At clinic visits, participants will take the IP under staff supervision, followed by assessments of cognitive function, health-related function and quality of life, and neuropsychiatric symptoms (Day 1 assessments should be done predose). Study staff will dispense sufficient IP for daily administration until the next scheduled study visit. Adherence to the dosing regimen will be assessed at each in-clinic visit Treatment with Compound I can be discontinued without down titration.

### Inclusion Criteria:

Completed previous HD clinical trial. Meet all of the following criteria for HD: (a) Genetically confirmed disease with CAG expansion ≥40; (b) No features of juvenile HD (i.e., UHDRS Diagnostic Confidence Level=4 before age 25 years). CAG-Age-Product (CAP) score ≥90, as calculated using the CAP formula: AGE×(CAG−30)/6.49. UHDRS-TFC=13 or MoCA>25 score at Screening.

### Example 11: A Study to Evaluate the Effects of Compound I in Participants with Mild Cognitive Impairment or Mild Dementia Due to Alzheimer's Disease

A randomized, double-blind, placebo-controlled study to evaluate the effects of Compound I in participants with mild cognitive impairment (MCI) or mild dementia due to AD is conducted. Objectives of the study include: (1) evaluating the effect of Compound I on cognitive performance in participants with Alzheimer's Disease (AD); (2) evaluating the safety and tolerability of Compound I softgel lipid capsule in participants with AD; (3) evaluating the safety and tolerability of Compound I softgel lipid capsule on other safety parameters; (4) evaluating the effect of Compound I on additional measures of global and domain-specific cognitive performance in participants with AD; (5) evaluating the effect of Compound I on global functioning in participants with AD; (6) evaluating the effect of Compound I on neuropsychiatric functioning in participants with AD; (7) evaluating pharmacokinetics (PK) exposure-response; (8) evaluating relationship of exploratory peripheral biomarkers with other endpoints.

Eligible participants with a confirmed diagnosis and who meet the criteria for MCI or Mild Dementia due to AD at Screening will be randomized to receive either Compound I or matching placebo. Within the Compound I treatment arm, participants will receive 1.2 mg of Compound I as a softgel lipid capsule orally once daily for the first 6 weeks (Days 1 to 42 [±2 days]), followed by 0.9 mg of Compound I as a softgel lipid capsule orally once daily for 6 weeks. The placebo arm will receive placebo throughout the Treatment Period (12 weeks). All participants will self-administer investigational product (IP) in the morning. No dosing will be permitted beyond Day 84.

### Inclusion Criteria:

Meet the following criteria for MCI or mild dementia due to AD at Screening: (a) a memory complaint reported by the participant or their study partner; (b) a Clinical Dementia Rating (CDR) score of 0.5 to 1.0 (inclusive) with a memory box score ≥0.5; (c) essentially preserved activities of daily living, in the opinion of the investigator; (d) brain MRI report, obtained within the 2 years preceding the Baseline Period, that is consistent with the diagnosis of AD with no clinically significant findings of non-AD pathology that could account for the observed cognitive impairment. Have a score of 15 to 25 (inclusive) on the MoCA at Screening.

### Example 12: A Study to Evaluate the Effects of Compound I in Parkinson's Disease Cognitive Impairment

A randomized, placebo-controlled, double-blind study to evaluate the effects of Compound I in PD cognitive impairment is conducted. Objectives of the study include: (1) evaluating the effect of Compound I on cognitive performance in participants with Parkinson's Disease (PD)-Mild Cognitive Impairment (MCI); (2) evaluating the safety and tolerability of Compound I oral capsule in participants with PD-MCI; (3) evaluating the safety and tolerability of Compound I oral capsule on other safety parameters; (4) evaluating the effect of Compound I on motor symptoms in participants with PD-MCI; (5) evaluating the effect of Compound I on functioning in participants with PD-MCI; (6) evaluating the effect of Compound I on additional measures of global and domain-specific cognitive performance in participants with PD-MCI; (7) evaluating pharmacokinetics (PK) exposure-response.

Eligible participants with a confirmed diagnosis of idiopathic PD by 2015 Movement Disorder Society (MDS) criteria at Screening and who meet MDS Task Force Criteria for MCI in PD (excluding requirement for United Kingdom (UK) PD Brain Bank diagnostic criteria) will receive a 1.2 mg dose of Compound I or placebo daily for 6 weeks.

### Inclusion Criteria:

Meet the following criteria for PD-MCI: (a) have a confirmed diagnosis of idiopathic PD according to 2015 MDS clinical diagnostic criteria, and (b) meet MDS Task Force Criteria for MCI in PD (excluding requirement for UK PD Brain Bank diagnostic criteria). For participants meeting Level 1 PD-MCI criteria, have a MoCA score of 20 to 25 (inclusive) at Screening. For participants meeting Level 2 PD-MCI criteria (within the past year), have a MoCA score of 18 to 25 (inclusive) at Screening. Meet criteria for modified Hoehn and Yahr Stage I to III (mild to moderate motor severity) at Screening. Have stable motor symptoms for at least 4 weeks prior to Screening, in the opinion of the investigator.

### Example 13—Open-label, Single-dose Study to Evaluate the Pharmacokinetics, Safety, and Tolerability of Compound I

In an open-label, parallel group study to evaluate the PK, safety, and tolerability of Compound I, a single 1.2 mg oral dose of softgel capsules was administered to participants with hepatic impairment and in participants with normal hepatic function. Part 1 of the study comprised participants with moderate hepatic impairment (as determined by the Child-Pugh classifications; Class B (7 to 9 points)) and participants with normal hepatic function assigned to Cohorts 1 and 2, respectively. After Cohort 1 had been completely enrolled, participants with normal hepatic function (Cohort 2) were matched to Cohort 1 participants with respect to sex, mean age (10 years) and mean body mass index (BMI; 20%).

On Day 1, participants in Cohorts 1 and 2 received a single oral dose of 1.2 mg Compound I softgel capsules in the morning. Serial blood samples for PK analysis were collected through 168 hours post dose. Results of the study for Cohorts 1 and 2 are summarized in Tables 11 and 12, respectively.

TABLE 11
Summary of Cohort 1 PK Parameters

| | | | | AUCₗₐₛₜ | AUC_{inf} | | |
|---|---|---|---|---|---|---|---|
| | Cₘₐₓ | tₘₐₓ | tₗₐₛₜ | (ng*h/ | (ng*h/ | CL_{z}/F | V_{z}/F |
| Subject | (ng/mL) | (h) | (h) | mL) | mL) | (L/h) | (L) |
| N | 8 | 8 | 8 | 8 | 8 | 8 | 8 |
| GeoMean | 12.2 | 3.69 | 53.9 | 188 | 218 | 5.5 | 153 |
| CVGeoMean | 27.4 | 53.6 | 39.8 | 33.8 | 34.4 | 34.4 | 24.6 |

Cₘₐₓ: maximum concentration; tₘₐₓ: time of maximum concentration; tₗₐₛₜ: time of last measurable concentration; AUCₗₐₛₜ: area under the concentration vs. time curve from time 0 to tₗₐₛₜ; AUC_{inf}: area under the concentration vs. time curve from time 0 to infinite time; CLz/F: apparent clearance; Vz/F: apparent volume of distribution; N: number of values used to calculate summary statistics; GeoMean: geometric mean; CVGeoMean: coefficient of variation of geometric mean (%).

TABLE 12
Summary of Cohort 2 PK Parameters

| | | | | AUCₗₐₛₜ | AUC_{inf} | | |
|---|---|---|---|---|---|---|---|
| | Cₘₐₓ | tₘₐₓ | tₗₐₛₜ | (ng*h/ | (ng*h/ | CL_{z}/F | V_{z}/F |
| Subject | (ng/mL) | (h) | (h) | mL) | mL) | (L/h) | (L) |
| N | 8 | 8 | 8 | 7 | 7 | 7 | 7 |
| GeoMean | 15.7 | 3.99 | 64.3 | 238 | 255 | 4.71 | 151 |
| CVGeoMean | 31.4 | 27.6 | 65.1 | 50.9 | 43.8 | 43.8 | 16.1 |

Cₘₐₓ: maximum concentration; tₘₐₓ: time of maximum concentration; tₗₐₛₜ: time of last measurable concentration; AUCₗₐₛₜ: area under the concentration vs. time curve from time 0 to tₗₐₛₜ; AUC_{inf}: area under the concentration vs. time curve from time 0 to infinite time; CLz/F: apparent clearance; Vz/F: apparent volume of distribution; N: number of values used to calculate summary statistics; GeoMean: geometric mean; CVGeoMean: coefficient of variation of geometric mean (%).

### NUMBERED EMBODIMENTS

Particular embodiments of the disclosure are set forth in the following numbered paragraphs:
1. A pharmaceutical composition comprising
   a) Compound I:

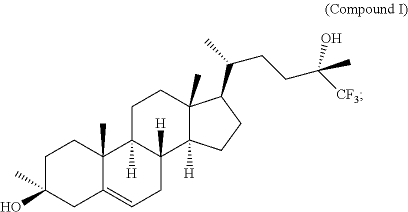

or a pharmaceutically acceptable salt thereof; and
b) one or more glycerides, wherein said glyceride comprises between about 30% and about 100% w/w monoglycerides.
2. The pharmaceutical composition according to embodiment 1, wherein the pharmaceutical composition comprises Compound I.
3. The pharmaceutical composition according to any one of embodiments 1-2, wherein the pharmaceutical composition comprises a pharmaceutically acceptable salt of Compound I.
4. The pharmaceutical composition according to any one of embodiments 1-3, wherein the pharmaceutical composition comprises about 0.2% to about 0.4% w/w Compound I.
5. The pharmaceutical composition according to embodiment 4, wherein the pharmaceutical composition comprises about 0.25% to about 0.35% w/w Compound I.
6. The pharmaceutical composition according to embodiment 5, wherein the pharmaceutical composition comprises about 0.3% w/w Compound I.
7. The pharmaceutical composition according to embodiment 6, wherein the pharmaceutical composition comprises 0.3% w/w Compound I.
8. The pharmaceutical composition according to any one of embodiments 1-3, wherein the pharmaceutical composition comprises about 3 mg/g of Compound I.
9. The pharmaceutical composition according to any one of embodiments 1-3, wherein the pharmaceutical composition comprises 3 mg/g of Compound I.
10. The pharmaceutical composition according to any one of embodiments 1-9, wherein the one or more glycerides is selected from the group consisting of glyceryl mono-/di-caprylocaprate, glyceryl mono-/di-caprylocaprate, glyceryl monocaprylate Type 1, glyceryl monocaprylate Type 1, glyceryl monocaprylocaprate Type 1, glyceryl monolinoleate, and combinations thereof.
11. The pharmaceutical composition according to any one of embodiments 1-9, wherein the one or more glycerides is selected from the group consisting of glyceryl monocaprylate, glyceryl monolinoleate and a combination thereof.
12. The pharmaceutical composition according to any one of embodiments 1-11, wherein the pharmaceutical composition comprises glyceryl monocaprylate.
13. The pharmaceutical composition according to any one of embodiments 1-11, wherein the pharmaceutical composition comprises glyceryl monolinoleate.
14. The pharmaceutical composition according to any one of embodiments 1-11, wherein the pharmaceutical composition comprises glyceryl monocaprylate and glyceryl monolinoleate.
15. The pharmaceutical composition according to any one of embodiments 1-14, wherein the amount of the one or more glycerides is from about 20% w/w to about 80% w/w.
16. The pharmaceutical composition according to embodiment 15, wherein the amount of the one or more glycerides is from about 20% w/w to about 60% w/w.
17. The pharmaceutical composition according to any one of embodiment 11, 12 or 14, wherein the amount of glyceryl monocaprylate in the composition is from about 50% w/w to about 80% w/w.
18. The pharmaceutical composition according to embodiment 17, wherein the amount of glyceryl monocaprylate is from about 55% w/w to about 60% w/w.
19. The pharmaceutical composition according to embodiment 18, wherein the amount of glyceryl monocaprylate is about 50% w/w.
20. The pharmaceutical composition according to embodiment 18, wherein the amount of glyceryl monocaprylate is 50% w/w.
21. The pharmaceutical composition according to embodiment 18, wherein the amount of glyceryl monocaprylate is about 60% w/w.
22. The pharmaceutical composition according to embodiment 18, wherein the amount of glyceryl monocaprylate is 60% w/w.
23. The pharmaceutical composition according to any one of embodiments 11, 13 or 14, wherein the amount of glyceryl monolinoleate is from about 20% w/w to about 30% w/w.
24. The pharmaceutical composition according to embodiment 23, wherein the amount of glyceryl monolinoleate is about 20% w/w.
25. The pharmaceutical composition according to embodiment 23, wherein the amount of glyceryl monolinoleate is 20% w/w.
26. The pharmaceutical composition according to embodiment 23, wherein the amount of glyceryl monolinoleate is about 22.5% w/w.
27. The pharmaceutical composition according to embodiment 23, wherein the amount of glyceryl monolinoleate is 22.5% w/w.
28. The pharmaceutical composition according to any one of embodiments 1-27, further comprising stearoyl polyoxyl-32 glyceride.
29. The pharmaceutical composition according to embodiment 28, wherein the amount of stearoyl polyoxyl-32 glyceride is from about 5% w/w to about 20% w/w.
30. The pharmaceutical composition according to embodiment 28, wherein the amount of stearoyl polyoxyl-32 glyceride is from about 10% w/w to about 20% w/w.
31. The pharmaceutical composition according to embodiment 29, wherein the amount of stearoyl polyoxyl-32 glyceride is about 7.5% w/w.
32. The pharmaceutical composition according to embodiment 29, wherein the amount of stearoyl polyoxyl-32 glyceride is 7.5% w/w.
33. The pharmaceutical composition according to embodiment 30, wherein the amount of stearoyl polyoxyl-32 glyceride is about 10% w/w.
34. The pharmaceutical composition according to embodiment 30, wherein the amount of stearoyl polyoxyl-32 glyceride is 10% w/w.
35. The pharmaceutical composition according to embodiment 29 or 30, wherein the amount of stearoyl polyoxyl-32 glyceride) is about 20% w/w.
36. The pharmaceutical composition according to embodiment 29 or 30, wherein the amount of stearoyl polyoxyl-32 glyceride is 20% w/w.
37. The pharmaceutical composition according to any one of embodiments 1-36, wherein the composition further comprises a surfactant.
38. The pharmaceutical composition according to embodiment 37, wherein the amount of surfactant is from about 10% w/w to about 25% w/w.
39. The pharmaceutical composition according to embodiment 38, wherein the amount of surfactant is about 10% w/w.
40. The pharmaceutical composition according to embodiment 38, wherein the amount of surfactant is 10% w/w.
41. The pharmaceutical composition according to embodiment 38, wherein the amount of surfactant is about 20% w/w.
42. The pharmaceutical composition according to embodiment 38, wherein the amount of surfactant is 20% w/w.
43. The pharmaceutical composition according to any one of embodiment 38-42, wherein the surfactant is vitamin E TPGS.
44. The pharmaceutical composition according to any one of embodiments 1-43, wherein the composition further comprises an antioxidant.
45. The pharmaceutical composition according to embodiment 44, wherein the amount of antioxidant is from about 0.005% w/w to about 0.02% w/w.
46. The pharmaceutical composition according to embodiment 45, wherein the amount of antioxidant is about 0.005% w/w.
47. The pharmaceutical composition according to embodiment 45, wherein the amount of antioxidant is 0.005% w/w.
48. The pharmaceutical composition according to embodiment 45, wherein the amount of antioxidant is about 0.01% w/w.
49. The pharmaceutical composition according to embodiment 45, wherein the amount of antioxidant is 0.01% w/w.
50. The pharmaceutical composition according to embodiment 45, wherein the amount of antioxidant is about 0.02% w/w.
51. The pharmaceutical composition according to embodiment 45, wherein the amount of antioxidant is 0.02% w/w.
52. The pharmaceutical composition according to any one of embodiments 44-51, wherein the antioxidant is butylated hydroxyanisole (BHA).
53. A pharmaceutical composition comprising:
   a) about 0.3% w/w Compound I,
   b) about 60% w/w glyceryl monocaprylate,
   c) about 20% w/w glyceryl monolinoleate,
   d) about 20% w/w vitamin E TPGS, and
   e) about 0.01% w/w butylated hydroxyanisole.
54. A pharmaceutical composition comprising:
   a) about 0.3% w/w Compound I,
   b) about 50% w/w glyceryl monocaprylate,
   c) about 22.5% w/w glyceryl monolinoleate,
   d) about 20% w/w vitamin E TPGS,
   e) about 7.5% w/w stearoyl polyoxyl-32 glyceride, and
   f) about 0.01% w/w butylated hydroxyanisole.
55. The pharmaceutical composition of any one of embodiments 1-54, wherein the pharmaceutical composition is an oral dosage form.
56. The pharmaceutical composition of embodiment 55, wherein the pharmaceutical composition is a lipid formulation.
57. The pharmaceutical composition of embodiment 55, wherein the oral dosage form is a soft gelatin capsule, a hardshell capsule or an oral liquid.
58. The pharmaceutical composition according to any one of embodiments 1-57, wherein following administration of the pharmaceutical composition to a subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
59. A soft gelatin capsule comprising
   a) Compound I.

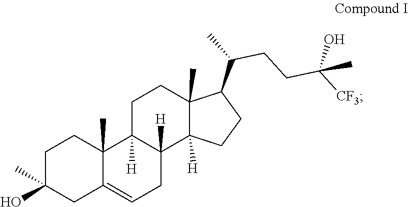

or a pharmaceutically acceptable salt thereof; and
   b) one or more glycerides, wherein said glycerides comprise between about 30% and about 100% w/w monoglycerides.
60. The soft gelatin capsule of embodiment 59, wherein the soft gelatin capsule comprises Compound I.
61. The soft gelatin capsule of embodiment 59, wherein the soft gelatin capsule comprises a pharmaceutically acceptable salt of Compound I.
62. The soft gelatin capsule of any one of embodiments 59-61, wherein the soft gelatin capsule comprises about 0.2% to about 0.4% w/w Compound I.
63. The soft gelatin capsule of embodiment 62, wherein the soft gelatin capsule comprises about 0.25% to about 0.35% w/w Compound I.
64. The soft gelatin capsule of embodiment 62, wherein the soft gelatin capsule comprises about 0.3% w/w Compound I.
65. The soft gelatin capsule of embodiment 62, wherein the soft gelatin capsule comprises 0.3% w/w Compound I.
66. The soft gelatin capsule of embodiment 59, wherein the soft gelatin capsule comprises about 3 mg/g of Compound I.
67. The soft gelatin capsule of embodiment 59, wherein the soft gelatin capsule comprises about 3 mg/g of Compound I.
68. The soft gelatin capsule of any one of embodiments 59-67, wherein the one or more glycerides is selected from the group consisting of glyceryl mono-/di-caprylocaprate, glyceryl mono-/di-caprylocaprate, glyceryl monocaprylate Type 1, glyceryl monocaprylate Type 1, glyceryl monocaprylocaprate Type 1, glyceryl monolinoleate, and combinations thereof.
69. The soft gelatin capsule of any one of embodiments 59-68, wherein one or more glycerides is selected from the group consisting of glyceryl monocaprylate, glyceryl monolinoleate and a combination thereof.
70. The soft gelatin capsule of any one of embodiments 59-69, wherein the soft gelatin capsule comprises glyceryl monocaprylate.
71. The soft gelatin capsule of any one of embodiments 59-69, wherein the soft gelatin capsule comprises glyceryl monolinoleate.
72. The soft gelatin capsule of any one of embodiments 59-69, wherein the soft gelatin capsule comprises glyceryl monocaprylate and glyceryl monolinoleate.
73. The soft gelatin capsule of any one of embodiments 59-72, wherein the amount of the one or more glycerides is from about 20% w/w to about 80% w/w.
74. The soft gelatin capsule according to embodiment 73, wherein the amount of the one or more glycerides is from about 20% w/w to about 60% w/w.
75. The soft gelatin capsule according to any one of embodiment 69, 70 or 72, wherein the amount of glyceryl monocaprylate is from about 50% w/w to about 80% w/w.
76. The soft gelatin capsule according to embodiment 75, wherein the amount of glyceryl monocaprylate is from about 55% w/w to about 60% w/w.
77. The soft gelatin capsule according to embodiment 75, wherein the amount of glyceryl monocaprylate is about 50% w/w.
78. The soft gelatin capsule according to embodiment 75, wherein the amount of glyceryl monocaprylate is 50% w/w.
79. The soft gelatin capsule according to embodiment 75, wherein the amount of glyceryl monocaprylate is about 60% w/w.
80. The soft gelatin capsule according to embodiment 75, wherein the amount of glyceryl monocaprylate is 60% w/w.
81. The soft gelatin capsule according to any one of embodiments 69, 71-72, wherein the amount of glyceryl monolinoleate is from about 20% w/w to about 30% w/w.
82. The soft gelatin capsule according to embodiment 81, wherein the amount of glyceryl monolinoleate is about 20% w/w.
83. The soft gelatin capsule according to embodiment 81, wherein the amount of glyceryl monolinoleate is 20% w/w.
84. The soft gelatin capsule according to embodiment 81, wherein the amount of glyceryl monolinoleate is about 22.5% w/w.
85. The soft gelatin capsule according to embodiment 81, wherein the amount of glyceryl monolinoleate is 22.5% w/w.
86. The soft gelatin capsule according to any one of embodiments 59-85, further comprising stearoyl polyoxyl-32 glyceride.
87. The soft gelatin capsule according to embodiment 86, wherein the amount of stearoyl polyoxyl-32 glyceride is from about 5% w/w to about 20% w/w.
88. The soft gelatin capsule according to embodiment 86, wherein the amount of stearoyl polyoxyl-32 glyceride is from about 10% w/w/to about 20% w/w.
89. The soft gelatin capsule according to embodiment 87, wherein the amount of stearoyl polyoxyl-32 glyceride is about 7.5% w/w.
90. The soft gelatin capsule according to embodiment 87, wherein the amount of stearoyl polyoxyl-32 glyceride is 7.5% w/w.
91. The soft gelatin capsule according to embodiment 87 or 88, wherein the amount of stearoyl polyoxyl-32 glyceride is about 10% w/w.
92. The soft gelatin capsule according to embodiment 87 or 88, wherein the amount of stearoyl polyoxyl-32 glyceride is 10% w/w.
93. The soft gelatin capsule according to embodiment 87 or 88, wherein the amount of stearoyl polyoxyl-32 glyceride is about 20% w/w.
94. The soft gelatin capsule according to embodiment 87 or 88, wherein the amount of stearoyl polyoxyl-32 glyceride is 20% w/w.
95. The soft gelatin capsule according to any one of embodiments 59-94, further comprising a surfactant.
96. The soft gelatin capsule according to embodiment 95, wherein the amount of surfactant is from about 10% w/w to about 25% w/w.
97. The soft gelatin capsule according to embodiment 96, wherein the amount of surfactant is about 10% w/w.
98. The soft gelatin capsule according to embodiment 96, wherein the amount of surfactant is 10% w/w.
99. The soft gelatin capsule according to embodiment 96, wherein the amount of surfactant is about 20% w/w.
100. The soft gelatin capsule according to embodiment 96, wherein the amount of surfactant is 20% w/w.
101. The soft gelatin capsule according to any one of embodiment 95-100, wherein the surfactant is vitamin E TPGS.
102. The soft gelatin capsule according to any one of embodiments 59-101, further comprising an antioxidant.
103. The soft gelatin capsule according to embodiment 102, wherein the amount of antioxidant is from about 0.005% w/w to about 0.02% w/w.
104. The soft gelatin capsule according to embodiment 103, wherein the amount of antioxidant is about 0.005% w/w.
105. The soft gelatin capsule according to embodiment 103, wherein the amount of antioxidant is 0.005% w/w.
106. The soft gelatin capsule according to embodiment 103, wherein the amount of antioxidant is about 0.01% w/w.
107. The soft gelatin capsule according to embodiment 103, wherein the amount of antioxidant is 0.01% w/w.
108. The soft gelatin capsule according to embodiment 103, wherein the amount of antioxidant is about 0.02% w/w.
109. The soft gelatin capsule according to embodiment 103 wherein the amount of antioxidant is 0.02% w/w.
110. The soft gelatin capsule according to any one of embodiments 102-109 wherein the antioxidant is butylated hydroxyanisole (BHA).
111. A soft gelatin capsule comprising:
   a) about 0.3% w/w Compound I,
   b) about 60% w/w glyceryl monocaprylate,
   c) about 20% w/w glyceryl monolinoleate,
   d) about 20% w/w vitamin E TPGS, and
   e) about 0.01% w/w butylated hydroxyanisole.
112. A soft gelatin capsule comprising:
   a) about 0.3% w/w Compound I,
   b) about 50% w/w glyceryl monocaprylate,
   c) about 22.5% w/w glyceryl monolinoleate,
   d) about 20% w/w vitamin E TPGS,
   e) about 7.5% w/w stearoyl polyoxyl-32 glyceride, and
   f) about 0.01% w/w butylated hydroxyanisole.
113. A pharmaceutical composition comprising a therapeutically effective amount of Compound I

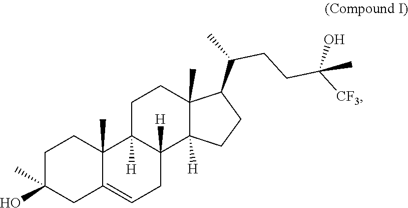

or a pharmaceutically acceptable salt thereof;
and a pharmaceutically acceptable carrier;
wherein following administration of the pharmaceutical composition to a subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%,
114. The pharmaceutical composition according to embodiment 113, wherein the pharmaceutical composition provides a Cₘₐₓ of between about 8 and about 25 ng/mL.
115. The pharmaceutical composition according to embodiment 114, wherein the pharmaceutical composition provides a Cₘₐₓ of about 15.7 ng/mL.
116. The pharmaceutical composition according one of embodiments 113-115, wherein the pharmaceutical composition provides a tₘₐₓ of between about 2 and about 8 hours.
117. The pharmaceutical composition according to embodiment 116, wherein the pharmaceutical composition provides a tₘₐₓ of about 4 hours.
118. The pharmaceutical composition according to any one of embodiments 113-117, wherein the pharmaceutical composition provides an AUCₗₐₛₜ of between about 100 and about 450 ng*h/mL.
119. The pharmaceutical composition according to embodiment 118, wherein the pharmaceutical composition provides an AUCₗₐₛₜ of about 238 ng*h/mL.
120. The pharmaceutical composition according to any one of embodiments 113-119, wherein the pharmaceutical composition provides an AUC_{inf} of between about 100 and about 500 ng*h/mL.
121. The pharmaceutical composition according to embodiment 120, wherein the pharmaceutical composition provides an AUC_{inf} of about 255 ng*h/mL.
122. The pharmaceutical composition according to any one of embodiments 113-121, wherein the pharmaceutical composition provides a V_{z}/F of between about 100 and about 250 L.
123. The pharmaceutical composition according to embodiment 122, wherein the pharmaceutical composition provides a V_{z}/F of about 151 L.
124. The pharmaceutical composition according to any one of embodiments 113-123, wherein the pharmaceutical composition provides a CL_{z}/F of between about 2 and about 12 L/h.
125. The pharmaceutical composition according to embodiment 124, wherein the pharmaceutical composition provides a CL_{z}/F of about 4.7 L/h.
126. The pharmaceutical composition according to any one of embodiments 113-125, wherein the pharmaceutical composition provides a bioavailability of between about 70% to about 100%.
127. The pharmaceutical composition according to embodiment 126, wherein the pharmaceutical composition provides a bioavailability of between about 95% to about 100%.
128. The pharmaceutical composition according to embodiment 127, wherein the pharmaceutical composition provides a bioavailability of about 100%.
129. The pharmaceutical composition according to any one of embodiments 113-128, wherein said pharmacokinetic parameter is obtained following a single administration of the pharmaceutical composition.
130. The pharmaceutical composition according to any one of embodiments 113-128, wherein said pharmacokinetic parameter is obtained following 2-60 administrations of the pharmaceutical composition.
131. The pharmaceutical composition according to any one of embodiments 113-130, wherein said pharmacokinetic parameter is obtained under fasted conditions.
132. The pharmaceutical composition according to any one of embodiments 113-130, wherein said pharmacokinetic parameter is obtained under fed conditions.
133. The pharmaceutical composition according to embodiment 132, wherein said pharmacokinetic parameter is obtained under high fat fed conditions.
134. The pharmaceutical composition according to embodiment 132, wherein said pharmacokinetic parameter is obtained under low fat fed conditions.
135. The pharmaceutical composition according to any one of embodiments 113-134, wherein the pharmaceutical composition is an oral dosage form.
136. The pharmaceutical composition according to any one of embodiments 113-135, wherein the pharmaceutical composition is a lipid formulation.
137. The pharmaceutical composition according to embodiment 136, wherein the oral dosage form is a soft gelatin capsule, a hardshell capsule or an oral liquid.
138. The pharmaceutical composition according to any one of embodiments 113-137, comprising one or more glycerides, wherein said glyceride comprises between about 30% and about 100% w/w monoglycerides.
139. The pharmaceutical composition according to any one of embodiments 113-138, wherein said pharmaceutical composition comprises:
   a) about 0.3% w/w Compound I,
   b) about 60% w/w glyceryl monocaprylate,
   c) about 20% w/w glyceryl monolinoleate,
   d) about 20% w/w vitamin E TPGS, and
   e) about 0.01% w/w butylated hydroxyanisole.
140. The pharmaceutical composition according to any one of embodiments 113-138, wherein said pharmaceutical composition comprises:
   a) about 0.3% w/w Compound I,
   b) about 50% w/w glyceryl monocaprylate,
   c) about 22.5% w/w glyceryl monolinoleate,
   d) about 20% w/w vitamin E TPGS,
   e) about 7.5% w/w stearoyl polyoxyl-32 glyceride, and
   f) about 0.01% w/w butylated hydroxyanisole.
141. A method of treating Huntington's disease in a subject, comprising administering to the subject a therapeutically effective amount of a pharmaceutical composition according to any one of embodiments 1-58 or 113-140.
142. A method of treating cognitive impairment in a subject having Huntington's disease, comprising administering to the subject a therapeutically effective amount of a pharmaceutical composition according to any one of embodiments 1-58 or 113-140.
143. A method of treating Mild Cognitive Impairment (MCI) associated with Huntington's disease in a subject, comprising administering to the subject a therapeutically effective amount of a pharmaceutical composition according to any one of embodiments 1-58 or 113-140.
144. A method of improving executive function in a subject having Mild Cognitive Impairment (MCI) associated with Huntington's disease, comprising administering to the subject a therapeutically effective amount of a pharmaceutical composition according to any one of embodiments 1-58 or 113-140.
145. A method of improving working memory in a subject having Mild Cognitive Impairment (MCI) associated with Huntington's disease, comprising administering to the subject a therapeutically effective amount of a pharmaceutical composition according to any one of embodiments 1-58 or 113-140.
146. A method of improving learning in a subject having Mild Cognitive Impairment (MCI) associated with Huntington's disease, comprising administering to the subject a therapeutically effective amount of a pharmaceutical composition according to any one of embodiments 1-58 or 113-140.
147. A method of improving learning and working memory in a subject having Mild Cognitive Impairment (MCI) associated with Huntington's disease, comprising administering to the subject a therapeutically effective amount of a pharmaceutical composition according to any one of embodiments 1-58 or 113-140.
148. A method of improving cognition in a subject having Huntington's disease, comprising administering to the subject a therapeutically effective amount of a pharmaceutical composition according to any one of embodiments 1-58 or 113-140.
149. A method for slowing cognitive decline in a subject having Huntington's disease, comprising administering to the subject a therapeutically effective amount of a pharmaceutical composition according to any one of embodiments 1-58 or 113-140.
150. A method of treating Parkinson's disease in a subject, comprising administering to the subject a therapeutically effective amount of a pharmaceutical composition according to any one of embodiments 1-58 or 113-140.
151. A method of treating cognitive impairment in a subject having Parkinson's disease, comprising administering to the subject a therapeutically effective amount of a pharmaceutical composition according to any one of embodiments 1-58 or 113-140.
152. A method of treating Mild Cognitive Impairment (MCI) associated with Parkinson's disease in a subject, comprising administering to the subject a therapeutically effective amount of a pharmaceutical composition according to any one of embodiments 1-58 or 113-140.
153. A method of improving executive function in a subject having Mild Cognitive Impairment (MCI) associated with Parkinson's disease, comprising administering to the subject a therapeutically effective amount of a pharmaceutical composition according to any one of embodiments 1-58 or 113-140.
154. A method of improving working memory in a subject having Mild Cognitive Impairment (MCI) associated with Parkinson's disease, comprising administering to the subject a therapeutically effective amount of a pharmaceutical composition according to any one of embodiments 1-58 or 113-140.
155. A method of improving learning in a subject having Mild Cognitive Impairment (MCI) associated with Parkinson's disease, comprising administering to the subject a therapeutically effective amount of a pharmaceutical composition according to any one of embodiments 1-58 or 113-140.
156. A method of improving learning and working memory in a subject having Mild Cognitive Impairment (MCI) associated with Parkinson's disease, comprising administering to the subject a therapeutically effective amount of a pharmaceutical composition according to any one of embodiments 1-58 or 113-140.
157. A method of improving cognition in a subject having Parkinson's disease, comprising administering to the subject a therapeutically effective amount of a pharmaceutical composition according to any one of embodiments 1-58 or 113-140.
158. A method for slowing cognitive decline in a subject having Parkinson's disease, comprising administering to the subject a therapeutically effective amount of a pharmaceutical composition according to any one of embodiments 1-58 or 113-140.
159. A method of treating Alzheimer's disease in a subject, comprising administering to the subject a therapeutically effective amount of a pharmaceutical composition according to any one of embodiments 1-58 and 113-140.
160. A method of treating cognitive impairment in a subject having Alzheimer's disease, comprising administering to the subject a therapeutically effective amount of a pharmaceutical composition according to any one of embodiments 1-58 and 113-140.
161. A method of treating Mild Cognitive Impairment (MCI) associated with Alzheimer's disease in a subject, comprising administering to the subject a therapeutically effective amount of a pharmaceutical composition according to any one of embodiments 1-58 and 113-140.
162. A method of improving executive function in a subject having Mild Cognitive Impairment (MCI) associated with Alzheimer's disease, comprising administering to the subject a therapeutically effective amount of a pharmaceutical composition according to any one of embodiments 1-58 and 113-140.
163. A method of improving working memory in a subject having Mild Cognitive Impairment (MCI) associated with Alzheimer's disease, comprising administering to the subject a therapeutically effective amount of a pharmaceutical composition according to any one of embodiments 1-58 and 113-140.
164. A method of improving learning in a subject having Mild Cognitive Impairment (MCI) associated with Alzheimer's disease, comprising administering to the subject a therapeutically effective amount of a pharmaceutical composition according to any one of embodiments 1-58 and 113-140.
165. A method of improving learning and working memory in a subject having Mild Cognitive Impairment (MCI) associated with Alzheimer's disease, comprising administering to the subject a therapeutically effective amount of a pharmaceutical composition according to any one of embodiments 1-58 and 113-140.
166. A method of improving cognition in a subject having Alzheimer's disease, comprising administering to the subject a therapeutically effective amount of a pharmaceutical composition according to any one of embodiments 1-58 and 113-140.
167. A method for slowing cognitive decline in a subject having Alzheimer's disease, comprising administering to the subject a therapeutically effective amount of a pharmaceutical composition according to any one of embodiments 1-58 and 113-140.
168. A method of treating mild dementia associated with Alzheimer's disease, comprising administering to the subject a therapeutically effective amount of a pharmaceutical composition according to any one of embodiments 1-58 and 113-140.
169. A method of improving executive function in a subject having mild dementia associated with Alzheimer's disease, comprising administering to the subject a therapeutically effective amount of a pharmaceutical composition according to any one of embodiments 1-58 and 113-140.
170. A method of improving working memory in a subject having mild dementia associated with Alzheimer's disease, comprising administering to the subject a therapeutically effective amount of a pharmaceutical composition according to any one of embodiments 1-58 and 113-140.
171. A method of improving learning in a subject having mild dementia associated with Alzheimer's disease, comprising administering to the subject a therapeutically effective amount of a pharmaceutical composition according to any one of embodiments 1-58 and 113-140.
172. A method of improving learning and working memory in a subject having mild dementia associated with Alzheimer's disease, comprising administering to the subject a therapeutically effective amount of a pharmaceutical composition according to any one of embodiments 1-58 and 113-140.
173. A method of treating Huntington's disease in a subject, comprising administering to the subject a therapeutically effective amount of a soft gelatin capsule according to any one of embodiments 59-112.
174. A method of treating cognitive impairment in a subject having Huntington's disease, comprising administering to the subject a therapeutically effective amount of a soft gelatin capsule according to any one of embodiments 59-112.
175. A method of treating Mild Cognitive Impairment (MCI) associated with Huntington's disease in a subject, comprising administering to the subject a therapeutically effective amount of a soft gelatin capsule according to any one of embodiments 59-112.
176. A method of improving executive function in a subject having Mild Cognitive Impairment (MCI) associated with Huntington's disease, comprising administering to the subject a therapeutically effective amount of a soft gelatin capsule according to any one of embodiments 59-112.
177. A method of improving working memory in a subject having Mild Cognitive Impairment (MCI) associated with Huntington's disease, comprising administering to the subject a therapeutically effective amount of a soft gelatin capsule according to any one of embodiments 59-112.
178. A method of improving learning in a subject having Mild Cognitive Impairment (MCI) associated with Huntington's disease, comprising administering to the subject a therapeutically effective amount of a soft gelatin capsule according to any one of embodiments 59-112.
179. A method of improving learning and working memory in a subject having Mild Cognitive Impairment (MCI) associated with Huntington's disease, comprising administering to the subject a therapeutically effective amount of a soft gelatin capsule according to any one of embodiments 59-112.
180. A method of improving cognition in a subject having Huntington's disease, comprising administering to the subject a therapeutically effective amount of a soft gelatin capsule according to any one of embodiments 59-112.
181. A method for slowing cognitive decline in a subject having Huntington's disease, comprising administering to the subject a therapeutically effective amount of a soft gelatin capsule according to any one of embodiments 59-112.
182. A method of treating Parkinson's disease in a subject, comprising administering to the subject a therapeutically effective amount of a soft gelatin capsule according to any one of embodiments 59-112.
183. A method of treating cognitive impairment in a subject having Parkinson's disease, comprising administering to the subject a therapeutically effective amount of a soft gelatin capsule according to any one of embodiments 59-112.
184. A method of treating Mild Cognitive Impairment (MCI) associated with Parkinson's disease in a subject, comprising administering to the subject a therapeutically effective amount of a soft gelatin capsule according to any one of embodiments 59-112.
185. A method of improving executive function in a subject having Mild Cognitive Impairment (MCI) associated with Parkinson's disease, comprising administering to the subject a therapeutically effective amount of a soft gelatin capsule according to any one of embodiments 59-112.
186. A method of improving working memory in a subject having Mild Cognitive Impairment (MCI) associated with Parkinson's disease, comprising administering to the subject a therapeutically effective amount of a soft gelatin capsule according to any one of embodiments 59-112.
187. A method of improving learning in a subject having Mild Cognitive Impairment (MCI) associated with Parkinson's disease, comprising administering to the subject a therapeutically effective amount of a soft gelatin capsule according to any one of embodiments 59-112.
188. A method of improving learning and working memory in a subject having Mild Cognitive Impairment (MCI) associated with Parkinson's disease, comprising administering to the subject a therapeutically effective amount of a soft gelatin capsule according to any one of embodiments 59-112.
189. A method of improving cognition in a subject having Parkinson's disease, comprising administering to the subject a therapeutically effective amount of a soft gelatin capsule according to any one of embodiments 59-112.
190. A method for slowing cognitive decline in a subject having Parkinson's disease, comprising administering to the subject a therapeutically effective amount of a soft gelatin capsule according to any one of embodiments 59-112.
191. A method of treating Alzheimer's disease in a subject, comprising administering to the subject a therapeutically effective amount of a soft gelatin capsule according to any one of embodiments 59-112.
192. A method of treating cognitive impairment in a subject having Alzheimer's disease, comprising administering to the subject a therapeutically effective amount of a soft gelatin capsule according to any one of embodiments 59-112.
193. A method of treating Mild Cognitive Impairment (MCI) associated with Alzheimer's disease in a subject, comprising administering to the subject a therapeutically effective amount of a soft gelatin capsule according to any one of embodiments 59-112.
194. A method of improving executive function in a subject having Mild Cognitive Impairment (MCI) associated with Alzheimer's disease, comprising administering to the subject a therapeutically effective amount of a soft gelatin capsule according to any one of embodiments 59-112.
195. A method of improving working memory in a subject having Mild Cognitive Impairment (MCI) associated with Alzheimer's disease, comprising administering to the subject a therapeutically effective amount of a soft gelatin capsule according to any one of embodiments 59-112.
196. A method of improving learning in a subject having Mild Cognitive Impairment (MCI) associated with Alzheimer's disease, comprising administering to the subject a therapeutically effective amount of a soft gelatin capsule according to any one of embodiments 59-112.
197. A method of improving learning and working memory in a subject having Mild Cognitive Impairment (MCI) associated with Alzheimer's disease, comprising administering to the subject a therapeutically effective amount of a soft gelatin capsule according to any one of embodiments 59-112.
198. A method of improving cognition in a subject having Alzheimer's disease, comprising administering to the subject a therapeutically effective amount of a soft gelatin capsule according to any one of embodiments 59-112.
199. A method for slowing cognitive decline in a subject having Alzheimer's disease, comprising administering to the subject a therapeutically effective amount of a soft gelatin capsule according to any one of embodiments 59-112.
200. A method of treating mild dementia associated with Alzheimer's disease, comprising administering to the subject a therapeutically effective amount of a soft gelatin capsule according to any one of embodiments 59-112.
201. A method of improving executive function in a subject having mild dementia associated with Alzheimer's disease, comprising administering to the subject a therapeutically effective amount of a soft gelatin capsule according to any one of embodiments 59-112.
202. A method of improving working memory in a subject having mild dementia associated with Alzheimer's disease, comprising administering to the subject a therapeutically effective amount of a soft gelatin capsule according to any one of embodiments 59-112.
203. A method of improving learning in a subject having mild dementia associated with Alzheimer's disease, comprising administering to the subject a therapeutically effective amount of a soft gelatin capsule according to any one of embodiments 59-112.
204. A method of improving learning and working memory in a subject having mild dementia associated with Alzheimer's disease, comprising administering to the subject a therapeutically effective amount of a soft gelatin capsule according to any one of embodiments 59-112.
205. The method according to any one of embodiments 141-204, comprising administering to the subject a total daily dose of from about 0.3 mg to about 1.5 mg of Compound I.
206. The method according to any one of embodiments 141-204, comprising administering to the subject a total daily dose of from about 0.6 mg to about 1.5 mg of Compound I.
207. The method according to any one of embodiments 141-204, comprising administering to the subject a total daily dose of from about 0.9 mg to about 1.2 mg of Compound I.
208. The method according to any one of embodiments 141-204, comprising administering to the subject a total daily dose of about 1.2 mg of Compound I.
209. The method according to any one of embodiments 141-204, comprising administering to the subject a total daily dose of 1.2 mg of Compound I.
210. The method according to any one of embodiments 141-204, comprising administering to the subject a total daily dose of about 0.9 mg of Compound I.
211. The method according to any one of embodiments 141-204, comprising administering to the subject a total daily dose of 0.9 mg of Compound I.
212. The method according to any one of embodiments 141-204, comprising administering to the subject a total daily dose of about 0.6 mg of Compound I.
213. The method according to any one of embodiments 141-204, comprising administering to the subject a total daily dose of 0.6 mg of Compound I.
214. The method according to any one of embodiments 141-204, comprising administering to the subject a total daily dose of about 0.3 mg of Compound I.
215. The method according to any one of embodiments 141-204, comprising administering to the subject a total daily dose of 0.3 mg of Compound I.
216. The method according to any one of embodiments 141-204, wherein the dose of Compound I is administered to the subject at a frequency of once daily, twice daily, three times daily or four times daily.
217. The method according to any one of embodiments 141-204, comprising administering to the subject at least one loading dose; and at least one maintenance dose.
218. The method according to embodiment 217, comprising administering to the subject the loading dose daily for at least 2, 4, 6, 8 or 10 weeks.
219. The method according to embodiment 217 or 218, comprising administering to the subject the loading dose daily for at least 2 weeks.
220. The method according to embodiment 217 or 218, comprising administering to the subject the loading dose daily for at least 4 weeks.
221. The method according to embodiment 217 or 218, comprising administering to the subject the loading dose daily for at least 6 weeks.
222. The method according to any one of embodiments 217 or 218, comprising administering to the subject the loading dose daily for at least 8 weeks.
223. The method according to embodiment 217 or 218, comprising administering to the subject the loading dose daily for at least 10 weeks.
224. The method according to any one of embodiments 217-223, wherein the loading dose is about 1.2 mg daily of Compound I.
225. The method according to any one of embodiments 217-223, wherein the loading dose is 1.2 mg daily of Compound I.
226. The method according to any one of embodiments 217-225, wherein the maintenance dose is administered daily.
227. The method according to any one of embodiments 217-226, wherein the maintenance dose is about 0.9 mg daily of Compound I.
228. The method according to any one of embodiments 217-226, wherein the maintenance dose is 0.9 mg daily of Compound I.
229. The method according to any one of embodiments 217-226, wherein the maintenance dose is about 0.6 mg daily of Compound I.
230. The method according to any one of embodiments 217-226, wherein the maintenance dose is 0.6 mg daily of Compound I.
231. A pharmaceutical composition according to any one of embodiments 1-58 or 113-140, for use in treating Huntington's disease in a subject.
232. A pharmaceutical composition according to any one of embodiments 1-58 or 113-140, for use in treating cognitive impairment in a subject having Huntington's disease.
233. A pharmaceutical composition according to any one of embodiments 1-58 or 113-140, for use in treating Mild Cognitive Impairment (MCI) associated with Huntington's disease in a subject.
234. A pharmaceutical composition according to any one of embodiments 1-58 or 113-140, for use in improving executive function in a subject having Mild Cognitive Impairment (MCI) associated with Huntington's disease.
235. A pharmaceutical composition according to any one of embodiments 1-58 or 113-140, for use in improving working memory in a subject having Mild Cognitive Impairment (MCI) associated with Huntington's disease.
236. A pharmaceutical composition according to any one of embodiments 1-58 or 113-140, for use in improving learning in a subject having Mild Cognitive Impairment (MCI) associated with Huntington's disease.
237. A pharmaceutical composition according to any one of embodiments 1-58 or 113-140, for use in improving learning and working memory in a subject having Mild Cognitive Impairment (MCI) associated with Huntington's disease.
238. A pharmaceutical composition according to any one of embodiments 1-58 or 113-140, for use in improving cognition in a subject having Huntington's disease.
239. A pharmaceutical composition according to any one of embodiments 1-58 or 113-140, for use in slowing cognitive decline in a subject having Huntington's disease.
240. A pharmaceutical composition according to any one of embodiments 1-58 or 113-140, for use in treating Parkinson's disease in a subject.
241. A pharmaceutical composition according to any one of embodiments 1-58 or 113-140, for use in treating cognitive impairment in a subject having Parkinson's disease.
242. A pharmaceutical composition according to any one of embodiments 1-58 or 113-140, for use in treating Mild Cognitive Impairment (MCI) associated with Parkinson's disease in a subject.
243. A pharmaceutical composition according to any one of embodiments 1-58 or 113-140, for use in improving executive function in a subject having Mild Cognitive Impairment (MCI) associated with Parkinson's disease.
244. A pharmaceutical composition according to any one of embodiments 1-58 or 113-140, for use in improving working memory in a subject having Mild Cognitive Impairment (MCI) associated with Parkinson's disease.
245. A pharmaceutical composition according to any one of embodiments 1-58 or 113-140, for use in improving learning in a subject having Mild Cognitive Impairment (MCI) associated with Parkinson's disease.
246. A pharmaceutical composition according to any one of embodiments 1-58 or 113-140, for use in improving learning and working memory in a subject having Mild Cognitive Impairment (MCI) associated with Parkinson's disease.
247. A pharmaceutical composition according to any one of embodiments 1-58 or 113-140, for use in improving cognition in a subject having Parkinson's disease.
248. A pharmaceutical composition according to any one of embodiments 1-58 or 113-140, for use in slowing cognitive decline in a subject having Parkinson's disease.
249. A pharmaceutical composition according to any one of embodiments 1-58 or 113-140, for use in treating Alzheimer's disease in a subject.
250. A pharmaceutical composition according to any one of embodiments 1-58 or 113-140, for use in treating cognitive impairment in a subject having Alzheimer's disease.
251. A pharmaceutical composition according to any one of embodiments 1-58 or 113-140, for use in treating Mild Cognitive Impairment (MCI) associated with Alzheimer's disease in a subject.
252. A pharmaceutical composition according to any one of embodiments 1-58 or 113-140, for use in improving executive function in a subject having Mild Cognitive Impairment (MCI) associated with Alzheimer's disease.
253. A pharmaceutical composition according to any one of embodiments 1-58 or 113-140, for use in improving working memory in a subject having Mild Cognitive Impairment (MCI) associated with Alzheimer's disease.
254. A pharmaceutical composition according to any one of embodiments 1-58 or 113-140, for use in improving learning in a subject having Mild Cognitive Impairment (MCI) associated with Alzheimer's disease.
255. A pharmaceutical composition according to any one of embodiments 1-58 or 113-140, for use in improving learning and working memory in a subject having Mild Cognitive Impairment (MCI) associated with Alzheimer's disease.
256. A pharmaceutical composition according to any one of embodiments 1-58 or 113-140, for use in improving cognition in a subject having Alzheimer's disease.
257. A pharmaceutical composition according to any one of embodiments 1-58 or 113-140, for use in slowing cognitive decline in a subject having Alzheimer's disease.
258. A pharmaceutical composition according to any one of embodiments 1-58 or 113-140, for use in treating mild dementia associated with Alzheimer's Disease.
259. A pharmaceutical composition according to any one of embodiments 1-58 or 113-140, for use in improving executive function in a subject having mild dementia associated with Alzheimer's disease.
260. A pharmaceutical composition according to any one of embodiments 1-58 or 113-140, for use in improving working memory in a subject having mild dementia associated with Alzheimer's disease.
261. A pharmaceutical composition according to any one of embodiments 1-58 or 113-140, for use in improving learning in a subject having mild dementia associated with Alzheimer's disease.
262. A pharmaceutical composition according to any one of embodiments 1-58 or 113-140, for use in improving learning and working memory in a subject having mild dementia associated with Alzheimer's disease.
263. The pharmaceutical composition for use according to any one of embodiments 231-262, wherein the composition is formulated for administering a total daily dose of from about 0.3 mg to about 1.5 mg of Compound I.
264. The pharmaceutical composition for use according to embodiment 263, wherein the composition is formulated for administering a total daily dose of about 1.2 mg of Compound I.
265. The pharmaceutical composition for use according to embodiment 263, wherein the composition is formulated for administering a total daily dose of 1.2 mg of Compound I.
266. The pharmaceutical composition for use according to embodiment 263, wherein the composition is formulated for administering a total daily dose of about 0.9 mg of Compound I.
267. The pharmaceutical composition for use according to embodiment 263, wherein the composition is formulated for administering a total daily dose of 0.9 mg of Compound I.
268. The pharmaceutical composition for use according to embodiment 263, wherein the composition is formulated for administering a total daily dose of about 0.6 mg of Compound I.
269. The pharmaceutical composition for use according to embodiment 263, wherein the composition is formulated for administering a total daily dose of 0.6 mg of Compound I.
270. The pharmaceutical composition for use according to embodiment 263, wherein the composition is formulated for administering a total daily dose of about 0.3 mg of Compound I.
271. The pharmaceutical composition for use according to embodiment 263, wherein the composition is formulated for administering a total daily dose of 0.3 mg of Compound I.
272. The pharmaceutical composition for use according to any one of embodiments 231-271, wherein the treatment comprises administering at least one loading dose and at least one maintenance dose.
273. The pharmaceutical composition for use according to embodiment 272, wherein the treatment comprises administering the loading dose daily for at least 2, 4, 6, 8 or 10 weeks.
274. The pharmaceutical composition for use according to embodiment 272 or 273, wherein the treatment comprises administering the loading dose daily for at least 2 weeks.
275. The pharmaceutical composition for use according to embodiment 272 or 273, wherein the treatment comprises administering the loading dose daily for at least 4 weeks.
276. The pharmaceutical composition for use according to embodiment 272 or 273, wherein the treatment comprises administering the loading dose daily for at least 6 weeks.
277. The pharmaceutical composition for use according to embodiment 272 or 273, wherein the treatment comprises administering the loading dose daily for at least 8 weeks.
278. The pharmaceutical composition for use according to embodiment 272 or 273, wherein the treatment comprises administering the loading dose daily for at least 10 weeks.
279. The pharmaceutical composition for use according to any one of embodiments 272-278, wherein the loading dose is about 1.2 mg daily of Compound I.
280. The pharmaceutical composition for use according to any one of embodiments 272-278, wherein the loading dose is 1.2 mg daily of Compound I.
281. The pharmaceutical composition for use according to any one of embodiments 272-280, wherein the maintenance dose is about 0.9 mg daily of Compound I.
282. The pharmaceutical composition for use according to any one of embodiments 272-281, wherein the maintenance dose is administered daily.
283. The pharmaceutical composition for use according to any one of embodiments 272-282, wherein the maintenance dose is 0.9 mg daily of Compound I.
284. The pharmaceutical composition for use according to any one of embodiments 272-280 or 282, wherein the maintenance dose is about 0.6 mg daily of Compound I.
285. The pharmaceutical composition for use according to any one of embodiments 272-280 or 282, wherein the maintenance dose is 0.6 mg daily of Compound I.
286. Use of a pharmaceutical composition according to any one of embodiments 1-58 or 113-140 for the manufacture of a medicament for treating Huntington's disease in a subject.
287. Use of a pharmaceutical composition according to any one of embodiments 1-58 or 113-140 for the manufacture of a medicament for treating cognitive impairment in a subject having Huntington's disease.
288. Use of a pharmaceutical composition according to any one of embodiments 1-58 or 113-140 for the manufacture of a medicament for treating Mild Cognitive Impairment (MCI) associated with Huntington's disease in a subject.
289. Use of a pharmaceutical composition according to any one of embodiments 1-58 or 113-140 for the manufacture of a medicament for improving executive function in a subject having Mild Cognitive Impairment (MCI) associated with Huntington's disease.
290. Use of a pharmaceutical composition according to any one of embodiments 1-58 or 113-140 for the manufacture of a medicament for improving working memory in a subject having Mild Cognitive Impairment (MCI) associated with Huntington's disease.
291. Use of a pharmaceutical composition according to any one of embodiments 1-58 or 113-140 for the manufacture of a medicament for improving learning in a subject having Mild Cognitive Impairment (MCI) associated with Huntington's disease.
292. Use of a pharmaceutical composition according to any one of embodiments 1-58 or 113-140 for the manufacture of a medicament for improving learning and working memory in a subject having Mild Cognitive Impairment (MCI) associated with Huntington's disease.
293. Use of a pharmaceutical composition according to any one of embodiments 1-58 or 113-140 for the manufacture of a medicament for improving cognition in a subject having Huntington's disease.
294. Use of a pharmaceutical composition according to any one of embodiments 1-58 or 113-140 for the manufacture of a medicament for slowing cognitive decline in a subject having Huntington's disease.
295. Use of a pharmaceutical composition according to any one of embodiments 1-58 or 113-140 for the manufacture of a medicament for treating Parkinson's disease in a subject.
296. Use of a pharmaceutical composition according to any one of embodiments 1-58 or 113-140 for the manufacture of a medicament for treating cognitive impairment in a subject having Parkinson's disease.
297. Use of a pharmaceutical composition according to any one of embodiments 1-58 or 113-140 for the manufacture of a medicament for treating Mild Cognitive Impairment (MCI) associated with Parkinson's disease in a subject.
298. Use of a pharmaceutical composition according to any one of embodiments 1-58 or 113-140 for the manufacture of a medicament for improving executive function in a subject having Mild Cognitive Impairment (MCI) associated with Parkinson's disease.
299. Use of a pharmaceutical composition according to any one of embodiments 1-58 or 113-140 for the manufacture of a medicament for improving working memory in a subject having Mild Cognitive Impairment (MCI) associated with Parkinson's disease.
300. Use of a pharmaceutical composition according to any one of embodiments 1-58 or 113-140 for the manufacture of a medicament for improving learning in a subject having Mild Cognitive Impairment (MCI) associated with Parkinson's disease.
301. Use of a pharmaceutical composition according to any one of embodiments 1-58 or 113-140 for the manufacture of a medicament for improving learning and working memory in a subject having Mild Cognitive Impairment (MCI) associated with Parkinson's disease.
302. Use of a pharmaceutical composition according to any one of embodiments 1-58 or 113-140 for the manufacture of a medicament for improving cognition in a subject having Parkinson's disease.
303. Use of a pharmaceutical composition according to any one of embodiments 1-58 or 113-140 for the manufacture of a medicament for slowing cognitive decline in a subject having Parkinson's disease.
304. Use of a pharmaceutical composition according to any one of claims 1-58 or 113-140 for the manufacture of a medicament for treating Alzheimer's disease in a subject.
305. Use of a pharmaceutical composition according to any one of embodiments 1-58 or 113-140 for the manufacture of a medicament for treating cognitive impairment in a subject having Alzheimer's disease.
306. Use of a pharmaceutical composition according to any one of embodiments 1-58 or 113-140 for the manufacture of a medicament for treating Mild Cognitive Impairment (MCI) associated with Alzheimer's disease in a subject.
307. Use of a pharmaceutical composition according to any one of embodiments 1-58 or 113-140 for the manufacture of a medicament for improving executive function in a subject having Mild Cognitive Impairment (MCI) associated with Alzheimer's disease.
308. Use of a pharmaceutical composition according to any one of embodiments 1-58 or 113-140 for the manufacture of a medicament for improving working memory in a subject having Mild Cognitive Impairment (MCI) associated with Alzheimer's disease.
309. Use of a pharmaceutical composition according to any one of embodiments 1-58 or 113-140 for the manufacture of a medicament for improving learning in a subject having Mild Cognitive Impairment (MCI) associated with Alzheimer's disease.
310. Use of a pharmaceutical composition according to any one of embodiments 1-58 or 113-140 for the manufacture of a medicament for improving learning and working memory in a subject having Mild Cognitive Impairment (MCI) associated with Alzheimer's disease.
311. Use of a pharmaceutical composition according to any one of embodiments 1-58 or 113-140 for the manufacture of a medicament for improving cognition in a subject having Alzheimer's disease.
312. Use of a pharmaceutical composition according to any one of embodiments 1-58 or 113-140 for the manufacture of a medicament for slowing cognitive decline in a subject having Alzheimer's disease.
313. Use of a pharmaceutical composition according to any one of embodiments 1-58 or 113-140 for the manufacture of a medicament for treating mild dementia associated with Alzheimer's Disease.
314. Use of a pharmaceutical composition according to any one of embodiments 1-58 or 113-140 for the manufacture of a medicament for improving executive function in a subject having mild dementia associated with Alzheimer's disease.
315. Use of a pharmaceutical composition according to any one of embodiments 1-58 or 113-140 for the manufacture of a medicament for improving working memory in a subject having mild dementia associated with Alzheimer's disease.
316. Use of a pharmaceutical composition according to any one of embodiments 1-58 or 113-140 for the manufacture of a medicament for improving learning in a subject having mild dementia associated with Alzheimer's disease.
317. Use of a pharmaceutical composition according to any one of embodiments 1-58 or 113-140 for the manufacture of a medicament for improving learning and working memory in a subject having mild dementia associated with Alzheimer's disease.
318. The use according to any one of embodiments 286-317, wherein the total daily dose of Compound I is from about 0.3 mg to about 1.5 mg.
319. The use according to embodiment 318 wherein the total daily dose of Compound I is about 1.2 mg.
320. The use according to embodiment 318, wherein the total daily dose of Compound I is 1.2 mg.
321. The use according to embodiment 318, wherein the total daily dose of Compound I is about 0.9 mg.
322. The use according to embodiment 318, wherein the total daily dose of Compound I is 0.9 mg.
323. The use according to embodiment 318, wherein the total daily dose of Compound I is about 0.6 mg.
324. The use according to embodiment 318, wherein the total daily dose of Compound I is 0.6 mg.
325. The use according to embodiment 318, wherein the total daily dose of Compound I is about 0.3 mg.
326. The use according to embodiment 318, wherein the total daily dose of Compound I is 0.3 mg.
327. The use according to any one of embodiments 286-326, wherein the treatment comprises administering at least one loading dose and at least one maintenance dose.
328. The use according to embodiment 327, wherein the treatment comprises administering the loading dose daily for at least 2, 4, 6, 8 or 10 weeks.
329. The use according to embodiment 327 or 328, wherein the treatment comprises administering the loading dose daily for at least 2 weeks.
330. The use according to 327 or 328, wherein the treatment comprises administering the loading dose daily for at least 4 weeks.
331. The use according to embodiment 327 or 328, wherein the treatment comprises administering the loading dose daily for at least 6 weeks.
332. The use according to embodiment 327 or 328, wherein the treatment comprises administering the loading dose daily for at least 8 weeks.
333. The use according to embodiment 327 or 328, wherein the treatment comprises administering the loading dose daily for at least 10 weeks.
334. The use according to any one of embodiments 327-328, wherein the loading dose is about 1.2 mg daily of Compound I.
335. The use according to any one of embodiments 327-334, wherein the loading dose is 1.2 mg daily of Compound I.
336. The use according to any one of embodiments 320-335, wherein the maintenance dose is administered daily.
337. The use according to any one of embodiments 320-336, wherein the maintenance dose is about 0.9 mg daily of Compound I.
338. The use according to any one of embodiments 320-336, wherein the maintenance dose is 0.9 mg daily of Compound I.
339. The use according to any one of embodiments 320-336, wherein the maintenance dose is about 0.6 mg daily of Compound I.
340. The use according to any one of embodiments 320-336, wherein the maintenance dose is 0.6 mg daily of Compound I.
341. A dosing regimen for administering a therapeutically effective amount of a pharmaceutical composition according to any one of embodiments 1-58 or 113-140 to a subject in need thereof comprising administering to the subject at least one loading dose; and at least one maintenance dose.
342. The dosing regimen according to embodiment 341, wherein the loading dose is administered daily for at least 2, 4, 6, 8 or 10 weeks.
343. The dosing regimen according to embodiment 341 or 342, wherein the loading dose is administered daily for at least 2 weeks.
344. The dosing regimen according to 341 or 342, wherein the loading dose is administered daily for at least 4 weeks.
345. The dosing regimen according to embodiment 341 or 342, wherein the loading dose is administered daily for at least 6 weeks.
346. The dosing regimen according to embodiment 341 or 342, wherein the loading dose is administered daily for at least 8 weeks.
347. The dosing regimen according to embodiment 341 or 342, wherein the loading dose is administered daily for at least 10 weeks.
348. The dosing regimen according to any one of embodiments 341-347, wherein the loading dose is about 1.2 mg daily of Compound I.
349. The dosing regimen according to any one of embodiments 341-347, wherein the loading dose is 1.2 mg daily of Compound I.
350. The dosing regimen according to any one of embodiments 341-347, wherein the maintenance dose is about 0.9 mg daily of Compound I.
351. The dosing regimen according to any one of embodiments 341-347, wherein the maintenance dose is 0.9 mg daily of Compound I.
352. The dosing regimen according to any one of embodiments 341-347, wherein the maintenance dose is about 0.6 mg daily of Compound I.
353. The dosing regimen according to any one of embodiments 341-347, wherein the maintenance dose is 0.6 mg daily of Compound I.
354. A method of treating Huntington's disease in a subject, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%,
355. A method of treating cognitive impairment in a subject having Huntington's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%,
356. A method of treating Mild Cognitive Impairment (MCI) associated with Huntington's disease in a subject, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
357. A method of improving executive function in a subject having Mild Cognitive Impairment (MCI) associated with Huntington's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%,
358. A method of improving working memory in a subject having Mild Cognitive Impairment (MCI) associated with Huntington's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%,
359. A method of improving learning in a subject having Mild Cognitive Impairment (MCI) associated with Huntington's disease, comprising administering to the subject a therapeutically effective amount of a pharmaceutical composition comprising Compound I, or a pharmaceutically acceptable salt thereof, and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
360. A method of improving learning and working memory in a subject having Mild Cognitive Impairment (MCI) associated with Huntington's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
361. A method of improving cognition in a subject having Huntington's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
362. A method for slowing cognitive decline in a subject having Huntington's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
363. A method of treating Parkinson's disease in a subject, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
364. A method of treating cognitive impairment in a subject having Parkinson's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
365. A method of treating Mild Cognitive Impairment (MCI) associated with Parkinson's disease in a subject, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
366. A method of improving executive function in a subject having Mild Cognitive Impairment (MCI) associated with Parkinson's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
367. A method of improving working memory in a subject having Mild Cognitive Impairment (MCI) associated with Parkinson's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
368. A method of improving learning in a subject having Mild Cognitive Impairment (MCI) associated with Parkinson's disease, comprising administering to the subject a therapeutically effective amount of a pharmaceutical composition comprising Compound I, or a pharmaceutically acceptable salt thereof, and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
369. A method of improving learning and working memory in a subject having Mild Cognitive Impairment (MCI) associated with Parkinson's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
370. A method of improving cognition in a subject having Parkinson's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
371. A method for slowing cognitive decline in a subject having Parkinson's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
372. A method of treating Alzheimer's disease in a subject, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
373. A method of treating cognitive impairment in a subject having Alzheimer's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
374. A method of treating Mild Cognitive Impairment (MCI) associated with Alzheimer's disease in a subject, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
375. A method of improving executive function in a subject having Mild Cognitive Impairment (MCI) associated with Alzheimer's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
376. A method of improving working memory in a subject having Mild Cognitive Impairment (MCI) associated with Alzheimer's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
377. A method of improving learning in a subject having Mild Cognitive Impairment (MCI) associated with Alzheimer's disease, comprising administering to the subject a therapeutically effective amount of a pharmaceutical composition comprising Compound I, or a pharmaceutically acceptable salt thereof, and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
378. A method of improving learning and working memory in a subject having Mild Cognitive Impairment (MCI) associated with Alzheimer's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
379. A method of improving cognition in a subject having Alzheimer's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
380. A method for slowing cognitive decline in a subject having Alzheimer's disease, comprising administering to the subject a therapeutically effective amount of a pharmaceutical composition comprising Compound I, or a pharmaceutically acceptable salt thereof, and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
381. A method of treating mild dementia associated with Alzheimer's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
382. A method of improving executive function in a subject having mild dementia associated with Alzheimer's disease, comprising administering to the subject a therapeutically effective amount of a pharmaceutical composition comprising Compound I, or a pharmaceutically acceptable salt thereof, and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
383. A method of improving working memory in a subject having mild dementia associated with Alzheimer's disease, comprising administering to the subject a therapeutically effective amount of a pharmaceutical composition comprising Compound I, or a pharmaceutically acceptable salt thereof, and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
384. A method of improving learning in a subject having mild dementia associated with Alzheimer's disease, comprising administering to the subject a therapeutically effective amount of a pharmaceutical composition comprising Compound I, or a pharmaceutically acceptable salt thereof; and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.
385. A method of improving learning and working memory in a subject having mild dementia associated with Alzheimer's disease, comprising administering to the subject a therapeutically effective amount of a pharmaceutical composition comprising Compound I, or a pharmaceutically acceptable salt thereof, and a pharmaceutically acceptable carrier; wherein following administration of the pharmaceutical composition to the subject, the pharmaceutical composition provides one or more of the following pharmacokinetic parameters:
   a) a Cₘₐₓ of between about 8 and about 25 ng/mL;
   b) an AUCₗₐₛₜ of between about 100 and about 450 ng*h/mL;
   c) an AUC_{inf} of between about 100 and about 500 ng*h/mL;
   d) a tₘₐₓ of between about 2 and about 8 hours; or
   e) a bioavailability of between about 70 and about 100%.

What is claimed is:

1. A pharmaceutical composition comprising
a) Compound I: 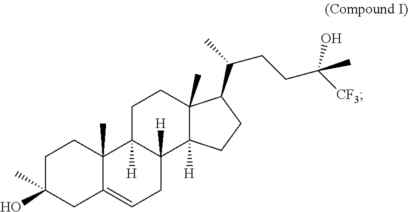
or a pharmaceutically acceptable salt thereof; and
b) one or more glycerides, wherein said glyceride comprises between about 30% and about 100% w/w monoglycerides.

2. The pharmaceutical composition according to claim 1, wherein the pharmaceutical composition comprises Compound I.

3. The pharmaceutical composition according to any one of claims 1-2, wherein the pharmaceutical composition comprises a pharmaceutically acceptable salt of Compound I.

4. The pharmaceutical composition according to any one of claims 1-3, wherein the pharmaceutical composition comprises about 0.2% to about 0.4% w/w Compound I.

5. The pharmaceutical composition according to any one of claims 1-3, wherein the pharmaceutical composition comprises about 3 mg/g of Compound I.

6. The pharmaceutical composition according to any one of claims 1-5, wherein the one or more glycerides is selected from the group consisting of glyceryl mono-/di-caprylocaprate, glyceryl mono-/di-caprylocaprate, glyceryl monocaprylate Type 1, glyceryl monocaprylate Type 1, glyceryl monocaprylocaprate Type 1, glyceryl monolinoleate, and combinations thereof.

7. The pharmaceutical composition according to any one of claims 1-6, wherein the one or more glycerides is selected from the group consisting of glyceryl monocaprylate, glyceryl monolinoleate and a combination thereof.

8. The pharmaceutical composition according to any one of claims 1-7, wherein the pharmaceutical composition comprises glyceryl monocaprylate.

9. The pharmaceutical composition according to any one of claims 1-7, wherein the pharmaceutical composition comprises glyceryl monolinoleate.

10. The pharmaceutical composition according to any one of claims 1-7, wherein the pharmaceutical composition comprises glyceryl monocaprylate and glyceryl monolinoleate.

11. The pharmaceutical composition according to any one of claims 1-10, wherein the amount of the one or more glycerides is from about 20% w/w to about 80% w/w.

12. The pharmaceutical composition according to any one of claims 7, 9 or 10, wherein the amount of glyceryl monolinoleate is from about 20% w/w to about 30% w/w.

13. The pharmaceutical composition according to any one of claims 1-12, further comprising stearoyl polyoxyl-32 glyceride.

14. The pharmaceutical composition according to claim 13, wherein the amount of stearoyl polyoxyl-32 glyceride is from about 5% w/w to about 20% w/w.

15. The pharmaceutical composition according to any one of claims 1-14, wherein the composition further comprises a surfactant.

16. The pharmaceutical composition according to claim 15, wherein the amount of surfactant is from about 10% w/w to about 25% w/w.

17. The pharmaceutical composition according to any one of claim 15-16, wherein the surfactant is vitamin E TPGS.

18. The pharmaceutical composition according to any one of claims 1-17, wherein the composition further comprises an antioxidant.

19. The pharmaceutical composition according to claim 18, wherein the amount of antioxidant is from about 0.005% w/w to about 0.02% w/w.

20. The pharmaceutical composition according to any one of claims 18-19, wherein the antioxidant is butylated hydroxyanisole (BHA).

21. A pharmaceutical composition comprising:
a) about 0.3% w/w Compound I,
b) about 60% w/w glyceryl monocaprylate,
c) about 20% w/w glyceryl monolinoleate,
d) about 20% w/w vitamin E TPGS, and
e) about 0.01% w/w butylated hydroxyanisole.

22. A pharmaceutical composition comprising:
a) about 0.3% w/w Compound I,
b) about 50% w/w glyceryl monocaprylate,
c) about 22.5% w/w glyceryl monolinoleate,
d) about 20% w/w vitamin E TPGS,
e) about 7.5% w/w stearoyl polyoxyl-32 glyceride, and
f) about 0.01% w/w butylated hydroxyanisole.

23. The pharmaceutical composition of any one of claims 1-22, wherein the pharmaceutical composition is an oral dosage form.

24. The pharmaceutical composition of any one of claims 1-23, wherein the pharmaceutical composition is a lipid formulation.

25. The pharmaceutical composition of any one of claims 1-24, wherein the oral dosage form is a soft gelatin capsule, a hardshell capsule or an oral liquid.

26. The pharmaceutical composition according to any one of claims 1-25, wherein following administration of the pharmaceutical composition to a subject, the pharmaceutical composition provides one or more of the following pharmacokinetic parameters:
a) a Cₘₐₓ of between about 8 and about 25 ng/mL;
b) an AUCₗₐₛₜ of between about 100 and about 450 ng*h/mL;
c) an AUC_{inf} of between about 100 and about 500 ng*h/mL;
d) a tₘₐₓ of between about 2 and about 8 hours; or
e) a bioavailability of between about 70 and about 100%.

27. A pharmaceutical composition comprising a therapeutically effective amount of Compound I 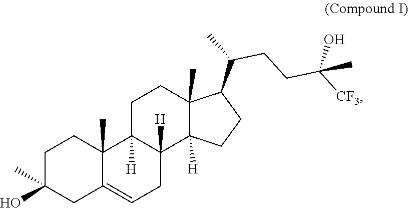
or a pharmaceutically acceptable salt thereof;
and a pharmaceutically acceptable carrier;
wherein following administration of the pharmaceutical composition to a subject, the pharmaceutical composition provides one or more of the following pharmacokinetic parameters:
a) a Cₘₐₓ of between about 8 and about 25 ng/mL;
b) an AUCₗₐₛₜ of between about 100 and about 450 ng*h/mL;
c) an AUC_{inf} of between about 100 and about 500 ng*h/mL;
d) a tₘₐₓ of between about 2 and about 8 hours; or
e) a bioavailability of between about 70 and about 100%.

28. The pharmaceutical composition according to claim 27, wherein the pharmaceutical composition provides a Cₘₐₓ of between about 8 and about 25 ng/mL.

29. The pharmaceutical composition according one of claims 27-28, wherein the pharmaceutical composition provides a tₘₐₓ of between about 2 and about 8 hours.

30. The pharmaceutical composition according to any one of claims 27-29, wherein the pharmaceutical composition provides an AUCₗₐₛₜ of between about 100 and about 450 ng*h/mL.

31. The pharmaceutical composition according to any one of claims 27-30, wherein the pharmaceutical composition provides an AUC_{inf} of between about 100 and about 500 ng*h/mL.

32. The pharmaceutical composition according to any one of claims 27-31, wherein the pharmaceutical composition provides a bioavailability of between about 70% to about 100%.

33. The pharmaceutical composition according to any one of claims 27-32, wherein said pharmacokinetic parameter is obtained following a single administration of the pharmaceutical composition.

34. The pharmaceutical composition according to any one of claims 27-32, wherein said pharmacokinetic parameter is obtained following 2-60 administrations of the pharmaceutical composition.

35. The pharmaceutical composition according to any one of claims 27-34, wherein said pharmacokinetic parameter is obtained under fasted conditions.

36. The pharmaceutical composition according to any one of claims 27-34, wherein said pharmacokinetic parameter is obtained under fed conditions.

37. The pharmaceutical composition according to any one of claims 27-36, wherein the pharmaceutical composition is an oral dosage form.

38. A method of treating a CNS-related condition in a subject, wherein said CNS-related condition is selected from the group consisting of Huntington's disease, Parkinson's disease and Alzheimer's disease comprising administering to the subject a therapeutically effective amount of a pharmaceutical composition according to any one of claims 1-37.

39. A method of treating cognitive impairment having a CNS-related condition in a subject, wherein said CNS-related condition is selected from the group consisting of Huntington's disease, Parkinson's disease and Alzheimer's disease comprising administering to the subject a therapeutically effective amount of a pharmaceutical composition according to any one of claims 1-37.

40. A method of treating Mild Cognitive Impairment (MCI) associated with a CNS-related condition in a subject, wherein said CNS-related condition is selected from the group consisting of Huntington's disease, Parkinson's disease and Alzheimer's, comprising administering to the subject a therapeutically effective amount of a pharmaceutical composition according to any one of claims 1-37.

41. A method of improving executive function in a subject having Mild Cognitive Impairment (MCI) associated with a CNS-related condition, wherein said CNS-related condition is selected from the group consisting of Huntington's disease, Parkinson's disease and Alzheimer's disease comprising administering to the subject a therapeutically effective amount of a pharmaceutical composition according to any one of claims 1-37.

42. A method of improving working memory in a subject having Mild Cognitive Impairment (MCI) associated with a CNS-related condition, wherein said CNS-related condition is selected from the group consisting of Huntington's disease, Parkinson's disease and Alzheimer's disease comprising administering to the subject a therapeutically effective amount of a pharmaceutical composition according to any one of claims 1-37.

43. A method of improving learning in a subject having Mild Cognitive Impairment (MCI) associated with a CNS-related condition, wherein said CNS-related condition is selected from the group consisting of Huntington's disease, Parkinson's disease and Alzheimer's disease comprising administering to the subject a therapeutically effective amount of a pharmaceutical composition according to any one of claims 1-37.

44. A method of improving learning and working memory in a subject having Mild Cognitive Impairment (MCI) associated with a CNS-related condition, wherein said CNS-related condition is selected from the group consisting of Huntington's disease, Parkinson's disease and Alzheimer's disease comprising administering to the subject a therapeutically effective amount of a pharmaceutical composition according to any one of claims 1-37.

45. A method of improving cognition in a subject having a CNS-related condition, wherein said CNS-related condition is selected from the group consisting of Huntington's disease, Parkinson's disease and Alzheimer's disease comprising administering to the subject a therapeutically effective amount of a pharmaceutical composition according to any one of claims 1-37.

46. A method for slowing cognitive decline in a subject having a CNS-related condition, wherein said CNS-related condition is selected from the group consisting of Huntington's disease, Parkinson's disease and Alzheimer's disease comprising administering to the subject a therapeutically effective amount of a pharmaceutical composition according to any one of claims 1-37.

47. The method according to any one of claims 38-46, comprising administering to the subject a total daily dose of from about 0.3 mg to about 1.5 mg of Compound I.

48. The method according to any one of claims 38-46, comprising administering to the subject a total daily dose of about 1.2 mg of Compound I.

49. The method according to any one of claims 38-46, comprising administering to the subject a total daily dose of about 0.9 mg of Compound I.

50. The method according to any one of claims 38-49, comprising administering to the subject at least one loading dose; and at least one maintenance dose.

51. The method according to claim 50, wherein the loading dose is about 1.2 mg daily of Compound I.

52. The method according to any one of claims 50-51, wherein the maintenance dose is about 0.9 mg daily of Compound I.

53. A pharmaceutical composition according to any one of claims 1-37, for use in treating a CNS-related condition in a subject, wherein said CNS-related condition is selected from the group consisting of Huntington's disease, Parkinson's disease and Alzheimer's disease.

54. A pharmaceutical composition according to any one of claims 1-37, for use in treating cognitive impairment in a subject having a CNS-related condition, wherein said CNS-related condition is selected from the group consisting of Huntington's disease, Parkinson's disease and Alzheimer's disease.

55. A pharmaceutical composition according to any one of claims 1-37, for use in treating a subject having Mild Cognitive Impairment (MCI) associated with a CNS-related condition, wherein said CNS-related condition is selected from the group consisting of with Huntington's disease, Parkinson's disease and Alzheimer's disease.

56. A pharmaceutical composition according to any one of claims 1-37, for use in improving executive function in a subject having Mild Cognitive Impairment (MCI) associated with a CNS-related condition, wherein said CNS-related condition is selected from the group consisting of Huntington's disease, Parkinson's disease and Alzheimer's disease.

57. A pharmaceutical composition according to any one of claims 1-37, for use in improving working memory in a subject having Mild Cognitive Impairment (MCI) associated with a CNS-related condition, wherein said CNS-related condition is selected from the group consisting of Huntington's disease, Parkinson's disease and Alzheimer's disease.

58. A pharmaceutical composition according to any one of claims 1-37, for use in improving learning in a subject having Mild Cognitive Impairment (MCI) associated with a CNS-related condition, wherein said CNS-related condition is selected from the group consisting of Huntington's disease, Parkinson's disease and Alzheimer's disease.

59. A pharmaceutical composition according to any one of claims 1-37, for use in improving learning and working memory in a subject having Mild Cognitive Impairment (MCI) associated with a CNS-related condition, wherein said CNS-related condition is selected from the group consisting of Huntington's disease, Parkinson's disease and Alzheimer's disease.

60. A pharmaceutical composition according to any one of claims 1-37, for use in improving cognition in a subject having a CNS-related condition, wherein said CNS-related condition is selected from the group consisting of Huntington's disease, Parkinson's disease and Alzheimer's disease.

61. A pharmaceutical composition according to any one of claims 1-37, for use in slowing cognitive decline in a subject having a CNS-related condition, wherein said CNS-related condition is selected from the group consisting of Huntington's disease, Parkinson's disease and Alzheimer's disease.

62. Use of a pharmaceutical composition according to any one of claims 1-37 for the manufacture of a medicament for treating a CNS-related condition in a subject, wherein said CNS-related condition is selected from the group consisting of Huntington's disease, Parkinson's disease and Alzheimer's disease.

63. Use of a pharmaceutical composition according to any one of claims 1-37 for the manufacture of a medicament for treating cognitive impairment in a subject having a CNS-related condition, wherein said CNS-related condition is selected from the group consisting of Huntington's disease, Parkinson's disease and Alzheimer's disease.

64. Use of a pharmaceutical composition according to any one of claims 1-37 for the manufacture of a medicament for treating Mild Cognitive Impairment (MCI) associated with a CNS-related condition in a subject, wherein said CNS-related condition is selected from the group consisting of Huntington's disease, Parkinson's disease and Alzheimer's disease.

65. Use of a pharmaceutical composition according to any one of claims 1-37 for the manufacture of a medicament for improving executive function in a subject having Mild Cognitive Impairment (MCI) associated a CNS-related condition, wherein said CNS-related condition is selected from the group consisting of with Huntington's disease, Parkinson's disease and Alzheimer's disease.

66. Use of a pharmaceutical composition according to any one of claims 1-37 for the manufacture of a medicament for improving working memory in a subject having Mild Cognitive Impairment (MCI) associated with a CNS-related condition, wherein said CNS-related condition is selected from the group consisting of Huntington's disease, Parkinson's disease and Alzheimer's disease.

67. Use of a pharmaceutical composition according to any one of claims 1-37 for the manufacture of a medicament for improving learning in a subject having Mild Cognitive Impairment (MCI) associated with a CNS-related condition, wherein said CNS-related condition is selected from the group consisting of Huntington's disease, Parkinson's disease and Alzheimer's disease.

68. Use of a pharmaceutical composition according to any one of claims 1-37 for the manufacture of a medicament for improving learning and working memory in a subject having Mild Cognitive Impairment (MCI) associated with a CNS-related condition, wherein said CNS-related condition is selected from the group consisting of Huntington's disease, Parkinson's disease and Alzheimer's disease.

69. Use of a pharmaceutical composition according to any one of claims 1-37 for the manufacture of a medicament for improving cognition in a subject having a CNS-related condition, wherein said CNS-related condition is selected from the group consisting of Huntington's disease, Parkinson's disease and Alzheimer's disease.

70. Use of a pharmaceutical composition according to any one of claims 1-37 for the manufacture of a medicament for slowing cognitive decline in a subject having a CNS-related condition, wherein said CNS-related condition is selected from the group consisting of Huntington's disease, Parkinson's disease and Alzheimer's disease.

71. A dosing regimen for administering a therapeutically effective amount of a pharmaceutical composition according to any one of claims 1-37 to a subject in need thereof comprising administering to the subject at least one loading dose, and at least one maintenance dose.

72. The dosing regimen according to any one of claim 71, wherein the loading dose is about 1.2 mg daily of Compound I.

73. The dosing regimen according to any one of claims 71-72, wherein the maintenance dose is about 0.9 mg daily of Compound I.

74. A method of treating a CNS-related condition in a subject, wherein said CNS-related condition is selected from the group consisting of Huntington's disease, Parkinson's disease and Alzheimer's disease, comprising administering to the subject a therapeutically effective amount of a pharmaceutical composition comprising Compound I, or a pharmaceutically acceptable salt thereof, and a pharmaceutically acceptable carrier; wherein following administration of the pharmaceutical composition to the subject, the pharmaceutical composition provides one or more of the following pharmacokinetic parameters:
a) a Cₘₐₓ of between about 8 and about 25 ng/mL;
b) an AUCₗₐₛₜ of between about 100 and about 450 ng*h/mL;
c) an AUC_{inf} of between about 100 and about 500 ng*h/mL;
d) a tₘₐₓ of between about 2 and about 8 hours; or
e) a bioavailability of between about 70 and about 100%.
